(19) United States
(12) Patent Application Publication
(10) Pub. No.: US 2026/0325625 A1
(43) Pub. Date: Oct. 22, 2026

(54) RECOMBINANT MURINE PNEUMONIA VIRUS EXPRESSING SEVERE ACUTE RESPIRATORY SYNDROME CORONAVIRUS 2 (SARS-COV-2) SPIKE PROTEIN

(71) Applicant: The U.S.A., as represented by the Secretary, Department of Health and Human Services, Bethesda, MD (US)

(72) Inventors: Ursula Buchholz, Silver Spring, MD (US); Shirin Munir, Potomac, MD (US); Cindy Luongo, Bethesda, MD (US); Peter Leon Collins, Kensington, MD (US); Cyril Le Nouen, Bethesda, MD (US); Jaclyn Abbey Kaiser, Ranson, WV (US)

(73) Assignee: The U.S.A., as represented by the Secretary, Department of Health and Human Services, Bethesda, MD (US)

(21) Appl. No.: 19/484,594

(22) PCT Filed: May 17, 2024
(86) PCT No.: PCT/US2024/029917

§ 371 Date: Nov. 14, 2025

Related U.S. Application Data
(60) Provisional application No. 63/502,829, filed on May 17, 2023.

(51) Int. Cl. A61K 39/215; A61K 9/00; A61K 39/00; A61K 45/06; A61P 31/14; C12N 7/00

(52) U.S. Cl. CPC A61K 39/215; A61K 9/0043; A61K 45/06; A61P 31/14; C12N 7/00; A61K 2039/5256; A61K 2039/543; A61K 2039/545; C12N 2760/18621; C12N 2760/18622; C12N 2760/18634; C12N 2760/18643; C12N 2760/18651; C12N 2770/20022; C12N 2770/20034; C12N 2800/22

(57) ABSTRACT

Recombinant murine pneumonia viruses (rMPV) expressing severe acute respiratory syndrome coronavirus 2 (SARS-CoV-2) spike (S) protein and their use as live-attenuated virus vaccines against SARS-CoV-2 are described. The rMPV expressing S protein are safe, immunogenic, and efficacious for protecting a subject against SARS-CoV-2 infection and disease, as demonstrated in a non-human primate model. The rMPV induce a strong systemic and mucosal antibody response against the SARS-CoV-2 S protein. Furthermore, a single dose of the live-attenuated rMPV induced strong protection against SARS-CoV-2 challenge, while two doses were fully protective.

### CROSS REFERENCE TO RELATED APPLICATIONS

This application claims the benefit of U.S. Provisional Application No. 63/502,829, filed May 17, 2023, which is herein incorporated by reference in its entirety.

### FIELD

This disclosure concerns recombinant murine pneumonia virus (rMPV) vectors expressing wild-type or modified severe acute respiratory syndrome coronavirus 2 (SARS-CoV-2) spike (S) protein and use of the vectors, such as for eliciting an immune response against SARS-CoV-2 in a subject.

### INCORPORATION OF ELECTRONIC SEQUENCE LISTING

The electronic sequence listing, submitted herewith as an XML file named 4239-108754-02.xml (773,596 bytes), created on May 16, 2024, is herein incorporated by reference in its entirety.

### BACKGROUND

Since emerging in late 2019, severe acute respiratory syndrome coronavirus 2 (SARS-CoV-2) has caused over 700 million cumulative cases and more than 6 million deaths worldwide. SARS-CoV-2 is primarily transmitted by large droplets and aerosols and is the causative agent of coronavirus infectious disease-19 (COVID-19). Most COVID-19 symptoms are associated with the respiratory tract, but non-respiratory symptoms are documented as well.

Rapid deployment of SARS-CoV-2 vaccines in late 2020 helped reduce the burden of disease associated with the pandemic. As of early 2023, four SARS-CoV-2 vaccines were licensed in the United States: two mRNA-based vaccines, a protein subunit vaccine, and a vaccine based on a replication-incompetent adenovirus vector. All of these are injectable vaccines and are based on the spike (S) surface glycoprotein. These COVID-19 vaccines were effective in reducing the burden of severe COVID-19 and COVID-19 mortality. However, these injectable vaccines do not directly stimulate immunity in the respiratory mucosa, and booster immunizations are required to maintain protection. Furthermore, current COVID-19 vaccines are not highly effective in preventing infection and replication of SARS-CoV-2 at the respiratory mucosal entry sites; transmission rates remain high, allowing for the continued emergence of new variants. Thus, a need exists for the development of improved SARS-CoV-2 vaccines that induce a robust mucosal immune response.

### SUMMARY

Described herein is recombinant murine pneumonia virus (rMPV) expressing severe acute respiratory syndrome coronavirus 2 (SARS-CoV-2) spike (S) protein and its use as a live-attenuated virus vaccine against SARS-CoV-2. The disclosed rMPV are shown herein to be safe, immunogenic, and efficacious for protecting a subject again SARS-CoV-2 infection and disease. The rMPV induce a strong systemic and mucosal antibody response against the SARS-CoV-2 S protein. Furthermore, a single dose of the live-attenuated rMPV induced strong protection against SARS-CoV-2 challenge, while two doses were fully protective.

Provided herein is a live-attenuated rMPV that includes the MPV non-structural protein 1 (NS1), non-structural protein 2 (NS2), nucleoprotein (N), phosphoprotein (P), matrix protein (M), small hydrophobic protein (SH), attachment glycoprotein (G), fusion glycoprotein (F), M2, and polymerase protein (L) genes, and a heterologous gene encoding a SARS-CoV-2 S protein inserted between the MPV NS2 and N genes, or between the MPV N and P genes.

In some aspects, the rMPV includes a genome comprising in a 3′ to 5′ order, a 3′ leader sequence, an MPV NS1 gene, an MPV NS2 gene, a heterologous gene, an MPV N gene, an MPV P gene, an MPV M gene, an MPV SH gene, an MPV G gene, an MPV F gene, an MPV M2 gene, an MPV L gene, and a 5′ trailer sequence, wherein the heterologous gene encodes a SARS-CoV-2 S protein, such as a modified S protein that is stabilized in the pre-fusion conformation and/or has one or more substitutions or deletions to inactivate or mutate a polybasic furin cleavage motif.

In other aspects, the rMPV includes a genome comprising in a 3′ to 5′ order, a 3′ leader sequence, an MPV NS1 gene, an MPV NS2 gene, an MPV N gene, a heterologous gene, an MPV P gene, an MPV M gene, an MPV SH gene, an MPV G gene, an MPV F gene, an MPV M2 gene, an MPV L gene, and a 5′ trailer sequence, wherein the heterologous gene encodes a SARS-CoV-2 S protein, such as a modified S protein that is stabilized in the pre-fusion conformation and/or has one or more substitutions or deletions to inactivate or mutate a polybasic furin cleavage motif.

In some examples of the disclosed rMPV, the S protein includes R682G, R683S, R685S, K986P, and V987P amino acid substitutions (with respect to SEQ ID NO: 33). In other examples, the S protein includes R682G, R683S, R685S, F817P, A892P, A899P, A942P, K986P, and V987P amino acid substitutions (with respect to SEQ ID NO: 33). In other examples, the S protein includes R682G, R683S, R685S, K986P, V987P, Q52H and F456L amino acid substitutions (with respect to SEQ ID NO: 33)

Also provided herein are nucleic acid molecules that include the sequence of the genome of a rMPV disclosed herein, or an antigenomic cDNA or RNA sequence of the genome. Vectors that include such a nucleic acid molecule are further provided. In some aspects, the vector includes a promoter (such as a T7 promoter) operably linked to the nucleic acid molecule. Isolated host cells that include a disclosed nucleic acid molecule or vector are also provided.

Further provided herein is an in vitro method of producing a rMPV. In some aspects, the method includes transfecting MPV-permissive cells with a vector disclosed herein, and plasmids encoding MPV N, P, L and M2-1 proteins; incubating the cell culture for a period of time sufficient to allow for viral replication; and purifying the replicated virus to produce the rMPV. In some examples, the MPV-permissive cells express T7 RNA polymerase, or the MPV-permissive cells are transfected with a plasmid encoding T7 RNA polymerase. Recombinant MPV produced by this method are also provided.

Also provided are immunogenic compositions that include a rMPV disclosed herein and a pharmaceutically acceptable carrier, adjuvant and/or a secondary therapeutic agent. In some aspects, the immunogenic composition is formulated for intranasal administration (such as formulated as a spray, mist or aerosol).

Further provided herein are methods of eliciting an immune response against SARS-CoV-2 in a subject, which include administering to the subject an effective amount of an immunogenic composition disclosed herein. In some aspects, the immunogenic composition is formulated for intranasal administration, and/or can be administered as a single dose or in two (or more) doses. In some examples, the immune response elicited by the immunogenic composition is a protective immune response. In some examples, the immunogenic composition is used in a prime-boost dose regimen as either the prime dose, the boost dose, or both the prime and boost doses.

The foregoing and other features of this disclosure will become more apparent from the following detailed description of several aspects which proceeds with reference to the accompanying figures.

### BRIEF DESCRIPTION OF THE DRAWINGS

FIGS. 1A-1G: Genome organization and characterization of MPV and derivatives in cell culture. (FIG. 1A) Schematic representation of the MPV genome and two versions of the inserted SARS-CoV-2 S ORFs, S and S-2P, inserted between the NS2 and N genes. S is the full-length wild-type SARS-CoV-2 S ORF (aa 1-1,273) of the ancestral SARS-CoV-2 (Wuhan-1), codon-optimized for optimal expression in humans. The S1 and S2 subunits are indicated. The S-2P ORF is a version of the S ORF with two proline substitutions (K986P and V987P), introduced to stabilize S in the prefusion form, and with the furin cleavage site “RRAR” replaced by “GSAS” residues, indicated by arrows. The S or S-2P ORF is flanked by MPV gene start and gene end transcription signals to enable transcription of the S gene as a separate mRNA. Reverse genetics was used to recover the MPV/S. MPV/S-2P, and empty MPV vector. (FIG. 1B) Plaque phenotype of MPV and derivatives in Vero cells. MPV proteins were detected using an MPV-specific rabbit hyperimmune serum, and SARS-CoV-2 S was detected using the human anti-S monoclonal antibody CR3022. Infrared fluorophore-labeled secondary antibodies were used to visualize MPV and S. (FIG. 1C) Multicycle growth kinetics in vitro. Duplicate monolayers of Vero and A549 cells were infected at an MOI of 0.1 with MPV, MPV/S or MPV/S-2P and incubated at 32° C. At the indicated time points, cells were scraped into the supernatants. Supernatants were clarified by centrifugation, and titers were determined by dual-staining immunoplaque assay. Mean titers, standard deviation (not visible due to low deviation) and limit of detection (dotted line; 10 PFU/ml) are shown. (FIG. 1D) Stability of S protein expression by MPV/S and MPV/2-P, measured by dual-staining immunoplaque assay, and expressed as the percentage of S-positive plaques out of total plaques at the indicated time point. (FIG. 1E) Viral proteins in lysates of infected Vero cells. Vero cell monolayers were infected at an MOI of 2 PFU/cell with MPV or derivatives. After incubation at 32° C. for 56 hours, cell lysates were prepared, subjected to SDS-PAGE under denaturing and reducing conditions, and analyzed by Western blot using a rabbit anti-MPV hyperimmune serum that detects MPV G, N and P proteins, and a goat anti-S-2P hyperimmune serum that detects the uncleaved form of S as well as S1 and S2 subunits. A mouse anti-tubulin monoclonal antibody was used to detect tubulin, serving as a loading control. Blots were further incubated with infrared fluorophore-labeled secondary antibodies. Infrared fluorescence was scanned using an Odyssey CLX, and protein bands were visualized and quantified using the Image Studio software, normalized to tubulin, and expressed as relative fluorescence intensity compared to MPV (N, P, G proteins) or MPV/S (S protein). Representative blots with the identity of each protein band indicated (left panel) and data from n=4 different wells from 2 independent experiments (with wells from the same experiment represented by the same symbol; right panel) are shown in the right panel. Individual values and medians are shown (* P<0.05, ** P<0.005, *** P<0.001; two-way ANOVA for N, P, and G proteins with Tukey post-test). (FIG. 1F) Sucrose-purified virions (1 μg/lane) were analyzed by Western blotting (as described above) and silver staining. Molecular weight markers are indicated on the left, and the identities of the protein bands are shown on the right. (FIG. 1G) Electron micrographs with S-specific immunogold labelling of MPV and MPV/S2-P particles to confirm the incorporation of the S protein in MPV/S2-P purified virions. Sucrose-purified MPV and MPV/S-2P virus stocks were incubated with goat anti-S serum and immunogold-labeled secondary antibody. Representative images of filamentous MPV and MPV/S-2P particles are shown. The arrows indicate clusters of gold beads associated with MPV/S-2P particles, indicative of the presence of S, with two areas enlarged for visualization.

FIGS. 2A-2H: Replication and immunogenicity of MPV/S-2P in macaque airways. Three groups of four macaques each were immunized by the IN/IT route (see study design in FIG. 10). Group 1 received a single dose of MPV (empty-vector control), and groups 2 and 3 received MPV/S-2P (6.3 log₁₀ PFU per animal). Results from MPV/S2-P primed animals from groups 2 and 3 were combined for analysis (n=8). Twenty-eight days after the first dose, the four macaques of group 3 received a second dose of MPV/S-2P (boost, 6.3 log₁₀ PFU). (FIG. 2A) Replication of MPV and MPV/S-2P in the upper and lower airways of macaques (left and right panels, respectively) was evaluated by plaque assay from nasopharyngeal swabs (NS) and tracheal lavage (TL) samples, respectively, collected at the indicated days post immunization (pi). The limit of detection of 0.7 log₁₀ PFU/ml and 1 log₁₀ PFU/ml for NS and TL, respectively, is indicated by a dotted line. The numbers per group of macaques with detectable virus replication is indicated. (FIGS. 2B-2H) Immunogenicity of MPV/S2-P in the airways. Immunogenicity in the upper airways (UA) and lower airways (LA) was evaluated from nasal washes (NW) (FIGS. 2B-2D) and bronchoalveolar lavages (BAL) (FIGS. 2E-2H), respectively, collected at the indicated day pi. S-specific and RBD-specific IgG (FIGS. 2B and 2E) and IgA (FIGS. 2C and 2F) antibodies were measured using ELISA and dissociation-enhanced lanthanide fluorescent (DELFIA) assays, respectively. The limit of detection is 1.6 log₁₀ (dotted line). Anti-S dimeric IgA in the UA and LA were also evaluated by DELFIA assay (FIGS. 2D and 2G, respectively). The limit of detection is 1.0 log₁₀ (dotted line). (FIG. 2H) BAL samples also were analyzed for their ability to block the binding of tagged, soluble ACE2 to purified S protein from the vaccine-matched SARS-CoV-2 S protein (Wuhan strain) or to S from the indicated variants of concern (VoCs). ACE2 binding inhibition is expressed as % inhibition relative to a no serum control. Data using S from other VoCs are shown in FIG. 12A. The median, min and max values, 25^{th} and 75^{th} quartile, and individual values are shown. * P<0.05, ** P<0.01, *** P<0.0001, two-way ANOVA with Sidak post-test.

FIGS. 3A-3E: Prime boost regimen of MPV/S-2P induced high level of serum anti-S antibodies with increased avidity, breadth and ADCP activity. (FIGS. 3A-3B) Anti-S- and RBD-specific serum IgG (FIG. 3A) and IgA (FIG. 3B) antibody liters were measured by ELISA or DELFIA assay, respectively. The limit of detection is 3.0 log to (dotted line). IgA and IgG antibody binding avidity to S was evaluated by ELISA with or without treatment with NaSCN, which strips low affinity binding antibodies. Anti-S IgG and IgA serum titers with and without NaSCN treatment were determined and the avidity index (AI) was calculated using the ratio of the NaSCN-treated IgG or IgA titer to the PBS-treated serum IgG or IgA titer. A maximum avidity index of 1 corresponds to no loss of antibody binding to S following NaSCN treatment. (FIG. 3C) Antibody-dependent cellular phagocytosis (ADCP) of serum samples harvested at the indicated time points was determined by incubating S-conjugated GFP beads with macaque serum and then measuring the phagocytic activity of THP-1 monocytes with the S-bound serum antibodies. Data are expressed as the 50% effective concentration (EC₅₀) corresponding to the dilution of serum that resulted in 50% of THP-1 cells being GFP positive for binding and/or phagocytosing the S protein. (FIG. 3D) The 50% SARS-CoV-2 serum neutralizing antibody titers (ND₅₀) against the WA1/2020, B.1.1.7, and B.1.351 isolates was determined on Vero E6 cells. Serum MPV neutralizing antibody titers were determined by 60% plaque reduction neutralization tests (PRNT₆₀) on Vero cells. (FIG. 3E) Inhibition of binding of soluble, tagged ACE2 to the indicated purified S proteins by sera collected at the indicated day pi. Inhibition of ACE2 binding is expressed as % inhibition relative to a no-serum control. Data using S from other VoCs are shown in FIG. 12B. * P<0.05, *** P<0.001, **** P<0.0001, two-way ANOVA with Sidak post-test.

FIGS. 4A-4G: MPV/S-2P immunization induced S-specific B cells in the blood that are restimulated after boost. Peripheral blood mononuclear cells (PBMCs) from immunized macaques were collected on the indicated day pi. PBMCs were stained with a cocktail of fluorochrome-labeled monoclonal antibodies to identify and characterize the B cells and fluorochrome-labelled RBD and S-2P probes to identify S-specific B cells that recognize epitopes of the RBD (RBD⁺/S-2P⁺), or outside the RBD (RBD⁻/S-2P⁺). Analysis was done on live, single, non-naïve (IgD⁻) CD95^{+/-} B cells (see FIG. 13A for gating strategy). (FIG. 4A) Dot plots of B cells from representative MPV- or MPV/S-2P-immunized macaques, with the frequency of RBD⁺ and RBD⁻ S-specific B cells at the indicated time points shown. (FIG. 4B) Frequency of S-specific B cells (RBD⁺ and RBD⁻ combined) in the blood of MPV and MPV/S-2P-immunized macaques at the indicated days pi. The median, min, and max values, 25^{th and} 75^{th} quartile, and individual value are shown. (FIG. 4C) Frequencies of S-specific B cells in the blood of MPV/S-2P immunized macaques that are RBD⁺ or RBD⁻ at the indicated days pi. The median values with ranges are shown (prime n=8; boost n=4 macaques). (FIGS. 4D-4E) Isotype class of RBD⁺ and RBD-S⁻ specific B cells. (FIG. 4D) Representative dot plots of B cells from an MPV/S-2P-immunized macaque identifying the isotype class of the RBD⁺ (top) and RBD⁻ (bottom) S-specific B cells. Frequencies are indicated. (FIG. 4E) Median frequencies with ranges of IgM, IgA, and IgG RBD⁺ (left) and RBD⁻ (right) S-specific B cells at the indicated days pi (prime n=8; boost n=4 macaques). (FIGS. 4F-4G) Expression of CD21 and/or CD27 was measured to characterize the memory phenotype of S-specific B cells as activated memory (AM; CD21/CD27⁺), resident memory (RM; CD21⁺/CD27⁺), CD27⁻ RM (CD21⁺/CD27−) or tissue-resident like memory (TLM; CD21⁻/CD27⁻). (FIG. 4F) CD21 and CD27 expression by IgM, IgA, and IgG RBD⁺ (top) or RBD⁻ (bottom) S-specific B cells from a representative MPV/S-2P-immunized macaque. (FIG. 4G) Median frequencies with ranges of the indicated memory phenotypes of the S-specific B cells at the indicated days pi (prime n=8; boost n=4 macaques).

FIGS. 5A-5J: IN/IT immunization with MPV/S-2P induces S-specific CD4⁺ and CD8⁺ T-cell responses in the blood and lower airways that are restimulated after boost. (FIGS. 5A to 5F) Frequencies of S-specific CD4⁺ and CD8⁺ T cells in blood (FIGS. 5A-5C) or bronchoalveolar lavage (BAL) (FIGS. 5D-5F). PBMCs and BAL cells collected on indicated days post-immunization (pi) or post-challenge (pc) were stimulated with overlapping SARS-CoV-2 S or (BAL only) N peptides or left unstimulated and processed for flow cytometry. Phenotypic analyses were done on live single non-naïve non-regulatory (CD95⁺/Foxp3⁻) CD4⁺ or CD8⁺ T-cells (see FIG. 13B for gating strategy) and indicated frequencies are relative to that population. (FIGS. 5A and 5D) Dot plots showing IFNγ and TNFα expression by CD4⁺ or CD8⁺ T-cells from PBMC (FIG. 5A) or BAL (FIG. 5D) of representative MPV (top) or MPV/S-2P-immunized (bottom) macaques. (FIGS. 5B, 5C, 5E, 5F) Background-corrected frequencies of S-specific IFNγ⁺/TNFα⁺ CD4⁺ (FIGS. 5B, 5E) or CD8⁺ T-cells (FIGS. 5C, 5F) from PBMC (FIGS. 5B, 5C) or BAL (FIGS. 5E, 5F) on indicated days pi or pc. Dotted lines separate the time points after priming with MPV or MPV/S-2P (n=4 and 8 macaques, respectively) and boosting with MPV/S-2P (n=4 macaques) on day 0 and day 28, respectively. (FIGS. 5G to 5J) Expression of proliferation marker Ki-67 by IFNγ⁻/TNFα⁻ S-specific CD4⁺ or CD8⁺ T-cells from blood (FIGS. 5G and 5H) or from BAL (FIGS. 5I and 5J) of MPV/S-2P-immunized macaques. (FIGS. 5G and 5J) Gating and histograms showing evolution of Ki-67 expression by IFNγ⁺/TNFα⁺ CD4⁺ or CD8⁺ S-specific T-cells in blood (FIG. 5G) or lower airways (FIG. 5I) from a representative MPV/S-2P-immunized macaque. The IFNγ⁺/TNFα⁺ T cell population was used for comparison. (FIGS. 5H and 5J) Frequencies of Ki-67⁺ T-cells in IFNγ⁺/TNFα⁺ T-cells from blood (FIG. 5H) or BAL (FIG. 5J). The median, min, and max values, 25^{th} and 75^{th} quartile, and individual value are shown. MPV/S-2P primed and MPV/S2-P primed/boosted macaques are represented by circles or triangles, respectively.

FIGS. 6A-6L: IN/IT immunization with MPV/S-2P induced S-specific Th1 and Th17 CD4⁺ and granzyme B expressing CD⁸⁺ T-cells in lower airways that remained functional after boost and that transition to tissue-resident memory phenotype. Cells obtained by bronchoalveolar lavage (BAL) were stimulated with overlapping S or N peptides or kept unstimulated prior to flow cytometry analysis. (FIGS. 6A to 6D) Phenotype of S-specific IFNγ⁺/TNFα⁺ T-cells in lower airways (LA). (FIGS. 6A and 6B) Representative dot plots showing gating on S-specific IFNγ⁺/TNFα⁺ CD4⁺, CD8⁺, and IFNγ/TNFα⁻ T-cells used for comparison. Histograms show expression of IL-2 (CD4⁺ T-cells only), CD107ab and granzyme B by IFNγ⁺/TNFα⁺ S-specific T-cells on indicated days pi. (FIGS. 6C and 6D) Frequencies of IL-2⁺, CD107ab⁺ and granzyme B⁺ of S-specific IFNγ⁻/TNFα⁺ CD4⁺ (FIG. 6C) or CD8⁺ (FIG. 6D) T-cells from eight MPV/S-2P-primed macaques and four MPV/S-2P-boosted macaques on indicated days. The dotted lines represent the MPV/S-2P boost on day 28 after the first immunization. The median, min, and max values, 25^{th} and 75^{th} quartile, and individual value are shown. MPV/S-2P-primed and -primed/boosted macaques are represented by circles or triangles, respectively. (FIGS. 6E and 6F) MPV/S-2P induced a small population of S-specific Th17 CD4⁺ T-cells that are restimulated after boost. (FIG. 6E) Dot plots from representative macaques showing expression of IL-17 by S-specific non-naïve CD95⁺ CD4⁺ T cells in BAL. Frequencies of IL-17⁺ T-cells are indicated. (FIG. 6F) Background-corrected frequencies of S-specific IL-17⁺ CD4⁺ T-cells from BAL on indicated days pi. Dotted lines separate the time points after priming with MPV or MPV/S-2P (n=4 and 8 macaques, respectively) and after boosting on day 28 with MPV/S-2P (n=4 macaques), respectively. (FIGS. 6G to 6L) S-specific IFNγ⁺/TNFα⁺ CD4⁺ (FIGS. 6G and 6H), CD8⁺ (FIGS. 6I and 6J) and S-specific IL-17⁺ CD4⁺ T cells (FIGS. 6K and 6L) transition to memory phenotype. (FIGS. 6G, 6I and 6K) Representative dot plots showing gating on S-specific IFNγ⁺/TNFα⁺ CD4⁺ (FIG. 6G), CD8⁺ (FIG. 6I) and S-specific IL-17⁺ CD4⁺ T cells (FIG. 6K) (left panels). CD69 and CD103 were used to differentiate circulating (CD69⁻/CD103⁻) and tissue-resident memory [Trm; CD69⁺/CD103⁻, CD69⁺/CD103⁺, CD69⁻/CD103⁺] S-specific T cells from LA (right panels, % indicated). (FIGS. 6H, 6J and 6L) The median % with range of circulating and each of the three Trm S-specific IFNγ⁻/TNFα⁻ CD4⁺ (FIG. 6H), CD8⁺ (FIG. 6J) or S-specific IL-17⁺ CD4⁺ T cell subsets present on indicated days in BAL of eight MPV/S-2P-primed macaques and four MPV/S-2P-boosted macaques are stacked. The medians and ranges are shown.

FIGS. 7A-7C: No detectable SARS-CoV-2 challenge virus replication in macaques that received a prime/boost regimen of MPV/S-2P. On days 31 or 32 post-prime (MPV- and MPV/S-2P-immunized macaques; two animals per group per day for technical reasons) or on day 58 (corresponding to day 30 post-boost (MPV/S-2P-immunized macaques, n=4), macaques were challenged by IN/IT with 6.3 log₁₀ TCID₅₀ total of the vaccine-match SARS-CoV-2 WA1/2020 strain (FIG. 10). (FIGS. 7A-7B) SARS-CoV-2 replication in the upper (FIG. 7A) and lower airways (FIG. 7B) following challenge. Nasopharyngeal swabs (NS) and bronchoalveolar lavages (BAL) were collected at the indicated days post-challenge (pe) and SARS-CoV-2 subgenomic E (sgE) mRNA and genomic N (gN) RNA was quantified by RT-qPCR and expressed in log₁₀ copy number per ml of fluid. (FIG. 7C) SARS-CoV-2 quantification from lung tissues. On day 6 pc, animals were euthanized and six regions of lung tissues from each macaque were harvested for quantification of SARS-CoV-2 sgE and gN by RT-qPCR and expressed in log₁₀ copy number per g of tissue. In each graph, the number of macaques with detectable sgE and/or gN is indicated. Limit of detection is 2.85 log₁₀ copies/ml for NS and BAL fluid and 3.6 log₁₀ copies/g for lung tissue. * P<0.05, ** P<0.01, two-way ANOVA with Sidak post-test.

FIGS. 8A-8I: Replication and immunogenicity of MPV/S and MPV/S-2P in rhesus macaques (experiment #1). (FIG. 8A) Timeline of the macaque study to evaluate safety, replication and immunogenicity of MPV/S and MPV/S-2P. Three groups of four macaques each were immunized by the IN/IT route with 6.3 log₁₀ PFU of MPV, MPV/S, and MPV/S-2P. Macaques were monitored daily and nasopharyngeal swab (NS), tracheal lavage (TL), nasal wash (NW) and serum were collected at the indicated days post-immunization (pi). (FIG. 8B) Replication of MPV and derivatives in the upper and lower airways of macaques. Vaccine titers were evaluated by plaque assay on Vero cells from NS and TL collected at the indicated day pi and expressed in log₁₀ PFU/ml. The limits of detection (0.7 log₁₀ for NS; 1 log₁₀ for TL) are indicated by a dotted line. The medians, min, and max values, 25^{th} and 75^{th} quartiles, and individual values are shown. The P value for the significant difference in TL results on day 6 is indicated (two-way ANOVA with Tukey post-test; P<0.05). The number of macaques in each group with replicating virus is indicated in each graph. (FIGS. 8C-8F) Anti-S antibody response in the upper airways (FIGS. 8C-8D) and serum (FIGS. 8E-8F) of immunized macaques. The levels of anti-S and anti-receptor binding domain (RBD) IgG (FIGS. 8C, 8E) and IgA (FIGS. 8D, 8F) in immunized macaques was evaluated by ELISA (FIGS. 8C, 8E) or dissociation-enhanced lanthanide fluorescence immunoassay (DELFIA) (FIGS. 8D, 8F), respectively, from NW samples (FIGS. 8C, 8D) or serum (FIGS. 8E, 8F) collected at the indicated days pi. The level of anti-S and anti-RBD IgG antibodies in the plasma of 23 SARS-CoV-2 convalescent individuals was also determined for comparison. The limits of detection are 1.6 and 3 log₁₀ for mucosal and serum antibody titers, respectively. (FIG. 8G) Neutralizing activities of serum samples. The serum 50% neutralization titer (ND₅₀) against the vaccine-match SARS-CoV-2 WA1/2020 strain and the 60% plaque reduction neutralization titer (PRNT₆₀) against the vaccine vector MPV were determined. The lower limit of detection is 0.8 log₁₀ ND₅₀ or PRNT₆₀ while the upper limit of detection for MPV neutralizing antibodies is 4.01 log₁₀ PRNT₆₀. The median, min, and max values, 25^{th} and 75^{th} quartile, and individual value are shown. * P<0.05, ** P<0.01, two-way ANOVA with Sidak post-test. (FIG. 8H) Inhibition of binding of soluble, tagged ACE2 to the indicated purified S proteins by sera collected at the indicated day pi. Inhibition of ACE2 binding is expressed as % inhibition relative to a no-serum control. Binding inhibition to S proteins from other VoCs is shown in FIG. 12B. (A-D) The medians, min and max values, 25^{th} and 75^{th} quartile, and individual value are shown. * P<0.05, ** P<0.01, *** P<0.005, **** P<0.0001; two-way ANOVA with Tukey post-test. (FIG. 8I) Inhibition of ACE2 binding to purified SARS-CoV-2 spike protein by sera from MPV, MPV/S, and MPV/S-2P immunized macaques. Sera from MPV, MPV/S, or MPV/S-2P immunized macaques were collected on the indicated day pi. The relative inhibition of binding of soluble, tagged ACE2 to purified SARS-CoV-2 S proteins from the indicated variants of concern was evaluated and expressed as % ACE2 binding inhibition relative a no-serum control. The median, min, and max values, 25^{th} and 75^{th} quartile, and individual value are shown. *P<0.05, ** P<0.01, *** P<0.001, **** P<0.0001, two-way ANOVA with Tukey post-test.

FIG. 9: Vital signs of macaques immunized with MPV, MPV/S and MPV/S-2P (experiment #1). The timeline of sampling for study #1 is described in FIG. 8. Three groups of four macaques each were immunized IN/IT with 6.3 log₁₀ PFU of MPV, MPV/S or MPV/S-2P. Weight, respiration rate, oxygen saturation, heart rate, and rectal temperature were monitored on the indicated days post immunization (pi). MPV, MPV/S, and MPV/S-2P-immunized macaques are indicated, with each macaque represented by a different symbol.

FIG. 10: Design of macaque study #2, which investigated the replication, immunogenicity, and protective efficacy of an MPV/S-2P prime and an MPV/S2-P prime/boost immunization regimen. Timeline of sampling of macaque study #2 in which three groups of four macaques each were immunized by the IN/IT route. Group 1 received a single dose of MPV (empty-vector control), and groups 2 and 3 received MPV/S-2P (6.3 log₁₀ PFU per animal). Twenty-eight days after the first dose, macaques of group 3 received a second dose of MPV/S-2P (boost, 6.3 log₁₀ PFU). On days 30-32 after immunization with a single dose (groups 1 and 2) or the second of two doses (group 3), macaques were challenged by the IN/IT route with 6.3 log₁₀ TCID₅₀ of the vaccine-matched WA1/2020 SARS-CoV-2 strain. All macaques were euthanized on day 6 post-challenge.

FIGS. 11A-11C: Vitals signs of macaques in study #2. Three groups of four macaques each were immunized with MPV or with one or two doses of MPV/S-2P and challenged as described in FIG. 10. Weight, respiration rate, oxygen saturation, heart rate, and rectal temperature were monitored on the indicated days post-immunization (pi) or post-challenge (pc). Macaques immunized with MPV, MPV/S-2P (one dose only) and MPV/S-2P (two doses) are indicated, with each macaque represented by a symbol.

FIGS. 12A-12B: Inhibition of ACE2 binding inhibition to purified S proteins by mucosal and serum samples from MPV/S-2P immunized macaques. Three groups of four macaques each were immunized with MPV or one or two doses of MPV/S-2P as described in FIG. 10. Bronchoalveolar lavage (BAL) (FIG. 12A) and serum samples (FIG. 12B) from MPV and MPV/S-2P immunized macaques were harvested on the indicated days or weeks pi. The relative ACE2 binding inhibition to SARS-CoV-2 S protein from the indicated variants of concern by the BAL (FIG. 12A) or serum (FIG. 12B) samples was evaluated and expressed as % ACE2 binding inhibition relative a no-serum control. The median, min, and max values, 25^{th} and 75^{th} quartile, and individual value are shown. *P<0.05, ** P<0.01, *** P<0.001, **** P<0.0001, two-way ANOVA with Sidak post-test.

FIGS. 13A-13B: Gating strategy used to identify spike-specific B cells and spike-specific CD4⁺ and CD8⁺ T-cells from the blood or bronchoalveolar lavage of immunized macaques. (FIG. 13A) Representative flow cytometry dot plots of cells isolated from a PBMC sample that describe the typical gating strategy used to identify the S-specific B cell populations described in FIG. 4. Live cells were gated using the live/dead staining and B cell marker CD20, and lymphocytes were further identified based on forward and side scatter areas. Next, singlets were identified with a first gate based on forward scatter height and forward scatter area followed by a second gate based on side scatter height and side scatter area. An additional live/dead gate was used to exclude any remaining dead cells as well as CD3⁺, CD14⁺, and CD16⁺ cells. Live single B cells identified using CD19 and CD20 and naïve (IgD⁺) B cells were removed from the analysis. Thus S-specific B cells were identified in the live, single, CD3⁻, CD14⁻ CD16⁻ CD19⁺ CD20⁺ IgD⁻ CD95^{+/-} B cell population. Then, tagged RBD and S-2P protein probes were used to identify RBD and/or S-2P-binding B cells, which were used for additional phenotypic analysis described in FIG. 4. (FIG. 13B) Representative flow cytometry dot plots of cells isolated from a BAL sample, describing the typical gating strategy used to identify the CD4⁺ and CD8⁺ T cell populations described in FIG. 5 and FIG. 6. The same gating strategy was applied to identify and analyze the CD4⁺ and CD8⁺ T-cells from PBMC isolated from the blood and after SARS-CoV-2 challenge (FIG. 14 and FIG. 16, respectively). Live cells were first gated based on a live/dead staining and forward scatter area. Next, lymphocytes were identified based on forward and side scatter areas. Then, singlets were selected using a first gate based on forward scatter height and forward scatter area followed by a second gate based on side scatter height and side scatter area. An additional live/dead gating was performed to discard any remaining dead cells. The live single CD3⁺ IFNγ⁺ T-cells were next gated using CD3 and IFNγ. As CD3 expression can be downregulated on activated T-cells, a wide CD3 gate was applied. IFNγ⁺ CD4+ or IFNγ⁺ CD8⁺ T cells were next identified using a CD4 or CD8 antibody. An additional CD8 or CD4 gating was performed to remove potentially remaining CD8⁺ or CD4⁺ T-cells from the CD4⁺ or CD8⁺ T-cell populations, respectively. Finally, non-naïve, non-regulatory CD4⁺ or CD8⁺ T-cells were identified using CD95 and Foxp3, respectively. The phenotypic analysis described in FIGS. 5, 6, 14, and 16 were performed on live single CD3⁺ CD4⁺ CD8⁻ CD95⁺ Foxp3⁻ or live single CD3⁺ CD8⁺ CD4⁻ CD95⁺ Foxp3⁻ T-cells.

FIGS. 14A-14H: Phenotype of SARS-CoV-2 S-specific CD4⁺ and CD8⁺ T-cells in the blood of the MPV/S-2P immunized macaques. Three groups of four macaques each were immunized with MPV or one or two doses of MPV/S-2P as described in FIG. 10. (FIGS. 14A, 14B) Dot blots of the CD4⁺ (FIG. 14A) or CD8⁺ (FIG. 14B) T-cells from the blood of representative MPV/S-2P-immunized macaques describing the gating of S-specific IFNγ⁺/TNFα⁺ CD4⁺ (FIG. 14A) or CD8⁺ (FIG. 14B) T-cells. The levels of expression of IL-2 (for CD4⁺ T-cells only), CD107ab and granzyme B by the IFNγ⁻/TNFα⁺ CD4⁺ or CD8⁺ T-cells from representative macaques are shown as histograms on the indicated days pi with the IFNγ⁻/TNFα⁻ CD4⁺ or CD8⁺ T-cells used for comparison. (FIGS. 14C-14D) % of IFNγ⁺/TNFα⁺ CD4⁺ (FIG. 14C) or CD8⁺ (FIG. 14D) T-cells in the blood of the MPV/S-2P-immunized macaques that expressed TL-2 (for CD4⁺ T-cells only), CD107ab or granzyme B on the indicated days pi. Dotted lines indicate the boost with MPV/S-2P, 28 days after the first immunization. MPV/S-2P primed and MPV/S2-P primed/boosted macaques are represented by circles or triangles, respectively. The median, min, and max values, 25^{th} and 75^{th} quartile, and individual value are shown. (FIGS. 14E-14H) Lack of expression of tissue-resident memory (Trm) markers by S-specific CD4⁺ and CD8⁺ T cells from blood. (FIGS. 14E and 14G) Representative dot plots showing the gating on S-specific IFNγ⁺/TNFα⁺ CD4⁺ (FIG. 14E) or CD8⁺ (FIG. 14G) T-cells (left panels). CD69 and CD103 were used to differentiate circulating (CD69⁻ CD103⁻) and tissue-resident memory [Trm; CD69⁺ CD103⁻, CD69⁺ CD103⁺ and CD69⁻ CD103⁺] S-specific IFNγ⁺/TNFα⁺ T-cells isolated from blood (right panels, % indicated). (FIGS. 14F and 14H) The median % of the circulating and each of the three Trm S-specific IFNγ⁺/TNFα⁺ CD4⁺ (FIG. 14F) or CD8⁺ (FIG. 14H) T-cell subsets present in blood of MPV/S-2P-immunized macaques on indicated days are stacked (median and range are shown). N=eight MPV/S-2P-primed macaques and n=four MPV/S-2P-boosted macaques.

FIGS. 15A-15C: Cytokine concentration in BAL of MPV/S-2P immunized macaques. Three groups of four macaques each were immunized with MPV or MPV/S-2P as described in FIG. 10. Four MPV-immunized macaques and six of eight MPV/S-2P immunized macaques with the strongest T cell responses in the BAL were chosen for analysis of the post-immunization cytokine response in the lower airways. BAL fluid collected on the indicated days after receipt of MPV or a single dose of MPV/S-2P was concentrated tenfold, and the concentration of 36 different cytokines was evaluated by a multiplex bead-based immunoassay. Th1 related cytokines (FIG. 15A), Th2 related cytokines (FIG. 15B), and other cytokines (FIG. 15C) are shown. The median, min, and max values, 25^{th} and 75^{th} quartile, and individual value are shown. *P<0.05, **P<0.01, two-way ANOVA with Sidak post-test.

FIGS. 16A-16D: Phenotype of S-specific IFNγ⁺/TNFα⁺ CD4⁺ and CD8⁺ T cells in the blood and lower airways of immunized macaques before and after SARS-CoV-2 challenge. Three groups of four macaques each were immunized with MPV or MPV/S-2P as described in FIG. 10. (FIGS. 16A and 16B) Background-corrected frequencies of S-specific IFNγ⁺/TNFα⁺ CD4⁺ or CD8⁺ T-cells from blood (FIG. 16A) or airways (FIG. 16B) on day 28 pi and on day 6 post challenge (performed on days 31-32 pi (groups 1 and 2) or 58 pi (group 3)). These frequencies are similar to those shown in FIGS. 5B-5C, and FIGS. 5E-5F for the blood and airways, respectively. (FIGS. 16C and 16D) % and median fluorescence intensity (MFI) of proliferation marker Ki-67 by IFNγ⁻/TNFα⁺ CD4⁺ (top panels) or CD8⁺ (bottom panels) T-cells from blood (FIG. 16C) or airways (FIG. 16D).

FIGS. 17A-17C: Genome maps of recombinant MPV (rMPV) expressing different versions of the SARS-CoV-2 S protein. (FIG. 17A, middle) Schematic of the MPV genome. (FIG. 17A, top) Five constructs in which different versions of the SARS-CoV-2 S ORF were inserted between the MPV NS2 and N genes. Shown are: (i) wild-type (WT) S ORF encoding amino acids 1-1,273 of the ancestral Wuhan-1 strain of SARS-CoV-2 (GENBANK accession number MN908947); (ii) Codon-optimized WT S ORF (referred to herein as “MPV/S”); (iii) S-2P ORF, encoding an S protein with K986P/V987P substitutions to stabilize the S protein in the prefusion conformation (see Wrapp et al., Science 367:1260-1263, 2020); (iv) codon-optimized S-2P ORF (referred to herein as “MPV/S-2P”); and (v) codon-optimized S-TMCT ORF, encoding an S protein in which the transmembrane/cytoplasmic tail (TMCT) of the S protein (residues 1213-1273) is replaced with a sequence encoding the 46 C-terminal amino acids of the MPV fusion (F) protein. (FIG. 17A, bottom) Five constructs in which different versions of the SARS-CoV-2 S ORF were inserted between the MPV N and P genes. Shown are: (vi) WT S ORF; (vii) codon-optimized WT S ORF; (viii) S2-P ORF; (ix) codon-optimized S-2P ORF; and (x) codon-optimized S-TMCT ORF. (FIG. 17B) Four additional constructs in which different versions of the SARS-CoV-2 S ORF were inserted between the MPV NS2 and N genes. Shown are: (xi) codon-optimized S-6P ORF, encoding a pre-fusion stabilized S protein containing six proline substitutions (see Hsieh et al., Science 369:1501-1505, 2020); (xii) codon-optimized Delta variant (B.1.617.2) S-6P ORF; (xiii) codon-optimized Omicron variant (B.1.1.529) S-6P ORF; and (xiv) codon-optimized Omicron variant (BA.5) S2-P ORF. (FIG. 17C) Two constructs in which different versions of the XBB.1.5 SARS-CoV-2 variant S ORF were inserted between the MPV NS2 and N genes. Shown are: (xv) codon-optimized XBB.1.5 variant S2-P ORF; and (xvi) codon-optimized XBB.1.5 variant with Q52H and F456L substitutions and S2-P substitutions. In FIGS. 17A-17C, dark grey indicates ORFs that were codon-optimized for maximal expression in human cells. The approximate locations of the S1 and S2 subunits are indicated. For all constructs, the S ORF was modified to be flanked by putative MPV gene start and gene end transcription signals to enable transcription of the S gene as a separate mRNA. MPV full-length cDNAs were used to generate the corresponding vaccine candidates by reverse genetics, along with the empty-vector control MPV.

FIG. 18: Linear correlation of MPV/S-2P plaque forming units and copy numbers. Clinical study material of MPV/S-2P was manufactured according to current good manufacturing practice (cGMP) on WHO Vero cells. The final vialed material had a titer (potency) of 6.1 log₁₀ PFU per mL. Tenfold serial dilutions of the study material were prepared in Microtest M4RT viral transport medium (Thermo Fisher). Titers of each dilution were confirmed by immunoplaque assay on Vero cells, and copy numbers for each dilution were determined by TAQMAN assay using a primer/probe set specific to the MPV F ORF. Titers determined by MPV immunoplaque assay (log₁₀ PFU per mL) are displayed on the x-axis, and copy numbers (log₁₀ per mL) determined by TAQMAN assay are displayed on the y-axis. Serial dilutions of a DNA plasmid were used as a standard to determine copy numbers. This figure shows the linear correlation of immunoplaque titers (log₁₀ PFU/mL) and copy numbers (log₁₀/mL) (R2=0.9820). The limits of detection of each assay are shown by a dotted line. The results revealed a linear correlation between these two assays.

### SEQUENCE LISTING

The nucleic acid and amino acid sequences listed in the accompanying sequence listing are shown using standard letter abbreviations for nucleotide bases, and single letter code for amino acids, as defined in 37 C.F.R. 1.822. Only one strand of each nucleic acid sequence is shown, but the complementary strand is understood as included by any reference to the displayed strand. In the accompanying sequence listing:

SEQ ID NO: 1 is the nucleotide sequence of a full-length clone of wild-type rMPV.

SEQ ID NO: 2 is the nucleotide sequence of a plasmid of the full-length clone of wild-type MPV (pMPV).

SEQ ID NO: 3 is the nucleotide sequence of a rMPV with L_{Max} modification.

SEQ ID NO: 4 is the nucleotide sequence of a plasmid of rMPV with L_{Max} modification.

SEQ ID NO: 5 is the nucleotide sequence of rMPV/NS2N/S (WT).

SEQ ID NO: 6 is the nucleotide sequence of pMPV/NS2N/S (WT).

SEQ ID NO: 7 is the nucleotide sequence of rMPV/NS2N/S (GSopt).

SEQ ID NO: 8 is the nucleotide sequence of pMPV/NS2N/S (GSopt).

SEQ ID NO: 9 is the nucleotide sequence of rMPV/NS2N/S-2P.

SEQ ID NO: 10 is the nucleotide sequence of pMPV/NS2N/S-2P.

SEQ ID NO: 11 is the nucleotide sequence of rMPV/NS2N/S-2P (GSopt).

SEQ ID NO: 12 is the nucleotide sequence of pMPV/NS2N/S-2P (GSopt).

SEQ ID NO: 13 is the nucleotide sequence of rMPV/NS2N/S-TMCT (GSopt).

SEQ ID NO: 14 is the nucleotide sequence of pMPV/NS2N/S-TMCT (GSopt).

SEQ ID NO: 15 is the nucleotide sequence of rMPV/NP/S (WT).

SEQ ID NO: 16 is the nucleotide sequence of pMPV/NP/S (WT).

SEQ ID NO: 17 is the nucleotide sequence of rMPV/NP/S (GSopt).

SEQ ID NO: 18 is the nucleotide sequence of pMPV/NP/S (GSopt).

SEQ ID NO: 19 is the nucleotide sequence of rMPV/NP/S-2P.

SEQ ID NO: 20 is the nucleotide sequence of pMPV/NP/S-2P.

SEQ ID NO: 21 is the nucleotide sequence of rMPV/NP/S-2P (GSopt).

SEQ ID NO: 22 is the nucleotide sequence of pMPV/NP/S-2P (GSopt).

SEQ ID NO: 23 is the nucleotide sequence of rMPV/NP/S-TMCT (GSopt).

SEQ ID NO: 24 is the nucleotide sequence of pMPV/NP/S-TMCT (GSopt).

SEQ ID NO: 25 is the nucleotide sequence of rMPV/S-6P/B.1.617.2 Delta (GSopt).

SEQ ID NO: 26 is the nucleotide sequence of pMPV/S-6P/B.1.617.2 Delta (GSopt).

SEQ ID NO: 27 is the nucleotide sequence of rMPV/S-6P/B.1.1.529 Omicron (GSopt).

SEQ ID NO: 28 is the nucleotide sequence of pMPV/S-6P/B.1.1.529 Omicron (GSopt).

SEQ ID NO: 29 is the nucleotide sequence of rMPV/S-2P/BA.5 Omicron (GSopt).

SEQ ID NO: 30 is the nucleotide sequence of pMPV/S-2P/BA.5 Omicron (GSopt).

SEQ ID NO: 31 is the nucleotide sequence of rMPV/S-6P (GSopt).

SEQ ID NO: 32 is the nucleotide sequence of pMPV/S-6P (GSopt).

SEQ ID NO: 33 is the amino acid sequence of the S protein from the Wuhan-1 strain of SARS-CoV-2.

SEQ ID NO: 34 is the amino acid sequence of the S-2P protein.

SEQ ID NO: 35 is the amino acid sequence of the S-TMCT (GSopt) protein.

SEQ ID NO: 36 is the amino acid sequence of the S-6P/B.1.617.2 Delta (GSopt) protein.

SEQ ID NO: 37 is the amino acid sequence of the S-6P/B.1.1.529 Omicron (GSopt) protein.

SEQ ID NO; 38 is the amino acid sequence of the S-2P/BA.5 Omicron (GSopt) protein.

SEQ ID NO: 39 is the amino acid sequence of the S-6P (GSopt) protein.

SEQ ID NOs: 40-45 are oligonucleotide primer and probe sequences.

SEQ ID NOs: 46-49 are nucleic acid fragments and modified fragments of the S ORF.

SEQ ID NO: 50 is the nucleotide sequence of rMPV/S-2P/XBB.1.5 (GSopt).

SEQ ID NO: 51 is the nucleotide sequence of pMPV/S-2P-XBB.1.5 (GSopt).

SEQ ID NO: 52 is the nucleotide sequence of rMPV/S-2P/XBB.1.5/F456L (GSopt).

SEQ ID NO: 53 is the nucleotide sequence of pMPV/S-2P/XBB.1.5/F456L (GSopt).

SEQ ID NO: 54 is the amino acid sequence of the S-2P/XBB.1.5 protein.

SEQ ID NO: 55 is the amino acid sequence of the S-2P/XBB.1.5/F456L protein.

### DETAILED DESCRIPTION

### 1. Introduction

Murine pneumonia virus (MPV) has been previously evaluated as a live-attenuated viral vector (Brock et al., J Virol 92(17):e00723-18, 2018). MPV, an orthopneumovirus, is the murine homolog of respiratory syncytial virus (RSV). MPV is a strong live vector vaccine platform for vaccines against respiratory viruses because it is highly attenuated in non-human primates, presumably due to a strong host range restriction; it has a tropism for the respiratory tract; and the pre-existing MPV anti-vector immunity in humans is low (Brock et al., J Virol 86:5829-5843, 2012). Although MPV replication in nonhuman primates was highly restricted, MPV vectors expressing RSV F still induced strong serum RSV-neutralizing antibody responses that were comparable to those induced by wild-type RSV (Brock et al., J Virol 92(17):e00723-18, 2018; Brock et al., J Virol 86:5829-5843, 2012).

Despite the successful generation of rMPV expressing the RSV F protein, development of a vectored vaccine based on pneumoviruses (or the related paramyxoviruses) is complicated and unpredictable. There are few known examples of vectored vaccine candidates based on pneumoviruses (the family containing MPV), with more examples available for the related paramyxovirus family (e.g., parainfluenza viruses). The genomes of pneumoviruses and paramyxoviruses range from approximately 13,000 to more than 19,000 nucleotides (14,886 nucleotides for MPV strain 15), and thus plasmids bearing cDNAs of the viral genome or antigenome (a positive-sense copy of the genome) are correspondingly large and difficult to propagate and manipulate. Issues of DNA instability during propagation in bacteria have been a significant problem in some cases, such as with the genome of RSV, which necessitated strategies for identifying and deleting an unstable region of the genome (Bukreyev er al, J Virol 75:12128-12140, 2001) or using an artificial bacterial chromosome as vector (Hotard et al., Virology 434:129-136, 2012). In some instances, particular inserts or insertion sites are problematic, yielding phenotypically-unstable viruses, viruses refractory to recovery, over-attenuated viruses, or mutated viruses, including viruses in which the foreign insert has been silenced.

In some cases, the use of particular genome insertion sites resulted in substantial restriction of vector replication in vitro and/or in vivo for unknown reasons. In one such case, the use of gene position 3 for insertion of a heterologous ORF to express the RSV F protein from a chimeric bovine/human parainfluenza virus 3 (B/HPIV3) was particularly restrictive (Liang et al., J Virol 2014 88:4237-4250). In another instance, the use of gene position 2 to express the RSV F protein from one version of attenuated human parainfluenza virus 1 (HPIV1) was unexpectedly very difficult to recover and resulted in various adventitious mutations to the vector and/or the insert, while in another version of attenuated HPIV1, this problem was not observed (Mackow et al., J Virol 89:10319-10332, 2015).

The use of HPIV2 as a vector has generally resulted in highly attenuated viruses for unknown reasons. With some vectors, a steep gradient of expression has been observed for different insertion sites in the vector (Liang et al., J Virol 2014 88:4237-50), while in other cases this was not observed (Mackow et al., J Virol 89:10319-32, 2015). Furthermore, plaque size instability is sometimes observed, such as with a recombinant B/HPIV3 vector encoding RSV F protein (Liu et al., PLoS One 15(2):e0228572, 2020). In another study, the translational initiation site of the RSV F insert in a B/HPIV3 vector was designed to be preceded by a so-called “Kozak” sequence of nine nucleotides that is widely thought to provide optimal efficiency of initiation and expression (Liu et al., PLoS One 15(2):e0228572, 2020), but paradoxically this was found to reduce rather than enhance expression. Loss of expression of a heterologous antigen is also a challenge. For example, when a B/HPIV3 vector expressing the RSV F protein was evaluated in young children, a large fraction of shed vaccine had lost expression of RSV F due to mutations, and immunogenicity against F was low (Yang et al., Vaccine 31:2822-2827, 2013). These examples, which all involve paramyxoviruses and pneumoviruses related to MPV, demonstrate that the properties of virus vectors expressing a supernumerary gene are often unpredictable and illustrate that the manipulation of large genomic cDNAs and recovery of genetically-stable virus free of adventitious mutations are significant challenges that must be overcome to develop an effective pneumoviruses-based vaccine.

The present disclosure overcomes these challenges to develop an MPV-based SARS-CoV-2 vaccine. The studies disclosed herein describe the safety, immunogenicity and protective efficacy in rhesus macaques of a live rMPV vector expressing the SARS-CoV-2 S protein, administered intranasally in one or two doses. In a first macaque experiment. MPV that expressed prefusion-stabilized (MPV/S-2P) or wild-type S (MPV/S) were compared. While both vaccines were safe, MPV/S-2P induced stronger systemic and mucosal anti-S antibody responses and thus was evaluated further. In a second macaque experiment, the effect of a prime/boost regimen of MPV/S-2P was assessed. While a single dose of MPV/S-2P was highly immunogenic, a second dose further increased the magnitude, breadth, avidity, and antibody-dependent cellular phagocytosis (ADCP) activity of serum anti-S antibodies that targeted the receptor binding domain (RBD) of S as well as regions outside the RBD. The second dose of MPV/S-2P also increased the magnitude and breadth of the mucosal anti-S antibodies, including dimeric anti-S IgA in the airways. MPV/S-2P also induced strong S-specific CD4⁺ and CD8⁺ T cells in the lower airways that were restimulated after boost, transitioning to tissue-resident memory phenotype. A single dose of MPV/S-2P induced strong protection against SARS-CoV-2 challenge virus replication in the airways, and two doses were fully protective. These data demonstrate that MPV/S-2P is an effective live-attenuated intranasal vaccine for immunization against SARS-CoV-2.

### II. Abbreviations

ACE2 angiotensin converting enzyme 2
ADCP antibody-dependent cellular phagocytosis
AI avidity index
BAL bronchoalveolar lavage
BSL3 biosafety level 3
COVID coronavirus infectious disease
CPE cytopathic effect
DELFIA dissociation-enhanced lanthanide fluorescence immunoassay
ELISA enzyme linked immunosorbent assay
F fusion glycoprotein
G attachment glycoprotein
gN genomic N
IC₅₀ 50% inhibitory concentration
IFN interferon
IL interleukin
IN/IT intranasal/intratracheal
L polymerase protein
LA lower airway
M matrix protein
MOI multiplicity of infection
MPV murine pneumonia virus
N nucleoprotein
NaSCN sodium thiocyanate
ND₅₀ 50% neutralization titer
NS nasopharyngeal swabs
NS1 non-structural protein 1
NS2 non-structural protein 2
NW nasal wash
ORF open reading frame
P phosphoprotein
PBMC peripheral blood mononuclear cell
PFU plaque forming unit
pc post-challenge
pi post-immunization
PRNT₆₀ 60% plaque reduction neutralization test
RBD receptor binding domain
RSV respiratory syncytial virus
RT room temperature
SARS-CoV-2 severe acute respiratory syndrome coronavirus 2
S spike
sgE subgenomic E
SH small hydrophobic protein
TCID₅₀ 50% tissue culture infectious dose
TL tracheal lavage
TMCT transmembrane/cytoplasmic tail
TNF tumor necrosis factor
TRF time resolved fluorescence
Trm tissue resident memory
UA upper airway
VoC variant of concern
WT wild type

### III. Summary of Terms

Unless otherwise noted, technical terms are used according to conventional usage. Definitions of many common terms in molecular biology may be found in Krebs er al. (eds.), Lewin's genes XII, published by Jones & Bartlett Learning, 2017. As used herein, the singular forms “a,” “an,” and “the,” refer to both the singular as well as plural, unless the context clearly indicates otherwise. For example, the term “an antigen” includes singular or plural antigens and can be considered equivalent to the phrase “at least one antigen.” As used herein, the term “comprises” means “includes.” It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described herein. In case of conflict, the present specification, including explanations of terms, will control. In addition, the materials, methods, and examples are illustrative only and not intended to be limiting. To facilitate review of the various aspects, the following explanations of terms are provided:

Adjuvant: A vehicle used to enhance antigenicity. Adjuvants can include a suspension of minerals (alum, aluminum hydroxide, or phosphate) on which antigen is adsorbed; or water-in-oil emulsion, for example, in which antigen solution is emulsified in mineral oil (Freund incomplete adjuvant), sometimes with the inclusion of killed mycobacteria (Freund's complete adjuvant) to further enhance antigenicity (inhibits degradation of antigen and/or causes influx of macrophages). Immunostimulatory oligonucleotides (such as those including a CpG motif) can also be used as adjuvants. Other adjuvants include biological molecules (a “biological adjuvant”), such as costimulatory molecules. Exemplary biological adjuvants include IL-2, RANTES, GM-CSF, TNF-α, IFN-γ, G-CSF, LFA-3, CD72, B7-1, B7-2, OX-40L, 4-1BBL, immune stimulating complex (ISCOM) matrix, and toll-like receptor (TLR) agonists, such as TLR-9 agonists, Poly I:C, or PolyICLC. Adjuvants are described, for example, in Singh (ed.) Vaccine Adjuvants and Delivery Systems; Wiley-Interscience, 2007.

Administration: The introduction of a composition into a subject by a chosen route. Administration can be local or systemic. For example, if the chosen route is intranasal, the composition (such as a composition including a disclosed rMPV or immunogenic composition thereof) is administered by introducing the composition into the nasal passages of the subject. Exemplary routes of administration include, but are not limited to, intranasal, intratracheal, oral, injection (such as subcutaneous, intramuscular, intradermal, intraperitoneal, and intravenous), sublingual, rectal, transdermal (for example, topical), intratracheal, vaginal, and inhalation routes.

Amino acid substitution: The replacement of one amino acid in a polypeptide with a different amino acid.

Codon-optimized: A nucleic acid sequence that has been altered such that the codons are optimal for expression in a particular system (such as a particular species or group of species). For example, a nucleic acid sequence can be optimized for expression in mammalian cells or in a particular mammalian species (such as human cells). Codon optimization does not alter the amino acid sequence of the encoded protein.

Codon pair optimized: A nucleic acid sequence that has been altered such that codon pairs (codons immediately 3′ or 5′ of each other) are optimized for expression in a particular system or species (such as in humans). For example, codons are altered to increase the content of codon pairs that are overrepresented in a particular system or species. In the context of the present disclosure, a portion of the MPV L ORF was codon pair optimized for efficient expression in humans and to decrease the likelihood of over-attenuation of the MPV vector backbone after insertion of a foreign gene (see Example 2). Codon pair optimization does not alter the amino acid sequence of an encoded protein.

Coronavirus: A large family of positive-sense, single-stranded RNA viruses that can infect humans and non-human animals. Coronaviruses get their name from the crown-like spikes on their surface. The viral envelope is comprised of a lipid bilayer containing the viral membrane (M), envelope (E) and spike (S) proteins. Most coronaviruses cause mild to moderate upper respiratory tract illness, such as the common cold. However, three coronaviruses have emerged that can cause more serious illness and death: severe acute respiratory syndrome coronavirus (SARS-CoV), SARS-CoV-2 (including variants thereof, such as: alpha (B.1.1.7 and Q lineages); beta (B.1.351 and descendent lineages); delta (B.1.617.2 and AY lineages); gamma (P.1 and descendent lineages); epsilon (B.1.427 and B.1.429); eta (B.1.525); iota (B.1.526); kappa (B.1.617.1); 1.617.3; mu (B.1.621, B.1.621.1), zeta (P.2), and omicron (B.1.1.529, BA.1, BA.1.1, BA.2, BA.2.75, BA.2.86, BA.3, BA.4, BA.5, CH.1.1, XBB, XBB.1.5, XBB.2.3, XBB.1.9.1, XBB.1.9.2, XBB.1.16, BA.2.86 and BQ.1 lineages)), and Middle East respiratory syndrome coronavirus (MERS-CoV). Other coronaviruses that infect humans include human coronavirus HKU1 (HKU1-CoV), human coronavirus OC43 (OC43-CoV), human coronavirus 229E (229E-CoV), human coronavirus NL63 (NL63-CoV).

COVID or COVID-19: The disease caused by the coronavirus SARS-CoV-2.

Degenerate variant: A polynucleotide encoding a polypeptide that includes a sequence that is degenerate as a result of the genetic code. There are 20 natural amino acids, most of which are specified by more than one codon. Therefore, all degenerate nucleotide sequences encoding a peptide are included as long as the amino acid sequence of the peptide encoded by the nucleotide sequence is unchanged.

Effective amount: An amount of agent, such as a rMPV or immunogenic composition thereof, as described herein, that is sufficient to elicit a desired response, such as an immune response in a subject. It is understood that obtaining a protective immune response against an antigen of interest can require multiple administrations of a disclosed immunogen, and/or administration of a disclosed immunogen as the “prime” in a prime boost protocol wherein the boost immunogen can be the same or different from the prime immunogen. Accordingly, an effective amount of a disclosed immunogen can be the amount of the immunogen sufficient to elicit a priming immune response in a subject that can be subsequently boosted with the same or a different immunogen to elicit a protective immune response. A “therapeutically effective amount” can be the amount necessary to inhibit SARS-CoV-2 replication or treat COVID-19 in a subject with an existing SARS-CoV-2 infection. A “prophylactically effective amount” refers to administration of an agent or composition that inhibits or prevents establishment of an infection, such infection by SARS-CoV-2.

In one example, a desired response is to inhibit or reduce or prevent SARS-CoV-2 infection or associated disease. The SARS-CoV-2 infection does not need to be completely eliminated or reduced or prevented for the method to be effective. For example, administration of an effective amount of an immunogenic composition disclosed herein can induce an immune response that decreases the SARS-CoV-2 infection (for example, as measured by infection of cells, or by number or percentage of subjects infected by the SARS-CoV-2) by a desired amount, for example by at least 50%, at least 60%, at least 70%, at least 80%, at least 90%, at least 95%, at least 98%, or even at least 100% (elimination or prevention of detectable SARS-CoV-2 infection), as compared to a suitable control.

Gene: A nucleic acid sequence that comprises control and coding sequences necessary for the transcription of an RNA, whether an mRNA or otherwise. For instance, a gene may comprise a promoter, one or more enhancers or silencers, a nucleic acid sequence that encodes a RNA and/or a polypeptide, downstream regulatory sequences and, possibly, other nucleic acid sequences involved in regulation of the expression of an mRNA. A “gene” of a rMPV as described herein refers to a portion of the MPV genome encoding an RNA. An MPV gene typically begins at the upstream (3′) end with a gene-start (GS) signal and ends at the downstream (5′) end with the gene-end (GE) signal. In this context, the term “gene” also embraces what is referred to as a “translational open reading frame” (or ORF).

Heterologous: Originating from a different genetic source. A heterologous gene included in a recombinant genome is a gene that does not originate from that genome. In one specific, non-limiting example, a heterologous gene encoding a recombinant SARS-CoV-2 S protein is included in the genome of a rMPV as described herein.

Host cells: Cells in which a vector can be propagated, and its nucleic acid expressed. The cell may be prokaryotic or eukaryotic. The term also includes any progeny of the subject host cell. It is understood that all progeny may not be identical to the parental cell since there may be mutations that occur during replication. However, such progeny are included when the term “host cell” is used. In some aspects herein, the host cell is a cell in which rMPV can be propagated. In some examples, the host cell expresses a T7 RNA polymerase (e.g., the cells are BSR-T7/5 cells).

Immune response: A response of a cell of the immune system, such as a B cell, T cell, or monocyte, to a stimulus. In one aspect, the response is specific for a particular antigen (an “antigen-specific response”). In one aspect, an immune response is a T cell response, such as a CD4+ response or a CD8+ response. In another aspect, the response is a B cell response, and results in the production of specific antibodies. A “protective immune response” is an immune response sufficient to prevent or inhibit infection by a particular pathogen, such as SARS-CoV-2. The protective immune response need not provide 100% protection against infection. In one example, a desired response is to inhibit or reduce or prevent SARS-CoV-2 infection or associated disease. The SARS-CoV-2 infection does not need to be completely eliminated or reduced or prevented for the method to be effective. For example, administration of an effective amount of an immunogenic composition disclosed herein can induce an immune response (such as a protective immune response) that decreases the SARS-CoV-2 infection (for example, as measured by infection of cells, or by number or percentage of subjects infected by the SARS-CoV-2) by a desired amount, for example by at least 80%, at least 90%, at least 95%, at least 98%, or even at least 100% (elimination or prevention of detectable SARS-CoV-2 infection), as compared to a suitable control.

Immunogenic composition: A preparation of immunogenic material capable of stimulating an immune response, which in some examples can be administered for the prevention, amelioration, or treatment of infectious (such as SARS-CoV-2 infection) or other types of disease. The immunogenic material may include attenuated or killed microorganisms (such as bacteria or viruses), or antigenic proteins, peptides or DNA derived from them. Immunogenic compositions comprise an antigen (such as a virus) that induces a measurable T cell response against the antigen or induces a measurable B cell response (such as production of antibodies) against the antigen. In some aspects herein, an immunogenic composition includes a disclosed rMPV that induces a measurable CTL response against SARS-CoV-2 S protein or induces a measurable B cell response (such as production of antibodies) against SARS-CoV-2 S protein, when administered to a subject. For in vivo use, the immunogenic composition will typically include a recombinant virus in a pharmaceutically acceptable carrier and may also include other agents, such as an adjuvant.

Isolated: An “isolated” biological component has been substantially separated or purified away from other biological components, such as other biological components in which the component occurs, such as other chromosomal and extrachromosomal DNA, RNA, and proteins. Proteins, peptides, nucleic acids, and viruses that have been “isolated” include those purified by standard purification methods. Isolated does not require absolute purity, and can include protein, peptide, nucleic acid, or virus particles that are at least 50% pure, such as at least 75%, 80%, 90%, 95%, 98%, 99%, or even 99.9% pure.

Live-attenuated virus: A virus capable of infecting and replicating in a host, and inducing an immune response in the host, but is not pathogenic or minimally pathogenic. In some instances, an attenuated virus is a virus modified to render it attenuated. Alternatively, some viruses are naturally attenuated in particular hosts, such as in humans or non-human primates. In the context of the present disclosure, MPV (and recombinant versions thereof) is capable of infecting non-human primates and human subjects and inducing an immune response but does not cause disease.

Murine pneumonia virus (MPV): An enveloped, single-stranded negative-sense RNA virus in the genus Pneumovirus, family Paramyxoviridae. MPV is related to the human virus respiratory syncytial virus (RSV). The genome of MPV is approximately 15 kb and includes, in a 3′ to 5′ order, a 3′ leader sequence, an MPV non-structural protein 1 (NS1) gene, an MPV non-structural protein 2 (NS2) gene, a heterologous gene, an MPV nucleoprotein (N) gene, an MPV phosphoprotein (P) gene, an MPV matrix protein (M) gene, an MPV small hydrophobic protein (SH) gene, an MPV attachment glycoprotein (G) gene, an MPV fusion glycoprotein (F) gene, an MPV M2 gene, an MPV polymerase protein (L) gene, and a 5′ trailer sequence. MPV genes include gene start (GS) and gene end (GE) transcription signals. As used herein, an MPV “antigenome” is a nucleic acid molecule (such as a cDNA) transcribed from the negative-sense RNA genome. MPV naturally infects mice, rats, hamsters, guinea pigs, and other rodents. Although MPV can infect humans and non-human primates, it is naturally attenuated in these species.

Nucleic acid molecule: A polymeric form of nucleotides, which may include both sense and anti-sense strands of RNA, cDNA, genomic DNA, and synthetic forms and mixed polymers of the above. A nucleotide refers to a ribonucleotide, deoxynucleotide or a modified form of either type of nucleotide. The term “nucleic acid molecule” as used herein is synonymous with “polynucleotide.” A nucleic acid molecule is usually at least 10 bases in length, unless otherwise specified. The term includes single- and double-stranded forms of DNA. A nucleic acid molecule may include either or both naturally occurring and modified nucleotides linked together by naturally occurring and/or non-naturally occurring nucleotide linkages.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked nucleic acid sequences are contiguous and, where necessary to join two protein-coding regions, in the same reading frame.

Preventing, treating or ameliorating a disease: “Preventing” a disease refers to inhibiting the full development of a disease. “Treating” refers to a therapeutic intervention that ameliorates a sign or symptom of a disease or pathological condition after it has begun to develop, such as a reduction in viral load. “Ameliorating” refers to the reduction in the number or severity of signs or symptoms of a disease, such as a coronavirus infection.

Pharmaceutically acceptable carriers: The pharmaceutically acceptable carriers of use are conventional. Remington: The Science and Practice of Pharmacy, 22_{nd} ed., London, UK: Pharmaceutical Press, 2013, describes compositions and formulations suitable for pharmaceutical delivery of the disclosed immunogens.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate. In particular aspects, suitable for administration to a subject the carrier may be sterile, and/or suspended or otherwise contained in a unit dosage form containing one or more measured doses of the composition suitable to induce the desired immune response. It may also be accompanied by medications for its use for treatment purposes. The unit dosage form may be, for example, in a sealed vial that contains sterile contents or a syringe for injection into a subject, or lyophilized for subsequent solubilization and administration or in a solid or controlled release dosage.

Recombinant: A recombinant nucleic acid, vector or virus is one that has a sequence that is not naturally occurring or has a sequence that is made by an artificial combination of two otherwise separated segments of sequence. This artificial combination can be accomplished, for example, by the artificial manipulation of isolated segments of nucleic acids, for example, using genetic engineering techniques.

SARS-CoV-2: A coronavirus of the genus betacoronavirus that first emerged in humans in 2019. This virus is also known as Wuhan coronavirus, 2019-nCoV, or 2019 novel coronavirus. The term “SARS-CoV-2” includes variants thereof, such as, but not limited to, alpha (13.1.1.7 and Q lineages); beta (B.1.351 and descendent lineages); delta (B.1.617.2 and AY lineages); gamma (P.1 and descendent lineages); epsilon (B.1.427 and B.1.429); eta (13.1.525); iota (13.1.526); kappa (B.1.617.1); 1.617.3; mu (13.1.621, B.1.621.1), zeta (P.2) and omicron (B.1.1.529, BA.1, BA.1.1, BA.2, BA.2.75, BA.2.86, BA.3, BA.4, BA.5, CH.1.1, XBB, XBB.1.5, XBB.2.3, XBB.1.9.1, XBB.1.9.2, XBB.1.16, BA.2.86 and BQ.1 lineages). Symptoms of SARS-CoV-2 infection include fever, chills, dry cough, shortness of breath, fatigue, muscle/body aches, headache, new loss of taste or smell, sore throat, nausea or vomiting, and diarrhea. Patients with severe disease can develop pneumonia, multi-organ failure, and death. The time from exposure to onset of symptoms is approximately 2 to 14 days. The SARS-CoV-2 virion includes a viral envelope with large spike glycoproteins. The SARS-CoV-2 genome, like most coronaviruses, has a common genome organization with the replicase gene included in the 5′-two thirds of the genome, and structural genes included in the 3-third of the genome. The SARS-CoV-2 genome encodes the canonical set of structural protein genes in the order 5′-spike (S)-envelope (E)-membrane (M) and nucleocapsid (N)-3′.

Standard methods for detecting viral infection may be used to detect SARS-CoV-2 infection, including but not limited to, assessment of patient symptoms and background and genetic tests such as reverse transcription-polymerase chain reaction (rRT-PCR). The test can be done on patient samples such as respiratory or blood samples.

SARS-CoV-2 Spike (S): A class I fusion glycoprotein initially synthesized as a precursor protein of approximately 1270 amino acids in size. Individual precursor S polypeptides form a homotrimer and undergo glycosylation within the Golgi apparatus as well as processing to remove the signal peptide. The S polypeptide includes S1 and S2 proteins separated by a protease cleavage site between approximately position 685/686. Cleavage at this site generates separate S1 and S2 polypeptide chains, which remain associated as S1/S2 protomers within the homotrimer. It is believed that the beta coronaviruses are generally not cleaved prior to the low pH cleavage that occurs in the late endosome-early lysosome by the transmembrane protease serine 2 (TMPRSS2), at an additional proteolytic cleavage site S2/S2′ at the start of the fusion peptide. Cleavage between S1/S2 is not required for function and is not observed in all viral spikes. The S1 subunit is distal to the virus membrane and contains the receptor-binding domain (RBD) that is believed to mediate virus attachment to its host receptor. The S2 subunit is believed to contain the fusion protein machinery, such as the fusion peptide, two heptad-repeat sequences (HR1 and HR2) and a central helix typical of fusion glycoproteins, a transmembrane domain, and the cytosolic tail domain. The numbering used in the disclosed SARS-CoV-2 S proteins and fragments thereof is relative to the wild-type S protein of the Wuhan-1 strain of SARS-CoV-2 set forth herein as SEQ ID NO: 33.

Sequence identity: The similarity between amino acid sequences is expressed in terms of the similarity between the sequences, otherwise referred to as sequence identity. Sequence identity is frequently measured in terms of percentage identity (or similarity or homology); the higher the percentage, the more similar the two sequences are. Homologs, orthologs, or variants of a polypeptide will possess a relatively high degree of sequence identity when aligned using standard methods.

When determining sequence identity between two sequences, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Optimal alignment of sequences for comparison can be conducted, e.g., by the local homology algorithm of Smith & Waterman, Adv. Appl. Math. 2:482, 1981, by the homology alignment algorithm of Needleman & Wunsch, J. Mol. Biol. 48:443, 1970, by the search for similarity method of Pearson & Lipman, Proc. Nat'l. Acad. Sci. USA 85:2444, 1988, by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see, e.g., Sambrook et al. (Molecular Cloning: A Laboratory Manual, 4^{th} ed, Cold Spring Harbor, New York, 2012) and Ausubel et al. (In Current Protocols in Molecular Biology, John Wiley & Sons, New York, through supplement 104, 2013).

Another example of algorithms that are suitable for determining percent sequence identity and sequence similarity are the BLAST and the BLAST 2.0 algorithm, which are described in Altschul et al., J. Mol. Biol. 215:403-410, 1990 and Altschul et al., Nucleic Acids Res. 25:3389-3402, 1977. Software for performing BLAST analyses is publicly available through the National Center for Biotechnology Information (ncbi.nlm.nih.gov). The BLASTN program (for nucleotide sequences) uses as defaults a word length (W) of 11, alignments (B) of 50, expectation (E) of 10, M=5, N=−4, and a comparison of both strands. The BLASTP program (for amino acid sequences) uses as defaults a word length (W) of 3, and expectation (E) of 10, and the BLOSUM62 scoring matrix (see Henikoff & Henikoff, Proc. Natl. Acad. Sci. USA 89:10915, 1989).

Homologs and variants of a polypeptide (such as a SARS-CoV-2 S protein) are typically characterized by possession of at least about 75%, for example at least about 80%, 85%, 90%, 91%, 92%, 93%, 94%, 95%, 96%, 97%, 98% or 99% sequence identity counted over the full-length alignment with the amino acid sequence of interest. As used herein, reference to “at least 90% identity” or similar language refers to “at least 90%, at least 91%, at least 92%, at least 93%, at least 94%, at least 95%, at least 96%, at least 97%, at least 98%, at least 99%, or even 100% identity” to a specified reference sequence.

Subject: Living multi-cellular vertebrate organisms, a category that includes human and non-human mammals, including non-human primates. In some examples, the subject is a human subject. In particular examples, the subject is at risk of SARS-CoV-2 infection.

Vaccine: A preparation of immunogenic material capable of stimulating an immune response, administered for the prevention, amelioration, or treatment of infectious or other types of disease. The immunogenic material may include attenuated or killed microorganisms (such as bacteria or viruses), or antigenic proteins, peptides or DNA derived from them. Vaccines may elicit both prophylactic (preventative or protective) and therapeutic responses. Methods of administration vary according to the vaccine, but may include inoculation, ingestion, inhalation or other forms of administration. Vaccines may be administered with an adjuvant to boost the immune response.

Vector: An entity containing a DNA or RNA molecule bearing a promoter(s) that is operationally linked to the coding sequence of an antigen(s) of interest and can express the coding sequence. Non-limiting examples include a naked or packaged (lipid and/or protein) DNA, a naked or packaged RNA, a subcomponent of a virus or bacterium or other microorganism that may be replication-incompetent, or a virus or bacterium or other microorganism that may be replication-competent. A vector is sometimes referred to as a construct. Recombinant DNA vectors are vectors having recombinant DNA. A vector can include nucleic acid sequences that permit it to replicate in a host cell, such as an origin of replication. A vector can also include one or more selectable marker genes and other genetic elements known in the art. Viral vectors are recombinant nucleic acid vectors having at least some nucleic acid sequences derived from one or more viruses. In some examples herein, the vector is a murine pneumonia virus (MPV) vector.

### IV. Recombinant Murine Pneumonia Virus (MPV) Expressing SARS-CoV-2 Spike Protein

SARS-CoV-2 vaccines that induce immunity in the airways ale needed to protect against SARS-CoV-2 infection and replication. The present disclosure evaluated the immunogenicity and protective efficacy of a live-attenuated recombinant murine pneumonia virus (rMPV) vector expressing SARS-CoV-2 prefusion stabilized spike protein (MPV/S-2P), delivered intranasally/intratracheally to rhesus macaques. The results demonstrated that a single dose of MPV/S-2P was highly immunogenic, and a second dose further increased the magnitude and breadth of the mucosal and systemic anti-S antibody response and increased levels of dimeric anti-S IgA in the airways. MPV/S-2P also induced mucosal S-specific CD4⁺ and CD8⁺ T cells that were restimulated after boost while transitioning to tissue-resident memory cells. One dose induced substantial protection against SARS-CoV-2 challenge, and two doses of MPV/S-2P were fully protective against SARS-CoV-2 challenge virus replication in the airways, indicating that prime/boost immunization with a mucosally-administered live-attenuated MPV vector would be highly effective in preventing SARS-CoV-2 infection and replication.

Provided herein are live-attenuated recombinant murine pneumonia viruses (rMPV) that express a SARS-CoV-2 S protein from a heterologous ORF inserted in the MPV genome. In some aspects, the rMPV has a genome that includes in a 3′ to 5′ order, a 3′ leader sequence, an MPV non-structural protein 1 (NS1) gene, an MPV non-structural protein 2 (NS2) gene, a heterologous gene encoding a SARS-CoV-2 S protein, an MPV nucleoprotein (N) gene, an MPV phosphoprotein (P) gene, an MPV matrix protein (M) gene, an MPV small hydrophobic protein (SH) gene, an MPV attachment glycoprotein (G) gene, an MPV fusion glycoprotein (F) gene, an MPV M2 gene, an MPV polymerase protein (L) gene, and a 5′ trailer sequence.

In other aspects, the rMPV has a genome that includes in a 3′ to 5′ order, a 3′ leader sequence, an MPV NS1 gene, an MPV NS2 gene, an MPV N gene, a heterologous gene encoding a SARS-CoV-2 S protein, an MPV P gene, an MPV M gene, an MPV SH gene, an MPV G gene, an MPV F gene, an MPV M2 gene, an MPV L gene, and a 5′ trailer sequence.

In some aspects, the heterologous gene encodes a SARS-CoV-2 S protein that is stabilized in the prefusion conformation. In particular examples, the stabilized S protein includes 2 proline substitutions (such as at positions 986 and 987; referred to as “S-2P”) or six proline substitutions (such as at positions 817, 892, 899, 942, 986 and 987; referred to as “S-6P”), with respect to the WT S protein sequence of the Wuhan-1 strain set forth herein as SEQ ID NO: 33.

In some aspects, the heterologous gene encodes a SARS-CoV-2 S protein that has one or more substitutions or deletions to inactivate or mutate a polybasic furin cleavage motif. In some examples, the S protein includes R682G, R683S and R685S amino acid substitutions (also referred to as “RRAR to GSAS” at positions 682 to 685, with reference to SEQ ID NO: 33).

In some examples, the S protein includes K986P and V987P amino acid substitutions, numbered with reference to SEQ ID NO: 33. In specific examples, the S protein further includes F817P, A892P, A899P, and A942P amino acid substitutions and/or R682G, R683S, R685S substitutions, numbered with reference to SEQ ID NO: 33. In other specific examples, the S protein includes R682G, R683S, R685S, K986P, V987P and F446L amino acid substitutions, numbered with reference to SEQ ID NO: 33.

In some aspects of the rMPV disclosed herein, the heterologous gene is flanked by MPV gene start (GS) and gene end (GE) transcription signals.

In some aspects, the S protein open reading frame (ORF) is transcribed by the MPV polymerase complex as a distinct mRNA.

In some aspects, the heterologous gene is codon-optimized for expression in human cells.

In some aspects, a Kozak consensus sequence (GCCGCCACC) or a modified Kozak consensus sequence with reduced G/C content is inserted immediately upstream of the heterologous gene start codon.

In some aspects, an adenosine residue is inserted three nucleotides upstream (−3) of the heterologous gene start codon.

In some aspects, the MPV L gene is codon pair optimized for expression in human cells.

In some aspects, the amino acid sequence of the S protein is at least 80%, at least 85%, at least 90%, at least 95%, at least 96%, at least 97%, at least 98%, or at least 99% identical to SEQ ID NO: 33. SEQ ID NO: 34, SEQ ID NO: 36, SEQ ID NO: 37, SEQ ID NO: 38, SEQ ID NO: 39, SEQ ID NO: 54, or SEQ ID NO: 55. In some examples, the amino acid sequence of the S protein comprises or consists of SEQ ID NO: 33, SEQ ID NO: 34, SEQ ID NO: 36, SEQ ID NO: 37, SEQ ID NO: 38, SEQ ID NO: 39, SEQ ID NO: 54 or SEQ ID NO: 55.

In some aspects, the genome of the rMPV includes an antigenomic cDNA sequence at least 80%, at least 85%, at least 90%, at least 95%, at least 96%, at least 97%, at least 98%, or at least 99% identical to SEQ ID NO: 9, SEQ ID NO: 11, SEQ ID NO: 25, SEQ ID NO: 27, SEQ ID NO: 29, SEQ ID NO: 31, SEQ ID NO: 50, or SEQ ID NO: 52. In some examples, the genome includes an antigenomic cDNA sequence comprising or consisting of SEQ ID NO: 9, SEQ ID NO: 11, SEQ ID NO: 25, SEQ ID NO: 27, SEQ ID NO: 29, SEQ ID NO: 31, SEQ ID NO: 50, or SEQ ID NO: 52.

Also provided herein are nucleic acid molecules that include the nucleotide sequence of the genome of a rMPV disclosed herein, or an antigenomic cDNA or RNA sequence of the genome.

Further provided are vectors that include a nucleic acid molecule disclosed herein. In some aspects, the vector further includes a promoter operably linked to the nucleic acid molecule. In some examples, the promoter is a T7 promoter.

In some aspects of the disclosed vectors, the vector further includes a ribozyme sequence and a transcription terminator operably linked to the nucleic acid molecule. In some examples, the last nucleotide of the 5′ trailer sequence is followed by three guanosine residues, a hepatitis delta virus ribozyme sequence, and a T7 terminator.

In some examples, the nucleotide sequence of the vector is at least 80%, at least 85%, at least 90%, at least 95%, at least 96%, at least 97%, at least 98%, or at least 99% identical to SEQ ID NO: 10, SEQ ID NO: 12, SEQ ID NO: 26, SEQ ID NO: 28, SEQ ID NO: 30, SEQ ID NO: 32, SEQ ID NO: 51 or SEQ ID NO: 53. In specific examples, the nucleotide sequence of the vector comprises or consists of SEQ ID NO: 10, SEQ ID NO: 12, SEQ ID NO: 26, SEQ ID NO: 28, SEQ ID NO: 30, SEQ ID NO: 32, SEQ ID NO: 51 or SEQ ID NO: 53.

Further provided herein are isolated host cells that include a nucleic acid molecule or vector disclosed herein. In some aspects, the host cell is a mammalian cell, such as a bovine hamster kidney (BHK) cell. In other aspects, the host cell is a prokaryotic cell.

Also provided herein are in vitro methods of producing a rMPV. In some aspects, the method includes transfecting MPV-permissive cells with a vector disclosed herein, and plasmids encoding MPV N, P, L and M2-1 proteins; incubating the cell culture for a period of time sufficient to allow for viral replication; and purifying the replicated virus to produce the rMPV. In some examples, the MPV-permissive cells express T7 RNA polymerase, or the MPV-permissive cells are transfected with a plasmid encoding T7 RNA polymerase. Further provided are rMPV produced by the methods disclosed herein.

Also provided herein are immunogenic compositions that include a pharmaceutically acceptable carrier and a rMPV disclosed herein. In some aspects, the immunogenic composition further includes an adjuvant. In some examples, the immunogenic composition includes or further includes a secondary therapeutic agent, such as but not limited to, a decongestant, an antihistamine, a pain reliever, a fever reducer, a cough suppressant, a cytokine, an antibiotic, an anti-viral agent, or any combination thereof. In some aspects of the immunogenic composition, the composition is formulated for intranasal administration. In some examples, the composition is formulated as a spray, mist, or aerosol.

Further provided herein are methods of eliciting an immune response against SARS-CoV-2 in a subject. In some aspects, the method includes administering to the subject an effective amount of a rMPV, nucleic acid molecule, vector, or immunogenic composition disclosed herein. In particular aspects, the method includes administering an immunogenic composition disclosed herein. In some examples, the immunogenic composition is administered intranasally, such as in the form of a spray, mist, or aerosol.

In some aspects, the rMPV, nucleic acid molecule, vector, or immunogenic composition is administered in a single dose. In other aspects, the rMPV, nucleic acid molecule, vector, or immunogenic composition is administered in two doses (for example, prime and boost doses).

In some aspects of the disclosed methods, the subject is a human subject.

In some aspects, the immune response elicited in response to administration of a disclosed rMPV, nucleic acid molecule, vector, or immunogenic composition is a protective immune response.

In some aspects, the immunogenic composition is administered as a prime dose in a prime-boost dosing regimen. In other aspects, the immunogenic composition is administered as a boost dose in a prime-boost dosing regimen. In yet other aspects, the immunogenic composition is administered as both the prime and boost doses in a prime-boost dosing regimen.

Use of a disclosed rMPV, nucleic acid molecule, vector, or immunogenic composition for eliciting an immune response against SARS-CoV-2 in a subject is also provided. In some examples, provided is the use of an immunogenic composition disclosed herein for eliciting an immune response against SARS-CoV-2 in a subject.

### V. Immunogenic Compositions

Immunogenic compositions that include a rMPV expressing a SARS-CoV-2 S protein, as disclosed herein, and a pharmaceutically acceptable carrier are also provided. Such compositions can be administered to a subject by a variety of modes, for example, by intranasal, onto the tonsils, inhalation, oral, intramuscular, subcutaneous, intravenous, intra-arterial, intra-articular, intraperitoneal, or parenteral routes. In particular examples, the immunogenic composition is administered intranasally. Standard methods for preparing administrable immunogenic compositions are described, for example, in such publications as Remington: The Science and Practice of Pharmacy, 22^{nd} ed., London, UK: Pharmaceutical Press, 2013.

Thus, an immunogen described herein can be formulated with pharmaceutically acceptable carriers to help retain biological activity while also promoting increased stability during storage within an acceptable temperature range. Potential carriers include, but are not limited to, physiologically balanced culture medium, phosphate buffered saline solution, water, emulsions (e.g., oil/water or water/oil emulsions), various types of wetting agents, cryoprotective additives or stabilizers such as proteins, peptides or hydrolysates (e.g., albumin, gelatin), sugars (e.g., sucrose, lactose, sorbitol), amino acids (e.g., sodium glutamate), or other protective agents. The resulting aqueous solutions may be packaged for use as is or lyophilized. Lyophilized preparations are combined with a sterile solution prior to administration for either single or multiple dosing.

The immunogenic composition can contain a bacteriostat to prevent or minimize degradation during storage, including but not limited to effective concentrations (usually ≤1% w/v) of benzyl alcohol, phenol, m-cresol, chlorobutanol, methylparaben, and/or propylparaben. A bacteriostat may be contraindicated for some patients; therefore, a lyophilized formulation may be reconstituted in a solution either containing or not containing such a component.

The immunogenic composition can contain as pharmaceutically acceptable vehicles substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, and triethanolamine oleate.

The immunogenic composition may optionally include an adjuvant to enhance the immune response of the host. Suitable adjuvants are, for example, toll-like receptor agonists, alum, AlPO₄, alhydrogel, Lipid-A and derivatives or variants thereof, oil-emulsions, saponins, neutral liposomes, liposomes, and cytokines, non-ionic block copolymers, and chemokines. Non-ionic block polymers containing polyoxyethylene (POE) and polyxylpropylene (POP), such as POE-POP-POE block copolymers, MPL™ (3-O-deacylated monophosphoryl lipid A; Corixa, Hamilton, IN) and IL-12 (Genetics Institute, Cambridge, MA), among many other suitable, well-known adjuvants may be used as an adjuvant (Newman et al., 1998, Critical Reviews in Therapeutic Drug Carrier Systems 15:89-142). These adjuvants help to stimulate the immune system in a non-specific way, thus enhancing the immune response to a pharmaceutical product. In some aspects, an adjuvant is not required and is thus not administered with the immunogenic composition containing rMPV.

In some instances, it may be desirable to combine the immunogenic composition including the rMPV with other pharmaceutical products (e.g., vaccines) which induce protective responses to other viral agents, such as those causing childhood illnesses. For example, a composition including a rMPV as described herein can also include other vaccines recommended by the Advisory Committee on Immunization Practices (ACIP; cdc.gov/vaccines/acip) for the targeted age group. These additional vaccines include, but are not limited to, intranasally administered vaccines. As such, a rMPV as described herein may be administered simultaneously with vaccines against, for example, hepatitis B (HepB) virus, diphtheria, tetanus and pertussis (DTaP), pneumococcal bacteria (PCV), Haemophilus influenzae type b (Hib), poliovirus, influenza virus, respiratory syncytial virus (RSV), or rotavirus.

In some aspects, the immunogenic composition is provided as a sterile composition. The composition typically contains an effective amount of a disclosed immunogen and can be prepared by conventional techniques. Typically, the amount of immunogen in each dose of the immunogenic composition is selected as an amount which elicits an immune response without significant, adverse side effects. In all subjects, the precise amount of rMPV administered and the timing and repetition of administration will be determined by various factors, including the patient's state of health and weight, the mode of administration, the nature of the formulation, etc. Dosages will generally range from about 3.0 log₁₀ to about 7.0 log₁₀ plaque forming units (PFU) or more of virus per patient, such as about 3.0 log₁₀, 3.5 log₁₀, 4.0 log₁₀, 4.5 log₁₀, 5.0 log₁₀ 5.5 log₁₀, 6.0 log₁₀, 6.5 log₁₀ or 7.0 log₁₀ PFU virus per patient.

In some aspects, the immunogenic composition can be provided in unit dosage form to induce an immune response in a subject, for example, to prevent SARS-CoV-2 infection in the subject. A unit dosage form contains a suitable single preselected dosage for administration to a subject, or suitable marked or measured multiples of two or more preselected unit dosages, and/or a metering mechanism for administering the unit dose or multiples thereof.

### VI. Nucleic Acid Molecules and Vectors

Nucleic acid molecules and vectors encoding a disclosed immunogen (e.g., a rMPV encoding a SARS-CoV-2 S protein, or a plasmid encoding a full-length clone of a rMPV) are provided. In some aspects, the vector is a viral vector (e.g., rMPV), or the vector is a plasmid vector. These nucleic acid molecules include DNA, cDNA (such as an MPV antigenomic cDNA), and RNA sequences. One of skill in the art can readily use the genetic code to construct a variety of functionally equivalent nucleic acids, such as nucleic acids that differ in sequence, but which encode the same protein sequence (such as encode a SARS-CoV-2 S protein or a MPV protein).

In some aspects, the nucleic acid molecule is a rMPV genomic or antigenomic sequence, such as a cDNA sequence, that includes a heterologous ORF encoding a SARS-CoV-2 S protein. In some examples, the nucleic acid molecule includes the nucleotide sequence of rMPV/NS2N/S set forth as SEQ ID NO: 5, the nucleotide sequence of rMPV/NS2N/S (GSopt) set forth as SEQ ID NO: 7, the nucleotide sequence of rMPV/NS2N/S-2P set forth as SEQ ID NO: 9, the nucleotide sequence of rMPV/NS2N/S-2P (GSopt) set forth as SEQ ID NO: 11, the nucleotide sequence of rMPV/NS2N/S-TMCT (GSopt) set forth as SEQ ID NO: 13, the nucleotide sequence of rMPV/NP/S (WT) set forth as SEQ ID NO: 15, the nucleotide sequence of rMPV/NP/S (GSopt) set forth as SEQ ID NO: 17, the nucleotide sequence of rMPV/NP/S-2P set forth as SEQ ID NO: 19, the nucleotide sequence of rMPV/NP/S-2P (GSopt) set forth as SEQ ID NO: 21, the nucleotide sequence of rMPV/NP/S-TMCT (GSopt) set forth as SEQ ID NO: 23, the nucleotide sequence of rMPV/S-6P/B.1.617.2 Delta (GSopt) set forth as SEQ ID NO: 25, the nucleotide sequence of rMPV/S-6P/B.1.1.529 Omicron (GSopt) set forth as SEQ ID NO: 27, the nucleotide sequence of rMPV/S-2P/BA.5 Omicron (GSopt) set forth as SEQ ID NO: 29, the nucleotide sequence of rMPV/S-6P (GSopt), set forth as SEQ ID NO: 31, the nucleotide sequence of rMPV/S-2P/XBB.1.5 (GSopt) set forth as SEQ ID NO: 50, the nucleotide sequence of rMPV/S-2P/XBB.1.5/F456L (GSopt) set forth as SEQ ID NO: 52, or a nucleotide sequence at least 80%, at least 85%, at least 90%, at least 95%, at least 96%, at least 97%, at least 98%, or at least 99% identical to any one of SEQ ID NOs: 5, 7, 9, 11, 13, 15, 17, 19, 21, 23, 25, 27, 29, 31, 50 and 52. In certain examples, the nucleotide sequence of the nucleic acid molecule or vector consists of any one of SEQ ID NOs: 5, 7, 9, 11, 13, 15, 17, 19, 21, 23, 25, 27, 29, 31, 50 and 52.

In other aspects, the nucleic acid molecule is a plasmid containing a full-length rMPV clone that includes a heterologous ORF encoding a SARS-CoV-2 S protein. In some examples, the nucleic acid molecule includes the nucleotide sequence of pMPV/NS2N/S set forth as SEQ ID NO: 6, the nucleotide sequence of pMPV/NS2N/S (GSopt) set forth as SEQ ID NO: 8, the nucleotide sequence of pMPV/NS2N/S-2P set forth as SEQ ID NO: 10, the nucleotide sequence of pMPV/NS2N/S-2P (GSopt) set forth as SEQ ID NO: 12, the nucleotide sequence of pMPV/NS2N/S-TMCT (GSopt) set forth as SEQ ID NO: 14, the nucleotide sequence of pMPV/NP/S (WT) set forth as SEQ ID NO: 16, the nucleotide sequence of pMPV/NP/S (GSopt) set forth as SEQ ID NO: 18, the nucleotide sequence of prMPV/NP/S-2P set forth as SEQ ID NO: 20, the nucleotide sequence of pMPV/NP/S-2P (GSopt) set forth as SEQ ID NO: 22, the nucleotide sequence of pMPV/NP/S-TMCT (GSopt) set forth as SEQ ID NO: 24, the nucleotide sequence of pMPV/S-6P/B.1.617.2 Delta (GSopt) set forth as SEQ ID NO: 26, the nucleotide sequence of pMPV/S-6P/B.1.1.529 Omicron (GSopt) set forth as SEQ ID NO: 28, the nucleotide sequence of pMPV/S-2P/BA.5 Omicron (GSopt) set forth as SEQ ID NO: 30, the nucleotide sequence of pMPV/S-6P (GSopt), set forth as SEQ ID NO: 32, the nucleotide sequence of pMPV/S-2P-XBB.1.5 (GSopt) set forth as SEQ ID NO: 51, the nucleotide sequence of pMPV/S-2P/XBB.1.5/F456L (GSopt) set forth as SEQ ID NO: 53, or a nucleotide sequence at least 80%, at least 85%, at least 90%, at least 95%, at least 96%, at least 97%, at least 98%, or at least 99% identical to any one of SEQ ID NOs: 6, 8, 10, 12, 14, 16, 18, 20, 22, 24, 26, 28, 30, 32, 51 and 53. In certain examples, the nucleotide sequence of the nucleic acid molecule or vector consists of any one of SEQ ID NOs: 6, 8, 10, 12, 14, 16, 18, 20, 22, 24, 26, 28, 30, 32, 51 and 53.

In some aspects, the rMPV genome includes a deletion, in whole or in part, of one or more of the NS1, NS2, G and SH genes. Previous studies showed that rMPV bearing a deletion of the NS1, NS2 and G genes remained infectious and replication-competent (Krempl et al., J Virol 81(17):9490-9501, 2007; Buchholz et al., J Virol 83(4):1969-1680, 2009). Other studies showed that the SH gene can be deleted from the pneumoviruses RSV and HMPV, and paramyxoviruses such as parainfluenza virus type 5 and mumps virus, with little to no significant effect on infectivity and replication-competence. Thus, in some examples, the rMPV disclosed herein lacks any one or more of the NS1, NS2, G, and SH genes, in whole or in part, while retaining the ability to be used as a live-attenuated vector.

In some aspects, the disclosed nucleic acid molecules encoding an immunogen (e.g., rMPV) are operatively linked to one or more expression control sequences. An expression control sequence operatively linked to a coding sequence is ligated such that expression of the coding sequence is achieved under conditions compatible with the expression control sequences. The expression control sequences can include, but are not limited to, appropriate promoters, enhancers, transcription terminators, a start codon (i.e., ATG) in front of a protein-encoding gene, splicing signal for introns, maintenance of the correct reading frame of that gene to permit proper translation of mRNA and stop codons. In some examples, the promoter is a T7 promoter.

In some aspects, the disclosed vectors include one or more additional components, such as ribozyme sequence(s) and/or a transcription terminator sequence. In specific examples of the disclosed vectors, the last nucleotide of the MPV 5′ trailer sequence is followed by three guanosine residues, a hepatitis delta virus ribozyme sequence, and a T7 terminator.

Exemplary nucleic acids can be prepared by cloning techniques. Examples of appropriate cloning and sequencing techniques, and instructions sufficient to direct persons of skill through many cloning exercises are known (see. e.g., Sambrook et al. (Molecular Cloning: A Laboratory Manual, 4^{th} ed, Cold Spring Harbor, New York, 2012) and Ausubel et al. (In Current Protocols in Molecular Biology, John Wiley & Sons, New York, through supplement 104, 2013). Product information from manufacturers of biological reagents and experimental equipment also provide useful information. Such manufacturers include the SIGMA Chemical Company (Saint Louis, MO), R&D Systems (Minneapolis. MN), Pharmacia Amersham (Piscataway, NJ), CLONTECH Laboratories, Inc. (Palo Alto, CA), Chem Genes Corp., Aldrich Chemical Company (Milwaukee, WI), Glen Research, Inc., GIBCO BRL Life Technologies, Inc. (Gaithersburg, MD), Fluka Chemica-Biochemika Analytika (Fluka Chemie AG, Buchs, Switzerland), Invitrogen (Carlsbad, CA), and Applied Biosystems (Foster City, CA), as well as many other commercial sources known to one of skill.

Nucleic acids can also be prepared by amplification methods. Amplification methods include polymerase chain reaction (PCR), the ligase chain reaction (LCR), the transcription-based amplification system (TAS), the self-sustained sequence replication system (3SR). A wide variety of cloning methods, host cells, and in vitro amplification methodologies are well known to persons of skill.

The nucleic acid molecules encoding a disclosed immunogen (e.g., rMPV) can include a recombinant DNA which is incorporated into a vector into an autonomously replicating plasmid or virus or into the genomic DNA of a prokaryote or eukaryote, or which exists as a separate molecule (such as a cDNA) independent of other sequences. The nucleotides can be ribonucleotides, deoxyribonucleotides, or modified forms of either nucleotide. The term includes single and double stranded forms of DNA.

DNA sequences encoding the disclosed immunogen can be expressed in vitro by DNA transfer into a suitable host cell. The cell may be prokaryotic or eukaryotic. The term also includes any progeny of the subject host cell. It is understood that all progeny may not be identical to the parental cell since there may be mutations that occur during replication. Methods of stable transfer, meaning that the foreign DNA is continuously maintained in the host, are known in the art.

Hosts can include microbial, yeast, insect, and mammalian organisms. Methods of expressing DNA sequences having eukaryotic or viral sequences in prokaryotes are well-known. Non-limiting examples of suitable host cells include bacteria, archea, insect, fungi (for example, yeast), plant, and animal cells (for example, mammalian cells, such as human cells). Exemplary cells of use include Escherichia coli, Bacillus subtilis, Saccharomyces cerevisiae, Salmonella typhimurium, SF9 cells, C129 cells, 293 cells, Neurospora, and immortalized mammalian myeloid and lymphoid cell lines. Techniques for the propagation of mammalian cells in culture are well-known (see, e.g., Helgason and Miller (Eds.), 2012, Basic Cell Culture Protocols (Methods in Molecular Biology), 4^{th} Ed., Humana Press). Examples of commonly used mammalian host cell lines include VERO, HeLa, CHO, WI38, BHK, and COS cell lines, although other cell lines may be used, such as cells designed to provide higher expression, desirable glycosylation patterns, or other features. In some aspects, the host cells include BHK cells or derivatives thereof such as BHK BSR T7/5 cells.

Transformation of a host cell with recombinant DNA can be carried out by conventional techniques as are well-known to skilled persons. Where the host is prokaryotic, such as, but not limited to, E. coli, competent cells capable of DNA uptake can be prepared from cells harvested after exponential growth phase and subsequently treated by the CaCl₂ method using well-known procedures. Alternatively, MgCl₂ or RbCl can be used. Transformation can also be performed after forming a protoplast of the host cell if desired, or by electroporation.

When the host is a eukaryote, the cells can be transformed/transfected with the disclosed nucleic acid molecules or vector by any one of a number of known methods, including, for example, calcium phosphate precipitation, mechanical procedures such as microinjection, electroporation, or lipofection. Eukaryotic cells can also be co-transformed with polynucleotide sequences encoding a disclosed immunogen, and a second foreign DNA molecule encoding a selectable phenotype, such as the herpes simplex thymidine kinase gene. Another method is to use a eukaryotic viral vector, such as simian virus 40 (SV40) or bovine papilloma virus, to transiently infect or transform eukaryotic cells and express the protein (see for example, Viral Expression Vectors, Springer press, Muzyczka ed., 2011).

Modifications can be made to a nucleic acid molecule or vector encoding a disclosed rMPV without diminishing its biological activity. Some modifications can be made to facilitate the cloning, expression, or purification of rMPV.

### VII. Methods of Eliciting an Immune Response

The disclosed immunogens (e.g., a rMPV expressing a SARS-CoV-2 spike protein, or an immunogenic composition thereof), can be used in methods of inducing an immune response to SARS-CoV-2 to prevent, inhibit (including inhibiting transmission), and/or treat a SARS-CoV-2 infection.

Provided herein are methods of eliciting an immune response against SARS-CoV-2 in a subject by administering to the subject an effective amount of a rMPV expressing SARS-CoV-2 S protein, or an immunogenic composition thereof, as disclosed herein. In some aspects, the rMPV or immunogenic composition is administered intranasally. Upon immunization, the subject responds by producing antibodies specific for SARS-CoV-2 S protein. In addition, innate and cell-mediated immune responses are induced, which can provide antiviral effects as well as regulation of the immune response. As a result of the immunization, the host becomes at least partially or completely immune to SARS-CoV-2 infection, or resistant to developing moderate or severe SARS-CoV-2 disease (such as COVID-19), particularly of the lower respiratory tract.

A subject who has or is at risk for developing a SARS-CoV-2 infection, for example because of exposure or the possibility of exposure to the SARS-CoV-2, can be selected for immunization. Following administration of a disclosed immunogen, the subject can be monitored for infection or symptoms associated with SARS-CoV-2 infection.

To identify subjects for prophylaxis or treatment according to the methods of the disclosure, accepted screening methods are employed to determine risk factors associated with a targeted or suspected disease or condition, or to determine the status of an existing disease or condition in a subject. These screening methods include, for example, conventional work-ups to determine environmental, familial, occupational, and other such risk factors that may be associated with the targeted or suspected disease or condition, as well as diagnostic methods, such as various ELISA and other immunoassay methods to detect and/or characterize SARS-CoV-2 infection. These and other routine methods allow the clinician to select patients in need of therapy using the methods and immunogenic compositions of the disclosure. In accordance with these methods and principles, a composition can be administered according to the teachings herein, or other conventional methods, as an independent prophylaxis or treatment program, or as a follow-up, adjunct or coordinate treatment regimen to other treatments.

The disclosed immunogens can be used in coordinate (or prime-boost) immunization protocols or combinatorial formulations. In certain aspects, novel combinatorial immunogenic compositions and coordinate immunization protocols employ separate immunogens or formulations, each directed toward eliciting an anti-SARS-CoV-2 immune response, such as an immune response to SARS-CoV-2 spike protein. Separate immunogenic compositions that elicit the anti-SARS-CoV-2 immune response can be combined in a polyvalent immunogenic composition administered to a subject in a single immunization step, or they can be administered separately (in monovalent immunogenic compositions) in a coordinate immunization protocol.

In one aspect, a suitable immunization regimen includes at least two separate inoculations with one or more immunogenic compositions including a disclosed rMPV with a second inoculation being administered more than about two, about three to eight, or about four weeks following the first inoculation. A third inoculation can be administered several months after the second inoculation, and in specific aspects, more than about five months after the first inoculation, more than about six months to about two years after the first inoculation, or about eight months to about one year after the first inoculation. Periodic inoculations beyond the third may be desirable to enhance the subject's immune memory. The adequacy of the vaccination parameters chosen, e.g., formulation, dose, regimen and the like, can be determined by taking aliquots of serum from the subject and assaying antibody titers during the course of the immunization program. Alternatively, the T cell populations can be monitored by conventional methods. In addition, the clinical condition of the subject can be monitored for the desired effect, e.g., prevention of SARS-CoV-2 infection, improvement in disease state (e.g., reduction in viral load), or reduction in transmission frequency. If such monitoring indicates that vaccination is sub-optimal, the subject can be boosted with an additional dose of immunogenic composition, and the vaccination parameters can be modified in a fashion expected to potentiate the immune response. Thus, for example, a dose of a disclosed immunogen can be increased or the route of administration can be changed.

It is contemplated that there can be several boosts, and that each boost can be a different immunogen. It is also contemplated in some examples that the boost may be the same immunogen as another boost, or the prime.

The prime and the boost can be administered as a single dose or multiple doses, for example, two doses, three doses, four doses, five doses, six doses or more can be administered to a subject over days, weeks or months. Multiple boosts can also be given, such one to five, or more. Different dosages can be used in a series of sequential inoculations. For example, a relatively large dose in a primary inoculation and then a boost with relatively smaller doses. The immune response against the selected antigenic surface can be elicited by one or more inoculations of a subject.

Live-attenuated vaccines of pneumoviruses and paramyxoviruses generally have reduced immunogenicity when administered as a boost given shortly (within one or several months) following an initial immunization. Typically, the second dose is highly restricted in replication by immunity induced by the first dose and as a consequence, the second dose induces a reduced or negligible secondary immune response. For example, the measles, mumps and rubella (MMR) vaccine, which includes live-attenuated versions of the measles and mumps paramyxoviruses, usually is given as a first dose at 12-15 months (to avoid neutralization by maternal antibodies) with a second dose given at 4-6 years. The long interval between the two doses allows immunity to drop sufficient to permit take of the second dose. Poor immunogenicity of booster doses also has been documented in clinical trials of experimental live-attenuated vaccines for other paramyxoviruses and pneumoviruses, such as parainfluenza virus type 3 (Karron et at, Pediatr Infect Dis J 22(5):394-405, 2003, Karron et al., Pediatr Infect Dis J 30(10):e186-191, 2011; Englund et al., Vaccine 31(48):5706-5712, 2013), RSV (Wright et al., J Infect Dis 182(5):1331-1342, 2000; Karron et al., J Infect Dis 191(7):1093-1104, 2005), and a chimeric bovine/human PIV3 virus expressing the RSV F protein from a supernumerary gene (Bernstein et al., Pediatr Infect Dis J 31(2):109-114, 2012) that is analogous to MPV expressing RSV F protein or SARS-CoV-2 F protein. In contrast to the above-listed studies, it is demonstrated herein that administration of a second dose of rMPV resulted in strong increases in the level of local and systemic immunity, both humeral and cellular. This was an unexpected but highly desirable feature of the disclosed live-attenuated rMPV.

The ability to boost immunity is desirable for any vaccine, but it is particularly important for vaccines against respiratory viruses. This is because immune responses in the respiratory tract are less effective in restricting virus infection and replication than systemic immune responses. The present disclosure provides a means for inducing immunity both systemically and in the respiratory tract with a single dose. It also provides a means for boosting a prior immunization. In the present disclosure, this boost was accomplished following a homologous primary immunization, and was shown to boost both systemic and local respiratory tract immunity. However, it is expected that a boost immunization with the rMPV disclosed herein will also be effective following a heterologous primary immunization, such as by a protein subunit or mRNA vaccine. In these cases of parenteral primary immunizations, the rMPV vector would not only boost systemic immunity, but also would induce local respiratory tract immunity that otherwise would not be efficiently induced by a parenteral vaccine.

In several aspects, a disclosed immunogen can be administered to the subject simultaneously with the administration of an adjuvant. In other aspects, the immunogen can be administered to the subject after the administration of an adjuvant and within a sufficient amount of time to elicit the immune response. In other aspects, no adjuvant is administered.

SARS-CoV-2 infection does not need to be completely inhibited for the methods to be effective. For example, elicitation of an immune response to SARS-CoV-2 can reduce or inhibit SARS-CoV-2 infection by a desired amount, for example, by at least 10%, at least 20%, at least 30%, at least 40%, at least 50%, at least 60%, at least 70%, at least 80%, at least 90%, at least 95%, at least 98%, or even at least 100% (elimination or prevention of detectable SARS-CoV-2 infected cells), as compared to SARS-CoV-2 infection in the absence of immunization. In additional examples, SARS-CoV-2 replication can be reduced or inhibited by the disclosed methods. SARS-CoV-2 replication does not need to be completely eliminated for the method to be effective. For example, the immune response elicited using one or more of the disclosed immunogens can reduce SARS-CoV-2 replication by a desired amount, for example, by at least 10%, at least 20%, at least 30%, at least 40%, at least 50%, at least 60%, at least 70%, at least 80%, at least 90%, at least 95%, at least 98%, or even at least 100% (elimination or prevention of detectable SARS-CoV-2 replication), as compared to SARS-CoV-2 replication in the absence of the immune response. In other examples, illness caused by SARS-CoV-2 need not be completely inhibited or eliminated for the methods to be effective. For example, severe lower respiratory disease can be reduced by at least 10%, at least 20%, at least 30%, at least 40%, at least 50%, at least 60%, at least 70%, at least 80%, at least 90%, at least 95%, at least 98%, or even at least 100% (elimination or prevention of detectable SARS-CoV-2 lower respiratory disease).

Following immunization of a subject, serum can be collected from the subject at appropriate time points, frozen, and stored for neutralization testing. Methods to assay for neutralization activity, include, but are not limited to, plaque reduction neutralization (PRNT) assays, microneutralization assays, flow cytometry-based assays, single-cycle infection assays, and pseudovirus neutralization assays.

### EXAMPLES

The following examples are provided to illustrate particular features of certain aspects of the disclosure, but the scope of the claims should not be limited to those features exemplified.

### Example 1: MPV Vectors Expressing SARS-CoV-2 S Protein

This example describes the construction of rMPV vectors encoding WT or modified forms of the SARS-CoV-2 S protein for evaluation as live-attenuated vaccine candidates for SARS-CoV-2. As described herein, wild-type MPV (strain 15) was modified to express various versions of WT and modified SARS-CoV-2 S protein from a heterologous gene that was modified with putative MPV transcription signals to be expressed by the MPV polymerase into a separate mRNA.

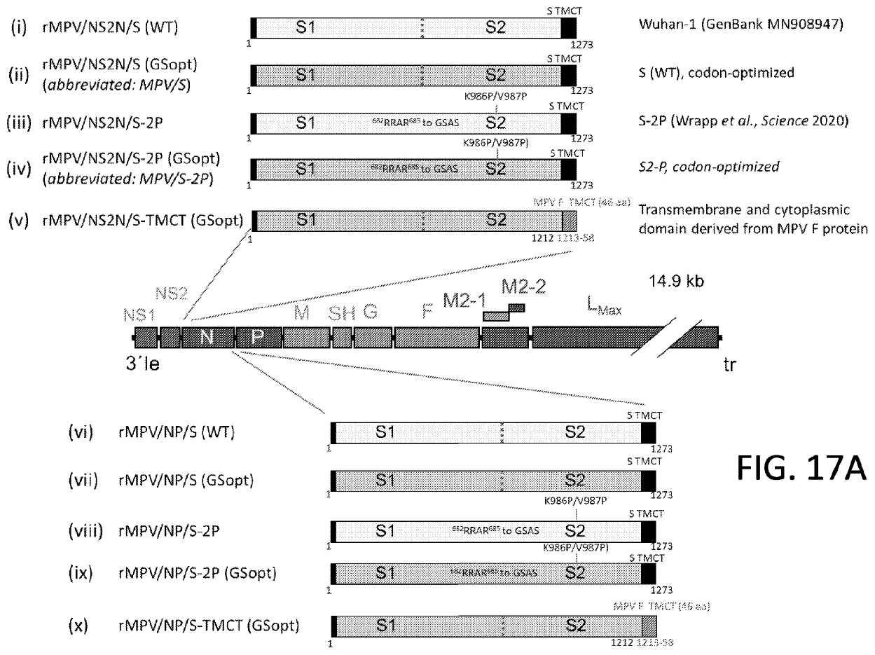

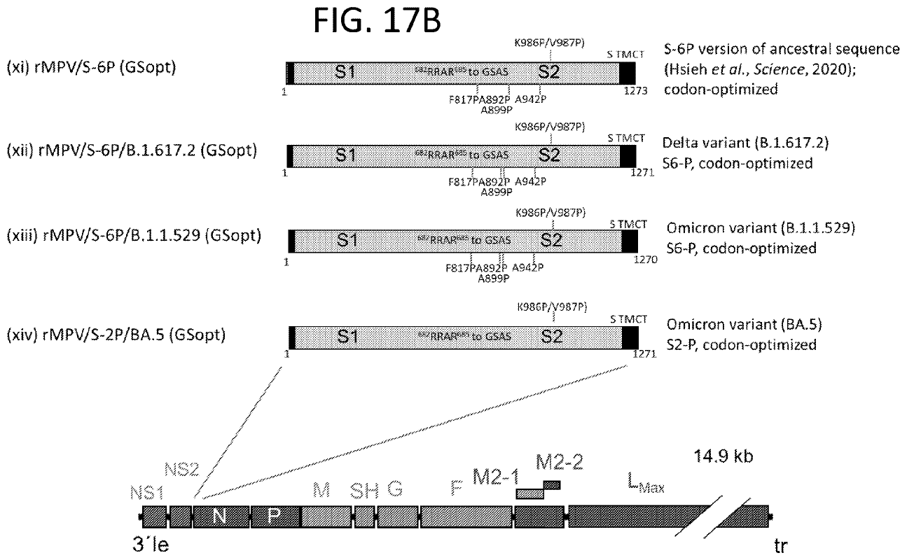

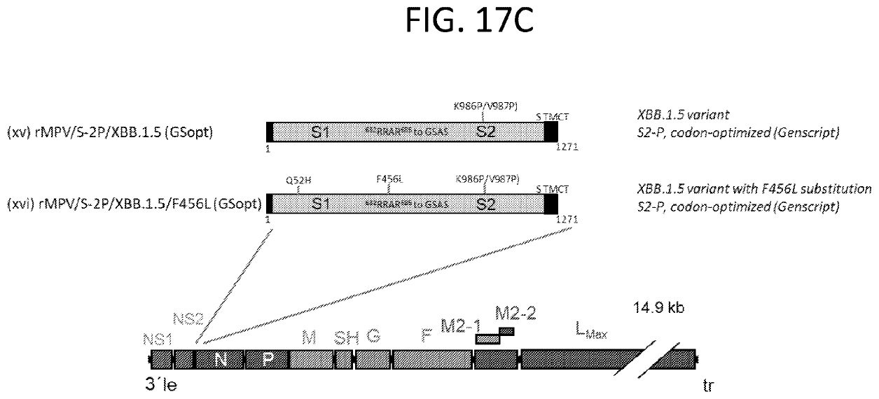

cDNA sequences encoding different versions of the SARS-CoV-2 S protein based on the ancestral Wuhan-1 strain (GenBank Accession No. MN908947; SEQ ID NO: 33) were designed and modified for expression from MPV strain 15 (FIGS. 17A-17B). In each construct, the S ORF is flanked by putative MPV gene start and gene end transcription signals to enable transcription of the S gene as a separate mRNA. The nucleotide sequence of the S ORF (GENBANK accession number MN908947) was scanned for sequences similar to the MPV gene end signal consensus sequence. Two potential sites with a high degree of similarity were identified in the S ORF: AGTTAGATAAATA (SEQ ID NO: 46), at nucleotides 3452-3464 of the S ORF; and AGTATATAAAAT (SEQ ID NO: 47), at nucleotides 3623-3634 of the S ORF. To avoid premature transcription termination by the MPV polymerase complex due to the presence of these two regions in the added MPV gene, nucleotide changes that did not alter the S protein amino acid sequence were introduced into these regions. Specifically, nucleotides 3452-3464 of the S ORF, encoding amino acids E1151, L1152, D1153, and K1154, were changed to AGTTGGATAAGTA (SEQ ID NO: 48); and nucleotides 3623-3624 of the S ORF, encoding Q1208, Y1209, 11210, and K1211, were changed to AGTACATCAAGT (SEQ ID NO: 49). The ancestral sequence of the Wuhan-1 S ORF with these silent changes is referred to herein as wildtype (WT) S.

The rMPV constructs disclosed herein are summarized in Table 1.

TABLE 1
Recombinant MPV constructs expressing SARS-CoV-2 S protein

| | | SEQ ID |
|---|---|---|
| Construct name | Features | NO: |
| rMPV/NS2N/S (WT) | WT (Wuhan-1 strain) S ORF inserted between the MPV | 5 |
| | NS2 and N genes | |
| rMPV/NS2N/S (GSopt) | Codon-optimized WT S ORF inserted between the MPV | 7 |
| (“MPV/S”) | NS2 and N genes | |
| rMPV/NS2N/S-2P | S ORF encoding S protein with R682G, R683S, R685S, | 9 |
| | K986P, and V987P substitutions, inserted between the | |
| | MPV NS2 and N genes | |
| rMPV/NS2N/S-2P (GSopt) | Codon-optimized S ORF encoding S protein with K986P, | 11 |
| (“MPV/S-2P”) | V987P, R682G, R683S and R685S substitutions, inserted | |
| | between the MPV NS2 and N genes | |
| rMPV/NS2N/S-TMCT (GSopt) | Codon-optimized S ORF encoding an S protein in which | 13 |
| | the transmembrane/cytoplasmic tail (TMCT) is replaced by | |
| | the TMCT of the MPV F protein, inserted between the | |
| | MPV NS2 and N genes | |
| rMPV/NP/S (WT) | WT (Wuhan-1 strain) S ORF inserted between the MPV N | 15 |
| | and P genes | |
| rMPV/NP/S (GSopt) | Codon-optimized WT (Wuhan-1 strain) S ORF inserted | 17 |
| | between the MPV N and P genes | |
| rMPV/NP/S-2P | S ORF encoding S protein with R682G, R683S, R685S, | 19 |
| | K986P, and V987P substitutions, inserted between the | |
| | MPV N and P genes | |
| rMPV/NP/S-2P (GSopt) | Codon-optimized S ORF encoding S protein with K986P, | 21 |
| | V987P, R682G, R683S and R685S substitutions, inserted | |
| | between the MPV N and P genes | |
| rMPV/NP/S-TMCT (GSopt) | Codon-optimized S ORF encoding an S protein in which | 23 |
| | the TMCT is replaced by the TMCT of the MPV F protein, | |
| | inserted between the MPV N and P genes | |
| rMPV/S-6P (GSopt) | Codon-optimized S ORF encoding an S protein with | 31 |
| | R682G, R683S, R685S, F817P, A892P, A899P, A942P, | |
| | K986P, and V987P substitutions, inserted between the | |
| | MPV NS2 and N genes | |
| rMPV/S-6P/B.1.617.2 (GSopt) | Codon-optimized S ORF encoding Delta variant | 25 |
| | (B.1.617.2) S protein with R682G, R683S, R685S, F817P, | |
| | A892P, A899P, A942P, K986P, and V987P substitutions, | |
| | inserted between the MPV NS2 and N genes | |
| rMPV/S-6P/B.1.1.529 (GSopt) | Codon-optimized S ORF encoding Omicron variant | 27 |
| | (B.1.1.529) S protein with R682G, R683S, R685S, F817P, | |
| | A892P, A899P, A942P, K986P, and V987P substitutions, | |
| | inserted between the MPV NS2 and N genes | |
| rMPV/S-2P/BA.5 (GSopt) | Codon-optimized S ORF encoding Omicron variant (BA.5) | 29 |
| | S protein with R682G, R683S, R685S, K986P, and V987P | |
| | substitutions, inserted between the MPV NS2 and N genes | |
| rMPV/S-2P/XBB.1.5 (GSopt) | Codon-optimized S ORF encoding XBB.1.5 variant S | 50 |
| | protein with R682G, R683S, R685S, K986P, and V987P | |
| | substitutions, inserted between the MPV NS2 and N genes | |
| rMPV/S-2P/XBB.1.5/F456L | Codon-optimized S ORF encoding XBB.1.5 variant S | 52 |
| (GSopt) | protein including the Q52H and F456L XBB.1.5 | |
| | substitutions, with R682G, R683S, R685S, K986P, and | |
| | V987P substitutions, inserted between the MPV NS2 and | |
| | N genes | |

Recombinant MPV having an S ORF with the “2P” (K986P and V987P) or “6P” (F817P, A892P, A899P, A942P, K986P, and V987P) substitutions express S protein stabilized in the prefusion conformation. The R682G, R683S, R685S substitutions eliminate a furin cleavage site between the S1 and S2 subunits, which blocks the cleavage of S into the S1 and S2 subunits and prevent fusion activity by the S protein. The S ORFs in which the transmembrane and cytoplasmic tail (TMCT) regions of the S protein were replaced with MPV F gene sequences were generated to increase incorporation of the S protein into the MPV vector particle. Antigen packaged in a virus particle has the potential to be more immunogenic than antigen released from necrotic cells due to several factors, including faster release from infected cells, more efficient uptake by antigen-presenting cells due both to the particle and to attachment, increased antigen stability, and presentation in an ordered concentrated array.

The DNA fragments were synthesized commercially and cloned into a full-length cDNA of the MPV antigenome contained in a plasmid. For virus recoveries from cDNA, full-length cDNA plasmids containing the antigenome sequences of the viruses shown in FIGS. 17A-17B were transfected into BHK BSR T7/5 cells, together with N, P, L, and M2-1 support plasmids. On the following day, BHK BSR T7/5 cells were scraped into the media and transferred onto sub-confluent Vero cells. Cells were co-cultured for 12 days or until cytopathic effect (CPE) typical for MPV was visible. Virus stocks were harvested by scraping the monolayers into the overlying cell culture medium. Supernatants and cells were vortexed to detach virus particles from cells. Supernatants were clarified by centrifugation prior to aliquoting. Vials were snap-frozen on dry ice (passage 1; P1). Aliquots were tittered by plaque assay on Vero cells, and fresh monolayers of Vero cells were infected at a multiplicity of infection (MOI) of 0.01. After harvesting and titration of virus stocks, supernatants of P2 were used to amplify these viruses one more time, generating P3 stocks. P3 stocks were tittered by dual-staining immunoplaque assay, and the titers and fractions of S-expressing viruses were calculated (Table 2).

All of the recombinant viruses were recovered, and complete genome sequencing showed that they were free of adventitious mutations evident by sequence analysis of uncloned RT-PCR products. There was a general trend for higher titers of stocks of rMPV expressing S antigens from the third promoter proximal position compared to expression from the fourth genome position (Table 2, compare “NS2N” and “NP” versions). This trend persisted for stocks of P1 through P3, in particular for MPV/NS2N/S-2P (GSopt) vs. MPV/NP/S-2P (GSopt). Thus, insertion of the S antigens in the third genome position was better tolerated by MPV.

The first passages of MPV/NS2N and MPV/NP constructs encoding the TMCT version of the S protein in the third genome position yielded low titers; in case of insertion in the fourth genome position, recombinant viruses with TMCT versions had small-plaque phenotypes. Following titration of the P3 stock of MPV/NS2N/S-TMCT, immunoplaque staining showed that only 13% of virus plaques were positive for S antigen, revealing substantial loss of S-TMCT expression. Thus, the replacement of the TMCT region of S by that of the MPV F protein seemed to negatively affect MPV replication and stability of expression of the S antigen when expressed from the third genome position. In contrast, expression from the fourth genome position was much more stable, with 89% of plaques expressing SARS-CoV-2 S.

The set of viruses with insertion of S in the 3^{rd} and 4^{th} genome position included WT and GSopt versions of the S-2P ORF, allowing for comparison of the effect of S-2P stabilization on virus replication. Unexpectedly, for MPV/NS2N and MPV/NP viruses, P1 stocks of the S-2P stabilized versions had 8- to 30-fold lower titers than corresponding versions encoding the non-stabilized S proteins. Comparison of pairs of rMPV vectors which differed only by codon optimization or not of the S ORFs—whether WT S or S-2P,—showed that codon optimization appeared to have negligible effects on MPV replication. Because of potential positive effects on immunogenicity of the S protein, codon optimization for human expression was desirable. Indeed, by P3, titers above 6.0 Log₁₀ PFU per mL could reliably be obtained for MPV/NS2N/S-2P (GSopt). Immunoplaque assays showed that it was feasible to generate virus stocks with S expression in >95% of MPV plaques. Stocks with lower levels of S expression were also identified, showing that it is important to control for stability of S expression by this highly sensitive dual-staining immunoplaque assay.

TABLE 2
Titers of MPV vectors expressing versions
of the SARS-C-V-2 S Protein^{a}

| | | | | P3: Fraction |
|---|---|---|---|---|
| Virus Name | Titer (Log₁₀ PFU/mL)^{b} | | | with S |
| Replica number | P1 | P2 | P3 | expression |
| MPV/NS2N/S (WT)^{c} | | | | |
| 1 | 4.9 | 6.9 | nd^{e} | nd |
| 2 | 4.9 | 7.0 | nd | nd |
| MPV/NS2N/S (GSopt)^{d} | | | | |
| 1 | 5.0 | 6.6 | nd | nd |
| 2 | 5.1 | nd | nd | nd |
| MPV/NS2N/S-2P | | | | |
| 1 | 4.1 | 6.7 | 7.1 | 44 |
| 2 | 4.2 | 6.8 | 7.1 | 93 |
| MPV/NS2N/S-2P (GSopt) | | | | |
| 1 | 3.9 | 6.9 | 6.7 | 75 |
| 2 | 3.9 | 6.8 | 6.9 | 97 |
| MPV/NS2N/S-TMCT (GSopt) | | | | |
| 1 | 2.7 | 6.8 | 7.2 | 13 |
| MPV/NP/S (WT) | | | | |
| 1 | 4.9 | 6.6 | nd | Nd |
| 2 | 4.9 | 6.7 | nd | Nd |
| MPV/NP/S (GSopt) | | | | |
| 1 | 4.0 | 6.4 | nd | nd |
| 2 | 4.5 | 6.4 | nd | nd |
| MPV/NP/S-2P | | | | |
| 1 | 4.1 | 6.2 | 6.8 | 93 |
| 2 | 4.4 | 6.9 | 6.9 | 99 |
| MPV/NP/S-2P (GSopt) | | | | |
| 1 | 3.6 | 5.6 | 6.2 | 93 |
| 2 | 3.4 | 5.4 | 6.3 | 100 |
| MPV/NP/S-TMCT (GSopt) | | | | |
| 1 | 3.8^{f} | 6.0^{f} | 6.1^{f} | 93 |

^{a}For virus recoveries from cDNA, full-length cDNA plasmids containing the antigenome sequences of the indicated viruses were transfected into BHK BSR T7/5, together with N, P, L, and M2-1 support plasmids. On the following day, BHK BSR T7/5 were scraped into the media and transferred onto subconfluent Vero cells. Cells were co-cultured for 12 days or until cytopathic effect (CPE) typical for MPV was visible. Virus stocks were harvested by scaping of the monolayers into the supernatant. Supernatants and cells were vortexed to detach virus particles from cells, and supernatants were clarified by centrifugation prior to aliquoting. Vials were snap-frozen on dry ice [passage 1 (P1)]. Aliquots were tittered by plaque assay on Vero cells, and fresh monolayers of Vero cells were infected at a multiplicity of infection (MOI) of 0.01. After harvesting and titration of virus stocks, supernatants of P2 were amplified one more time to generate P3. P3 was tittered by dual-staining immunoplaque assay, and the titer and the fraction of S expressing viruses was calculated.
^{b}Passage number (P).
^{c}WT; the sequence of the SARS-CoV-2 S ORF corresponds to that of the ancestral SARS-CoV-2 isolate Wuhan-1.
^{d}GSopt; codon-optimized for human codon usage (Genscript, Inc).
^{e}nd, not done.
^{f}Small-plaque phenotype.

### Example 2: Materials and Methods

This example describes the materials and experimental procedures for Examples 3-10.

| | | |
|---|---|---|
| REAGENT or RESOURCE | SOURCE | IDENTIFIER |

Antibodies

| | | |
|---|---|---|
| Anti-human IgD-FITC | Southern Biotech | Cat #2030-02; RRID: |
| | | AB_2795624 |
| Anti-human CD3-BB700, Clone SP34-2 | BD Biosciences | Cat #566517; RRID: |
| | | AB_2744378 |
| Anti-human CD16-BB700, Clone 3G8 | BD Biosciences | Cat #746199; RRID: |
| | | AB_2743545 |
| Anti-human CD14-BB700, Clone M5E2 | BD Biosciences | Cat #745790; RRID: |
| | | AB_2743246 |
| Anti-human IgM-BV510, Clone G20-127 | BD Biosciences | Cat #563113; RRID: |
| | | AB_2738010 |
| Anti-human CD27-BV711, Clone O323 | Biolegend | Cat #302834; RRID: |
| | | AB_2563809 |
| Anti-human CD20-BV785, Clone 2H7 | Biolegend | Cat #302356; RRID: |
| | | AB_2566316 |
| Anti-human CD21-BUV395, Clone B-ly4 | BD Biosciences | Cat #740288; RRID: |
| | | AB_2740027 |
| Anti-human IgG-BUV737, Clone G18-145 | BD Biosciences | Cat #612819; RRID: |
| | | AB_2738986 |
| Anti-human CD138-APC, Clone MI15 | Biolegend | Cat #356506; RRID: |
| | | AB_2561880 |
| Anti-human CD38-PE, Clone OKT10 | Caprico Biotechnologies | Cat #100826 |
| Anti-human IgA-TXRD | Southern Biotech | Cat #2050-07; RRID: |
| | | AB_2795705 |
| Anti-human CD19-PE/Cy5, Clone J3-119 | Beckman | Cat #IM2643U |
| Anti-human CD95-PE/Cy7, Clone DX2 | Biolegend | Cat #305622; RRID: |
| | | AB_2100369 |
| Anti-human CD69-FITC, Clone FN50 | Biolegend | Cat #310903; RRID: |
| | | AB_314838 |
| Anti-human Granzyme B-BV421, Clone | BD Biosciences | Cat #563389; |
| GB11 | | RRID: AB_2738175 |
| Anti-human CD8a-eFluor 506, Clone RPA-T8 | Thermo Fisher | Cat #69-0088-42; |
| | | RRID: AB_2637468 |
| Anti-human IL-2-BV605, Clone MQ1-17H12 | Biolegend | Cat #500332; |
| | | RRID: AB_2563877 |
| Anti-human IFNg-BV711, Clone 4S.B3 | Biolegend | Cat #502540; |
| | | RRID: AB_2563506 |
| Anti-human IL-17A-BV785, Clone BL168 | Biolegend | Cat D#512338; RRID: |
| | | AB_2566765 |
| Anti-human TNFa-BUV395, Clone Mab11 | BD Biosciences | Cat #563996; |
| | | RRID: AB_2738533 |
| Anti-human CD4-BUV496, Clone SK3 | BD Biosciences | Cat #612937 |
| Anti-human CD95-BUV737, Clone DX2 | BD Biosciences | Cat #612790 |
| Anti-human CD3-BUV805, Clone SP34-2 | BD Biosciences | Cat #742053; |
| | | RRID: AB_2871342 |
| Anti-human CD107a-AF647, Clone H4A3 | Biolegend | Cat #328612; |
| | | RRID: AB_1227506 |
| Anti-human CD107b-AF647, Clone H4B4 | Biolegend | Cat #354312; |
| | | RRID: AB_2721405 |
| Anti-human CD103-PE, Clone B-Ly7 | eBioscience | Cat #12-1038-42; |
| | | RRID: AB_11150242 |
| Anti-human CD28-PE/Dazzle 594, Clone | Biolegend | Cat #302942; |
| CD28.2 | | RRID: AB_2564235 |
| Anti-human Ki-67-PE-Cy7, Clone B56 | BD Biosciences | Cat #561283; |
| | | RRID: AB_10716060 |
| Anti-human Foxp3-AF700, Clone PCH101 | Thermo Fisher | Cat #56-4776-41; |
| | | RRID: AB_1582210 |
| Anti-monkey IgG(H + L)-HRP | Thermo Fisher | Cat #PA1-84631; |
| | | RRID: AB_933605 |
| Anti-monkey IgA(alpha chain)-biotin | Alpha Diagnostic | Cat #70049 |
| | International | |
| Rabbit hyperimmune serum against MPV | Brock et al., J Virol | N/A |
| virions | 86: 5829-5843, 2012 | |
| Rabbit hyperimmune serum against MVA | Krempl et al., J Virol | N/A |
| expressing MPV G | 81: 9490-9501, 2007 | |
| Goat hyperimmune serum against SARS- | Liu et al., Proc Natl | N/A |
| CoV-2 S-2P | Acad Sci USA | |
| | 118(50): e2109744118, | |
| | 2021 | |
| Anti-rabbit IRDye 680RD IgG | Li-Cor | Cat #926-68073; |
| | | RRID: AB_10954442 |
| Anti-goat IRDye 800CW IgG | Li-Cor | Cat #926-32214; |
| | | RRID: AB_621846 |
| Anti-human IRDye 800CW IgG | Li-Cor | Cat #926-32232; RRID: |
| | | AB_10806644 |
| Anti-rabbit Alexa Fluor 647 IgG (H+L) | ThermoFisher | Cat #A21245 |

Virus Strains

| | | |
|---|---|---|
| SARS-CoV-2 USA-WA1/2020 isolate | CDC | GenBank MN985325; |
| | | GISAID: EPI_ISL_404895 |
| SARS-CoV-2 lineage B.1.1.7/Alpha | CDC | USA/CA_CDC_5574/2020 |
| | | isolate |
| | | GISAID: EPI_ISL_751801 |
| SARS-CoV-2 lineage B.1.351/Beta | Dr. A Pekosz, Johns | USA/MD-HP01542/2021 |
| | Hopkins University | isolate GISAID: |
| | | EPI_ISL_890360 |

Chemicals, Peptides, and Recombinant Proteins

| | | |
|---|---|---|
| SARS-CoV-2 S-2P | Liu et al., Proc Natl Acad | |
| | Sci USA | |
| | 118(50): e2109744118, | |
| | 2021 | |
| SARS-CoV-2 S RBD | Liu et al., Proc Natl Acad | |
| | Sci USA | |
| | 118(50): e2109744118, | |
| | 2021 | |
| SARS-CoV-2 S-2P-Avi | Described herein | Avi-tagged S-2P |
| SARS-CoV-2 S RBD-Avi-His | Described herein | Avi-tagged RBD |
| Viability Dye eFluor780 | Thermo Fisher | Cat #65-0865-14 |
| Live/Dead fixable blue dead cell stain | Thermo Fisher | Cat #L23105 |
| kit, for UV excitation | | |
| BV421 Streptavidin | Biolegend | Cat #405226 |
| BV605 Streptavidin | BD Biosciences | Cat #563260 |
| Ficoll-Paque density gradient | Cytiva | Cat #17144003 |
| Monensin solution | ThermoFisher | Cat #00-4505-51 |
| Brefeldin A solution | ThermoFisher | Cat #00-4506-51 |
| Peptivator SARS-CoV-2 Prot_S1 | Miltenyi Biotec | Cat #130-127-048 |
| Peptivator SARS-CoV-2 Prot_S+ | Miltenyi Biotec | Cat #130-127-312 |
| Peptivator SARS-CoV-2 Prot_S | Miltenyi Biotec | Cat #130-127-953 |
| Peptivator SARS-CoV-2 Prot_N | Miltenyi Biotec | Cat #130-126-699 |
| cBioscience intracellular fixation & | Thermo Fisher | Cat #88-8824-00 |
| permeabilization buffer set | | |
| eBioscience permeabilization buffer | Thermo Fisher | Cat #00-83333-56 |
| Buffer AVL | Qiagen | Cat #19073 |
| Trizol LS Reagent | Thermo Fisher | Cat #10296028 |

Critical Commercial Assays

| | | |
|---|---|---|
| DELFIA time resolved fluorescence | Perkin Elmer | N/A |
| immunoassay | | |
| V-PLEX SARS-CoV-2 Panel 25 (ACE2) | MSD | Cat #K15586U |
| V-PLEX SARS-CoV-2 Panel 27 (ACE2) | MSD | Cat #K15609U |
| V-PLEX SARS-CoV-2 Panel 32 (ACE2) | MSD | Cat #K15671U |
| Amicon centrifugal filters | Millipore | Cat #UFC500396 |
| NHP XL Cytokine Luminex Premixed kit | R&D systems | Cat #FCSTM21 |
| QuickChange Lightning Multi Site-directed | Agilent | Cat #210514 |
| mutagenesis kit | | |
| QIAamp Viral RNA Mini Kit | Qiagen | Cat #52904 |
| PureLink RNA Mini Kit | Thermo Fisher | Cat #12183018A |
| TaqMan RNA-to-Ct 1-Step Kit | Thermo Fisher | Cat #4392938 |
| Pierce Silver Stain Kit | Thermo Fisher | Cat #24612 |

Experimental Models: Cell lines

| | | |
|---|---|---|
| BSR T7/5 | Buchholz et al., J Virol | |
| | 73: 251-259, 1999 | |
| A549 | ATCC | Cat #CCL-185 |
| Vero | ATCC | Cat #CCL-81 |
| Vero E6 | ATCC | Cat #CRL-1586 |
| Vero E6 expressing human TMPRSS2 | Liu et al., Proc Natl | N/A |
| | Acad Sci USA | |
| | 118(50): c2109744118, | |
| | 2021 | |

Experimental Models: Organisms/strains

| | | |
|---|---|---|
| Rhesus macaques (Macaca mulatta) | Described herein | Young adult animals, |
| | | 4-7 years of age, male. |
| | | Animals tudy protocol |
| | | approved by NIAID ACUC |
| Human plasma from SARS-CoV-2 | NIAID, NIH | Exempt from IRB review |
| convalescent donors, de-identified | | |

Oligonucleotides

| | | |
|---|---|---|
| 5′-CGATCTCTTGTAGATCTGTTCTC-3′ | SEQ ID NO: 40 | Subgenomic E Forward |
| 5′-ATATTGCAGCAGTACGCACACA-3′ | SEQ ID NO: 41 | Subgenomic E Reverse |
| 5′- | SEQ ID NO: 42 | Subgenomic E Probe |
| ACACTAGCCATCCTTACTGCGCTTCG-3′ | | |
| 5′-GACCCCAAAATCAGCGAAAT-3′ | SEQ ID NO: 43 | Genomic N Forward |
| 5′-TCTGGTTACTGCCAGTTGAATCTG-3′ | SEQ ID NO: 44 | Genomic N Reverse |
| 5′-ACCCCGCATTACGTTTGGTGGACC-3′ | SEQ ID NO: 45 | Genomic N Probe |

Recombinant DNA

| | | |
|---|---|---|
| MPV | Krempl et al., J Virol | N/A |
| | 81: 9490-9501, 2007 | |
| MPV/S | Described herein | N/A |
| MPV/S-2P | Described herein | N/A |
| MPV N, P, M2-1, and L helper plasmids | Krempl et al., J Virol | N/A |
| | 81: 9490-9501, 2007 | |
| SARS-CoV-2 S | Liu et al., Proc Natl | GenBank MN908947 |
| | Acad Sci USA | |
| | 118(50): e2109744118, | |
| | 2021 | |
| SARS-CoV-2 S-2P | Wrapp et al., Science | N/A |
| | 367: 1260-1263, 2020 | |
| SARS-CoV-2 S-6P | Gencrated as described in | N/A |
| | Hsieh et al., Science | |
| | 369: 1501-1505, 2020 | |
| SARS-CoV-2 S RBD | Wrapp et al., Science | N/A |
| | 367: 1260-1263, 2020 | |
| SARS-CoV-2 S-2P-Avi | Zhou et al., Cell Rep | N/A |
| | 33: 108322, 2020 | |
| SARS-CoV-2 S RBD-Avi | Described herein | N/A |

Software

| | | |
|---|---|---|
| FlowJo v10 | BD | flowjo.com |
| Prism v9 | GraphPad Software, LLC | graphpad.com/ |
| | | scientificsoftware/ |
| | | prism/ |
| ImageStudioLite 5.2.5 | Li-Cor | licor.com/bio/image- |
| | | studio-lite/ |
| | | resources#is15 |
| Relative Quantification, Standard Curve | Thermo Fisher | apps.thermofisher.com |
| BioTek Gen 5 | BioTek | biotek.com/products/ |
| | | software-robotics- |
| | | software/gen5- |
| | | microplate- |
| | | reader-and-imager- |
| | | software/ |

### Cell Lines

Baby hamster kidney (BHK) cells expressing T7 RNA polymerase (BSR T7/5) were used to recover MPV and derivatives, and were grown in Glasgow minimum essential medium (MEM) (Thermo Fisher Scientific) supplemented with 10% fetal bovine serum (FBS), 2 mM L-glutamine (Thermo Fisher Scientific), and 2% MEM Amino Acids (Thermo Fisher Scientific). African green monkey kidney Vero (ATCC CCL-81) and Vero E6 (ATCC CRL-1586) cells were grown in OptiMEM (Thermo Fisher) supplemented with 5% FBS and 0.1 mg/ml penicillin/streptomycin. Human lung epithelial A549 cells (ATCC CCL-185) were grown in F12-K medium (ATCC) supplemented with 10% FBS and 0.1 mg/ml penicillin/streptomycin. Vero and A549 cells were used for the characterization of the MPV vaccine candidates. Vero E6 cells were used for SARS-CoV-2 neutralization assays and titrations, whereas SARS-CoV-2 virus stocks were expanded using Vero E6 cells that express high levels of ACE2 (Chu et al., Lancet Microbe 1:e14-e23, 2020; Ren et al., J Gen Virol 87:1691-1695, 2006) or Vero E6 cells that stably express human TMPRSS2 (Liu et al., Proc Natl Acad Sci USA 118(50):e2109744118, 2021).

### SARS-CoV-2 Virus Stocks

The SARS-CoV-2 USA-WA1/2020 challenge virus (lineage A; GenBank MN985325 and GISAID accession ID: EPI_ISL_404895) was passaged twice on Vero E6 cells. The USA/CA_CDC_5574/2020 isolate (lineage B.1.1.7, GISAID: EPI_-ISL_751801) and the USA/MD-HP01542/2021 isolate (lineage B.1.351, GISAID: EPI_-ISL_890360) were passaged on Vero E6 cells stably expressing TMPRSS2. Titration of SARS-CoV-2 stocks was performed by determination of the TCID₅₀ in Vero E6 cells. Whole-genome Illumina deep sequencing analysis confirmed that the complete genome sequences of the SARS-CoV-2 challenge virus pool and the B.1.1.7 and B.1.351 variants were identical to that of consensus sequences, except for minor backgrounds of reads (<10%). All experiments with SARS-CoV-2 were conducted in Biosafety Level-3 containment laboratories.

### Generation of MPV and Derivatives

The rMPV vector backbone used in this study was derived from the MPV strain 15 cDNA cloned in a pBluescript vector under control of a T7 promoter (Brock el al., J Viral 86:5829-5843, 2012). The downstream 67% of the L ORF were codon pair optimized for efficient expression in humans. The MPV/S and MPV/S-2P vectors were constructed using a previously-described MPV reverse genetics system (Krempl et al., J Virol 81:9490-9501, 2007). For MPV/S, the full-length 1,273 amino acid (aa) S protein ORF of Wuhan-Hu-1 (GenBank MN908947) was codon optimized for human expression (Genscript) and synthesized commercially (BioBasic). To generate MPV/S-2P, two proline substitutions (aa 986 and 987) were introduced to stabilize S in the prefusion conformation, and four an mutations (RRAR to GSAS, aa 682-685 of SEQ ID NO: 33 and SEQ ID NO: 34, respectively) were inserted to ablate the furin cleavage site between S1 and S2 (Wrapp et al., Science 367:1260-1263, 2020). S and S-2P inserts were designed so that the ORF was flanked by MPV gene start and gene end transcription signals to enable transcription of S, and the Kozak consensus sequence GCCGCCACC was placed immediately upstream of the S AUG start codon to provide efficient context for translation initiation as previously described (Brock et al., J Virol 86:5829-5843, 2012). S and S-2P genes were inserted at the third gene position between the MPV NS2 and N genes using XmaI and KpnI restriction sites.

MPV and derivatives were recovered from cDNA as previously described (Brock et alt, J Virol 86:5829-5843, 2012). All in vitro tissue culture experiments were done at 32° C. unless otherwise noted. In brief, BHK BSR-T7/5 cells that constitutively express the T7 RNA polymerase were transfected using Lipofectamine 3000 with the MPV antigenome plasmid and support plasmids expressing MPV N, P, M2-1, and L proteins. After an overnight incubation at 37° C., the cells were scraped into the media to generate a cell suspension that was cocultured with a sub-confluent Vero cell monolayer for approximately 2 weeks to generate passage 1 (P1) viral stocks. Virus stocks of MPV and MPV/S were propagated on Vero cells using an MOI≥0.01 PFU to generate working virus stocks. Viruses were harvested about 2 weeks post-infection, when CPE typical for MPV infection disrupted the monolayer. Viruses were further passaged once (MPV) or twice (MPV/S-2P) on Vero cells using an MOI≥0.01 PFU to generate working stocks (P2 of MPV; P3 of MPV/S-2P). Viral RNA was isolated from working stocks using the QIAamp Viral RNA kit (Qiagen), and the genomic sequences were confirmed by Sanger sequencing of overlapping RT-PCR amplicons that covered the whole viral genome with the exception of the regions complementary to the genome-end specific primers (covering 24 and 27 nucleotides of the leader and trailer ends, respectively).

### Dual-Staining Immunoplaque Assay

Virus titers were determined by dual-staining immunoplaque assay on Vero cells under a 0.8% methylcellulose overlay. In brief, len-fold serial dilutions were incubated in duplicate wells on Vero cells under methylcellulose overlay. After a 5- to 6-day incubation at 32° C., monolayers were fixed using 80% methanol, MPV was detected using a rabbit hyperimmune MPV antiscrum, and SARS CoV-2 S and S-2P proteins were detected using the human monoclonal antibody CR3022. Anti-rabbit IRDye680 and anti-human IRDye800 infrared fluorophore-labeled secondary antibodies were used to visualize MPV and S, pseudocolored in red and green, respectively, with double-positive plaques appearing yellow/green.

### Multicycle Kinetics of MPV and Derivatives in Cell Culture

Sub-confluent monolayers of Vero or A549 cells in 12-well plates were infected in duplicate with an MOI of 0.1 PFU/cell of MPV, MPV/S, or MPV/S-2P and incubated at 32° C. for 10 days. Vero-grown virus was harvested daily for 10 days, and A549-grown virus was harvested on days 1, 2, 3, 6, 8, and 10 pi. At time of harvest, cells were scraped into the media, vortexed three times for 10 seconds each, and centrifuged for 5 minutes at 850×g. Clarified supernatants were aliquoted and snap-frozen in dry ice prior to storage at −80° C. The virus titer of each sample was determined by dual-staining immunoplaque assay.

### SDS-Page and Western Blotting

Sub-confluent monolayers of Vero or A549 cells in 12-well plates were infected with an MOI of 2 PFU/cell of MPV, MPV/S, or MPV/S-2P and incubated at 32° C. At 56 hpi, supernatants were removed, and cells were gently washed with 1×PBS before being lysed with 200 ml SDS-PAGE lysis buffer containing protease inhibitors. Cell lysates were clarified using a Qiashredder column (Qiagen) following the manufacturer recommendations and snap-frozen at −80° C. until use. NuPAGE Sample Reducing Agent (Thermo Fisher) was added to cell lysates, and proteins were denatured and reduced at 90° C. for 10 minutes prior to loading onto 4-15% TGX gels (BioRad). After electrophoresis, proteins were transferred to polyvinylidene difluoride (PVDF) membranes that were next blocked with LiCor blocking buffer for 1 hour at room temperature (RT). Then, membranes were incubated at 4° C. overnight with 1:25,000 rabbit polyclonal MPV hyperimmune antisera, 1:10,000 mouse polyclonal tubulin antibody (Abeam) and 1:5,000 goat hyperimmune antiserum N25-154, raised against recombinantly-expressed amino acids 1-1208 of the SARS-CoV-2 S-2P protein (Liu et al., Proc Natl Acad Sci USA 118(50):e2109744118, 2021). After washing five times with PBS supplemented with 0.1% Tween 20, membranes were incubated with infrared-dye labelled secondary antibodies (1:10,000 of donkey anti-rabbit IgG IRDye 680, donkey anti-goat IgG IRDye 800, and donkey anti-mouse IRDye 800, LiCor) at RT for 45 minutes. After incubation, membranes were washed five times with PBS supplemented with 0.1% Tween 20, followed by three washes with PBS before imaging on the Odyssey CLX. Protein bands were quantified using the Image Studio software (LiCor) and normalized to tubulin. Lysates were collected and analyzed in two independent experiments. In each experiment, two wells of a 12-well plate were lysed and analyzed separately.

### Sucrose Purification of MPV Virus Stock and Derivatives

Aliquots of Vero cell-grown viruses were purified by ultracentrifugation using 30/60% discontinuous sucrose gradients centrifuged at 134,000×g for 2 hours at 4° C. Virus particles collected at the 30/60% sucrose interface were diluted in OptiMEM media to decrease the sucrose concentration, snap frozen and stored at −80° C. Prior to analysis, viruses were thawed and centrifuged at 20,000×g for 2 hours to pellet the virus. Media were removed and virus pellets were resuspended in Tris-EDTA-NaCl (TEN) buffer for measurement of protein content. Protein concentrations were determined using a BCA assay (Pierce Rapid Gold BCA Protein Assay Kit, Thermo Fisher) prior to SDS-PAGE and Western blotting or silver staining (Pierce Silver Stain Kit, Thermo Fisher) using 1 μg of protein per well.

### Electron Microscopy of Sucrose-Purified MPV Preparations

For electron microscopy analysis of purified virus, two ml of Vero grown MPV and MPV/S-2P were incubated with 1:100 goat anti-S hyperimmune serum for 40 minutes at RT with rotation. After incubation, the virus-antibody mixture was transferred to a 30/60% sucrose gradient in 5 mL thin-wall ultra-clear tubes (Beckman) and centrifugated at 160,000×g at 4° C. for 2 hours. Purified virus bands at the 30/60% interface were collected and fixed with a final concentration of 2% paraformaldehyde in PBS. Immunogold staining to detect SARS-CoV-2 S was carried out as previously described (Park et at, NPJ Vaccines 7:72, 2022). Briefly, 10 μl of purified virus with bound primary antibodies were adsorbed for 30 minutes to freshly glow-discharged 200 mesh Formvar/carbon-coated Ni grids in saturated humid chambers at RT. Then, grids were washed with PBS, blocked with 2% bovine serum albumin (BSA) in PBS followed by 0.1% acetylated BSA (BSA-c, Aurion) in PBS. All samples were labeled with a donkey anti-goat secondary antibody conjugated to 6 nm gold beads (Aurion, cat #25803) previously diluted 1:50 in 0.1% BSA-c in PBS according to the manufacturer's instructions. Labeled grids were sequentially washed with 0.1% BSA-c in PBS, PBS, and then in water followed by negative-staining with methylamine vanadate and electron microscopy observation.

### Immunization and Challenge of Rhesus Macaques

Two separate animal experiments were conducted, each with twelve juvenile to young adult male Indian-origin rhesus macaques (Macaca mulatta). All macaques were confirmed to be seronegative for SARS-CoV-2 prior to immunization intranasally (0.5 ml per nostril) and intratracheally (1 ml) with a total dose of 6.3 log₁₀ PFU of MPV empty vector, MPV/S, or MPV/S-2P. Study #1 compared the replication and immunogenicity of a single dose of MPV, MPV/S, and MPV/S-2P while study #2 compared the replication, immunogenicity, and protective efficacy of a single dose of MPV or MPV/S-2P with a prime-boost regimen of MPV/S-2P. Animals were monitored daily from day −3 through the end of the study. Each time macaques were sedated, they were checked for weight, rectal temperature, heart rate, respiratory rate, and blood oxygen levels.

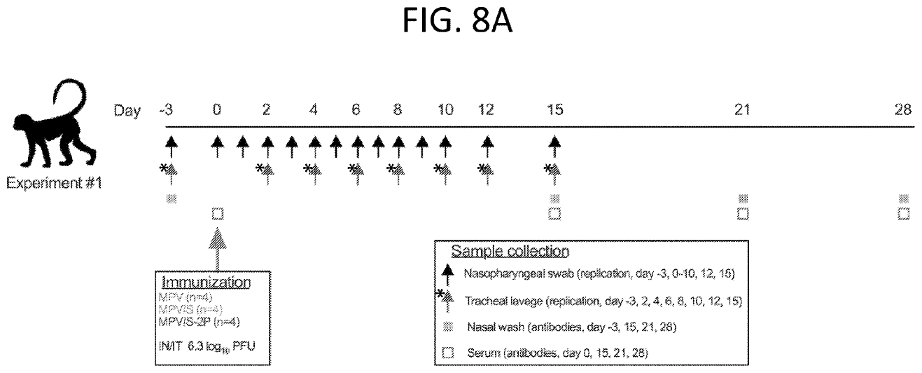

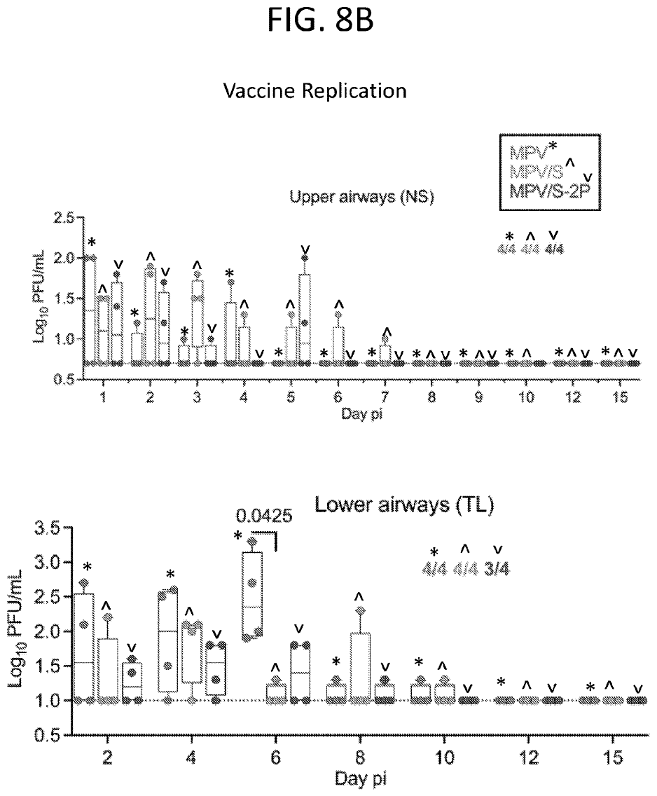

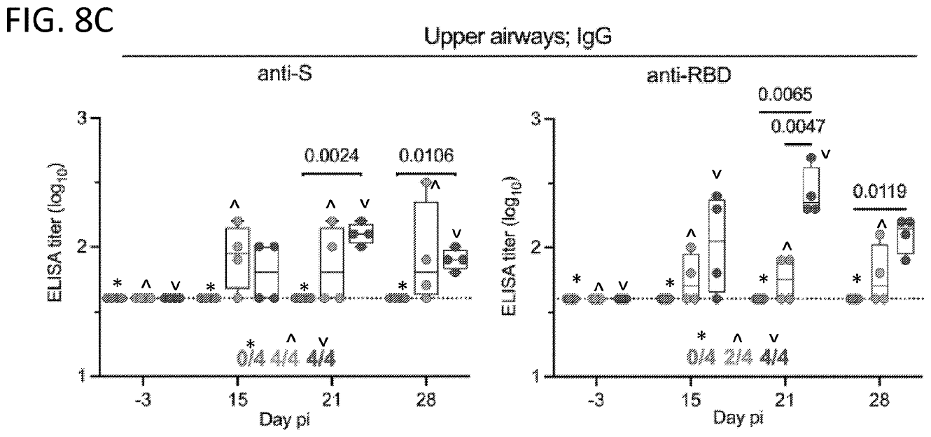

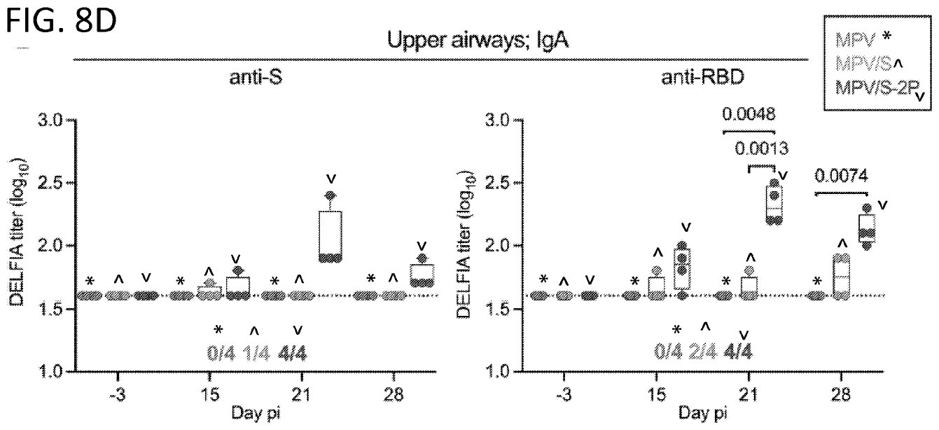

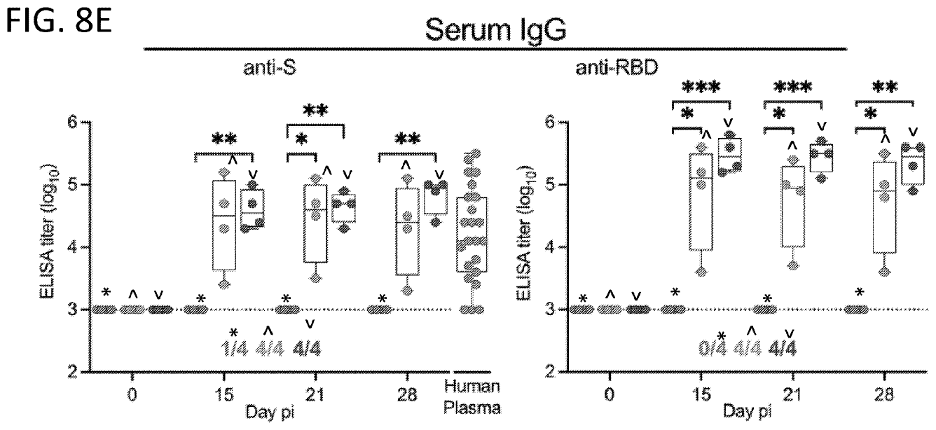

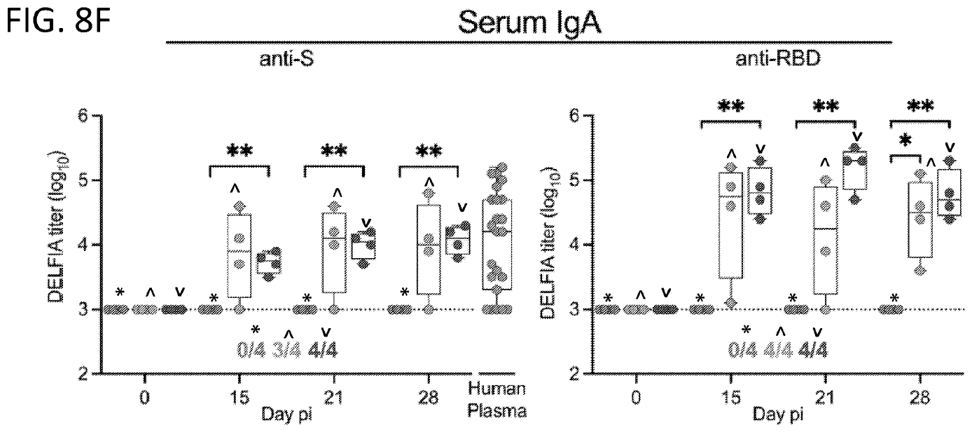

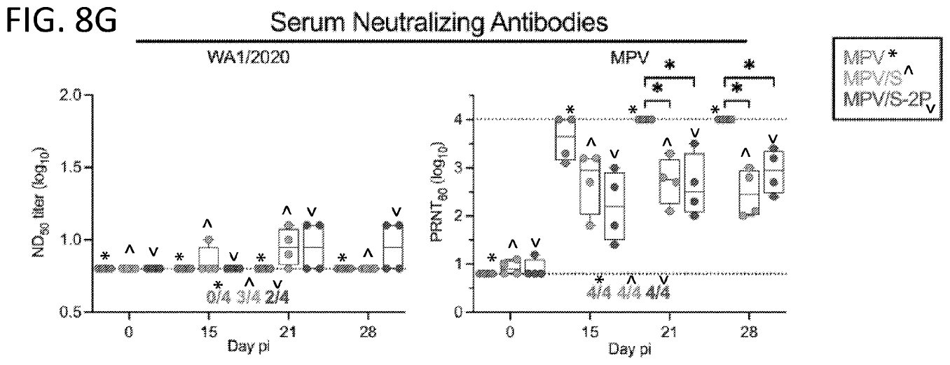

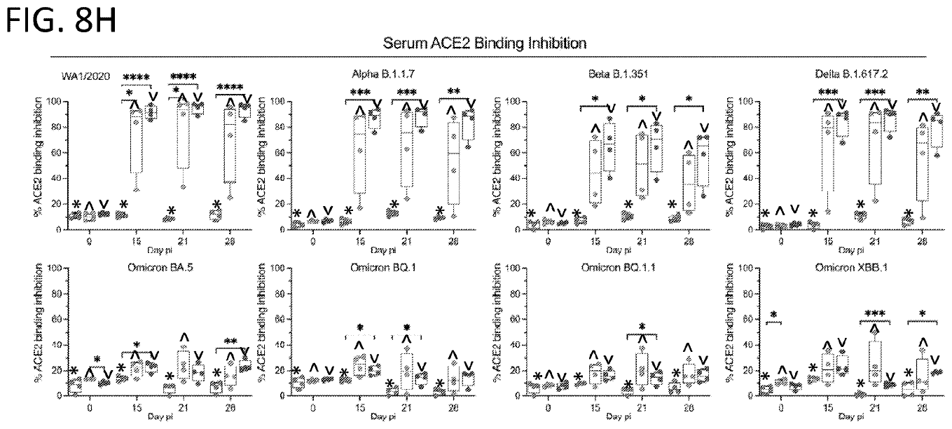

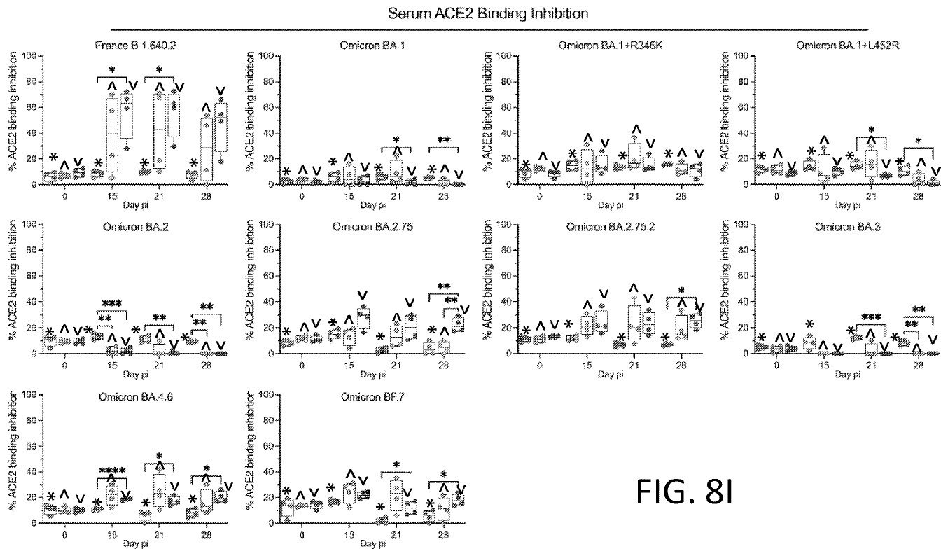

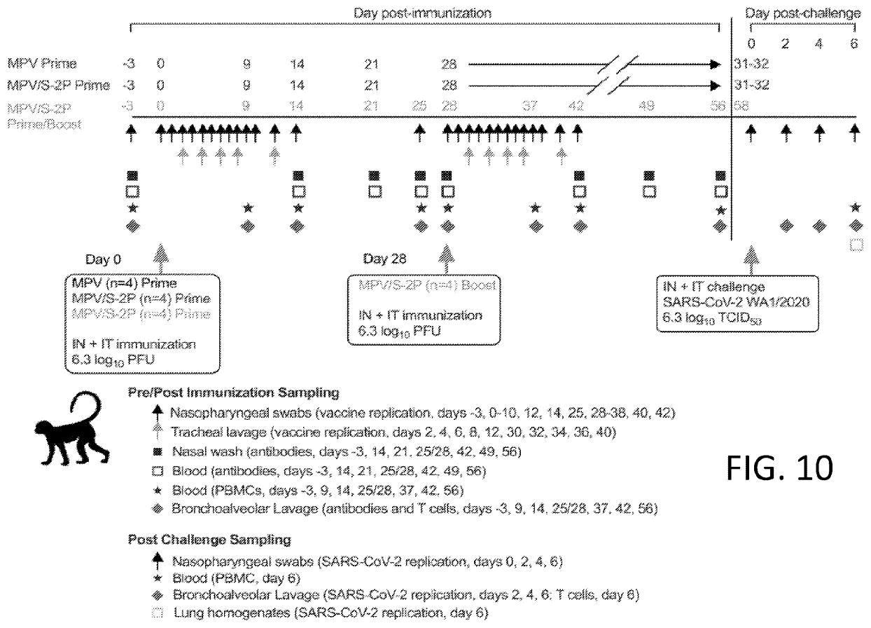

The timelines and sampling schedules for the two separate macaque experiments are diagrammed in FIG. 8A (study #1) and FIG. 10 (study #2). In both studies, nasopharyngeal swabs (NS) were performed on days −3, 0 to 10, 12, and 14 or 15 to evaluate vaccine virus replication in the UA. In the second study, NS were also collected from macaques on day 25 (which is 3 days before the boost) and on days 0 to 10, 12, and 14 or 15 following the MPV/S-2P boost, corresponding to days 28-38, 40 and 42. NS were collected using cotton-tipped applicators, placed in 2 ml Leibovitz (L-15) medium with 1× sucrose phosphate (SP) used as a stabilizer and vortexed for 10 seconds. Then aliquots were snap frozen in dry ice and stored at −80° C. Tracheal lavages (TL) were collected after each dose on days 2, 4, 6, 8, 10 (study #1 only), 12, 15 (study #1 only) to quantify vaccine virus replication in the LA. TLs were mixed 1:1 with L-15 medium containing 2×SP and aliquots were snap frozen on dry ice and stored at −80° C. Nasal washes (NW) for analysis of mucosal antibodies in the UA were performed on days −3, 14 or 15, 21, and 25 or 28, for primed macaques and on days 42, 49 and 56 for MPV/S-2P boosted macaques using 1 ml of Lactated Ringer's solution per nostril. NW samples were aliquoted, snap frozen on dry ice, and stored at −80° C. Blood was collected on days 0, 15, 21, and 28 pi in study #1 for analysis of serum antibodies. In study #2, blood was collected on days −3, 9, 14, 21, 25 or 28 after the prime dose. In addition, blood was collected on days 37, 42, 49 and 56 from the macaques that received the MPV/S-2P boost dose. Blood collected in study #2 was used for analysis of serum antibodies and/or peripheral blood mononuclear cells (PBMCs). Bronchoalveolar lavages (BAL) were performed only in study #2 and were performed using 3 times 10 ml of 1×PBS on days −3, 9, 14, and 25 or 28 after the prime dose. In addition, BAL was collected on days 37, 42 and 56 from the macaques that received the MPV/S-2P boost dose. BAL was used for analysis of mucosal antibodies as well as analysis of mononuclear cells in the LA. For isolation of cells, BAL was filtered through a 100 μm filter and centrifuged at 544×g for 15 minutes at 10° C. The cell pellets were resuspended at concentrations of 2×10⁷ cells/ml in X-VIVO 15 media with 10% FBS. The BAL supernatant was aliquoted, snap frozen on dry ice, and stored at −80° C. (FIG. 10).

In study #2, four weeks after immunization (or four weeks after boost for animals that received a prime/boost regimen), macaques were transferred to a BSL3 laboratory four weeks after immunization (or four weeks after boost for animals that received a prime/boost regimen). On day 31-32 for primed macaques (two animals per group per day for technical reasons) or day 58 for primed/boosted macaques, animals were challenged intranasally and intratracheally with 10^{6.3} log₁₀ TCID₅₀ of SARS-CoV-2 USA-WA1/2020. Post-challenge sample collections were performed using the same methodology described for the post-immunization sampling. NS were performed on days 0, 2, 4, and 6 post-challenge (pe), blood was collected on day 6 pc for collection of PBMC, and BAL was collected on days 2, 4, and 6 pc. Mononuclear cells from BAL were only collected on day 6 pc. On day 6 pc, animals were euthanized and six separate samples from individual lung lobes were collected, snap frozen in dry ice, and stored at −80° C.

### Replication of MPV and Derivatives in the Upper and Lower Airways of Macaques

MPV, MPV/S, and MPV/S-2P shedding in the upper and lower airways of macaques was evaluated by dual-staining immunoplaque assay of NS and TL samples collected at the indicated time post-immunization (pi). Briefly, ten-fold serial dilutions were incubated in duplicate wells on Vero cells under methyl-cellulose overlay. After an 11-day incubation at 32° C., monolayers were fixed using 80% methanol, MPV was detected using a rabbit hyperimmune MPV antiserum, and SARS CoV-2 S and S-2P proteins were detected using the human monoclonal antibody CR3022. Anti-rabbit IRDyc680 and anti-human IRDye800 infrared fluorophore-labeled secondary antibodies were used to visualize MPV and S, pseudocolored in red and green, respectively, with double-positive plaques appearing yellow/green.

### Dual Anti-S and Anti-RBD IgG and IgA ELISA

Levels of serum and mucosal anti-SARS-CoV-2 S or its receptor binding domain (RBD) IgG and IgA antibodies elicited by MPV/S and MPV/S-2P were determined using a dual IgG/IgA or IgG/dimeric IgA ELISA protocol developed by modification of both ELISA and dissociation-enhanced lanthanide fluorescent (DELFIA) time resolved fluorescence (TRF) immunoassay.

Serum, BAL, and NW samples were heated at 56° C. for 30 minutes to inactivate complement and reduce potential risk from any residual viruses. The treated samples were immediately used or stored at −80° C. for later use. Black ninety-six-well plates (MaxiSorp, Thermo Fisher Scientific, cat #437111) were coated with 100 μl/well of SARS-CoV-2 S-2P or S-6P (1 μg/ml) or RBD (2 μg/ml) (Hsieh et al., Science 369:1501-1505, 2020; Liu et al., Proc Natl Acad Sci USA 118(50):e2109744118, 2021) in 50 mM carbonate coating buffer (generated by mixing 3.7 g sodium bicarbonate with 0.64 g sodium carbonate (Sigma) in 1 L molecular biology grade water). After overnight incubation at 4° C., plates were washed three times with washing buffer (PBS (Gibco, cat #14190-144) containing 0.1% IGEPAL CA-630) using a BioTek 405 micro-plate washer and blocked with 250 μl of blocking buffer (1×DPBS containing 5% dry milk (W/V), Nestle, Carnation nonfat dry milk). After overnight incubation at 4° C., plates were washed one additional time with washing buffer.

Serum or mucosal samples were serially diluted in 96-well plates, starting dilution at 1:1000 for serum and 1:100 for BAL and NW samples, followed by ten 3-fold serial dilutions using sample dilution buffer (PBS+5% dry milk+0.2% IGEPAL CA-630). Then, 100 μl of diluted samples were transferred from the dilution plates to the antigen-coated assay plates in duplicate. After 1-hour incubation at RT on a rotating shaker, plates were washed 3 times with 250 μl washing buffer, and incubated on a rotating shaker for another 20 minutes. 100 μl per well of secondary goat anti-monkey IgG(H+L)-HRP (ThermoFisher, cat #PA1-84631) at 1:10,000 dilution or goat anti-monkey IgA-alpha chain specific Biotin (Alpha Diagnostic International, cat #70049) at 1:5,000 or mouse anti-rhesus J chain-biotin (NHPRR, cat #PR-3316) at 1:5,000 to detect dimeric IgA, diluted in dilution buffer, was added, and plates were incubated for 1 hour. After incubation, plates were washed three times and 100 μl per well of diluted Streptavidin-Europium (PerkinElmer, cat #1244-360) diluted at 1:2,000 in PBS+0.2% IGEPAL CA-630 was added. Plates were incubated for 1 hour and then washed three times. Then, 50 μl of Pierce ECL (ThermoFisher, cat #32106) per well was added and after 10 minutes incubation, plates were read on the Synergy neo (BioTek) plate reader and luminescence data for the IgG assay were collected. After collecting the first set of data, plates were washed three times, and 100 μl per well of pre-warmed RT enhancement solution (PerkinElmer: 4001-0010) was added. Plates were further incubated for 20 minutes while rocking. After incubation, plates were read again on the Synergy neo (BioTek) plate reader using a program for TRF (using the excitation 360/40 and emission 620/40 parameters) and luminescence data for the IgA assay were collected.

Finally, data were processed as follows: (i) the average reading for each sample from duplicate wells was calculated; (ii) the average reading from the blank samples was subtracted from the average reading for each sample; and (iii) the cut off value was set to the blank average plus three standard deviations of blank readings. Then, the IgG and IgA titers of each sample were determined by interpolating the sigmoid standard curve generated on Prism 9.0.

### Serum Antibody Avidity Assay

The avidity of serum antibodies to S-6P was determined by modification of the dual IgG and IgA ELISA protocol described above. After incubation of the diluted serum samples with S-6P and before the addition of the secondary antibody mixture, one set of plates was incubated with 100 μl of PBS while the other set of duplicate plates were treated with 100 μl of 1.0 M sodium thiocyanate solution (NaSCN; Sigma-Aldrich) for 15 minutes at RT to strip S-6P from low affinity binding antibodies. Then, plates were washed three times, and secondary antibody mixture was added. Anti-S IgG and IgA titers of each serum sample in the presence or absence of NaSCN were calculated. The avidity index (AI) was calculated using the ratio of the NaSCN-treated IgG or IgA titers to the PBS-treated serum IgG or IgA titers.

### Serum Neutralization Assay of SARS-CoV-2 and MPV

Neutralization assays to determine serum antibody titers against SARS-CoV-2 WA1/2020, B.1.1.7, or B.1.351 of immunized macaques were done in a BSL3 laboratory. Sera were heat inactivated at 56° C. for 30 minutes and two-fold serially diluted in Opti-MEM prior to incubation 1:1 with 100 TCID₅₀ of SARS-CoV-2 for 1 hour at 37° C. Mixtures were added to quadruplicate wells of Vero E6 cells in 96-well plates and incubated for four days. The 50% neutralizing dose (ND₅₀) was the highest dilution of serum that prevented cytopathic effect in 50% of the wells. Serum neutralizing antibody titers against the MPV vector were also quantified on Vero cells using 60% plaque reduction neutralization tests (PRNT₆₀).

### ACE2 Binding Inhibition Assays

As a complement to the neutralization assay, the ability of heat inactivated serum and BAL fluid from immunized macaques to inhibit the binding of ACE2 to soluble SARS-CoV-2 spikes (Meso Scale Diagnostics, cat #K15586U, K156091J, and K15671U) was evaluated. Serum samples were diluted 1:20 and BAL and NW samples were diluted 1:2 using the diluent supplied with the kit. Each sample was evaluated in duplicate, and plates were prepared and analyzed as previously described (Le Nouen et al., Cell 185:4811-4825, 2022). Briefly, plates in which each well was coated with 10 different soluble spike proteins were blocked for 1 hour using MSD blocker A buffer followed by a wash with MSD washing buffer. Then, diluted test samples were added, and plates were further incubated for 2 hours on a plate shaker at RT. Sulfo-Tag labelled soluble ACE2 was then added and after a 1-hour incubation, plates were washed. The MSD GOLD electrochemiluminescence read buffer B was next added, and chemiluminescence of bound ACE2-Sulfo-Tag was acquired on a Meso 1300 Quickplex reader. The average electrochemiluminescence signals in duplicate wells for each sample were determined, as well as the maximum electrochemiluminescence signals of ACE2/S protein binding in no-sample control wells. The ACE2 binding inhibition by each test sample is shown as percent inhibition relative to no-sample controls.

### Antibody-Dependent Cellular Phagocytosis (ADCP)

To investigate stimulation of phagocytic activity induced by anti-S antibodies in serum samples of immunized macaques, ADCP assays were performed using a modification of a previously published method (Ackerman et al., J Immunol Methods 366:8-19, 2011).

The SARS-CoV-2 S-2P protein (corresponding to the sequence of the ancestral Wuhan-1 strain) was mixed with EZ Link NHS-Biotin (ThermoFischer, cat #20217; 1 mg of biotin powder dissolved in 295 μl of DMSO) for 2 hours on ice. The S-2P protein was mixed with EZ Link NHS-Biotin (ThermoFischer, cat #20217; 1 mg of biotin powder dissolved in 295 μl of DMSO) for 2 hours on ice. Excess biotin was removed by adding 15 ml of PBS to the mixture followed by centrifugation at 1,700×g for 5 minutes at 4° C. using an Amicon 30K MW cut-off membrane (Millipore, cat #UFC903024). This procedure was repeated twice. The last centrifugation was performed until the volume of S-2P protein was between 500 to 700 μl. Then, the purified biotinylated S-2P protein was collected and its concentration was determined by Bradford protein assay.

Forty μl of FluoSpheres Neutravidin-Labelled Microspheres with yellow-green fluorescence (ThermoFisher, cat #F8776) were transferred into a 1.5 ml sterile Eppendorf tube and washed with 1 ml PBS+0.5% BSA. Then, microspheres were centrifugated at 6,800×g for 5 minutes at 4° C. and the supernatant was discarded. Microspheres were washed one more time following the same procedure for a total of two washes. After the second wash, beads were resuspended in 200 μl of PBS+0.5% BSA. Then, 40 μg of purified biotinylated S-2P protein were mixed with the microspheres and the volume was brought to 500 μl using PBS+0.5% BSA. The bead-S2-P mixture was incubated overnight at 4° C. in the dark with rotation. On the following day, conjugated microspheres were washed twice using PBS and once with PBS+0.5% BSA and centrifugated at 6,800×g for 5 minutes at 4° C. between each wash. After the third wash, the conjugated microspheres were resuspended in 400 μl of PBS+0.5% BSA and were stored at 4° C. in the dark for up to one month.

Serum samples from immunized macaques were added to U-bottom 96-well plates (Costar, cat #3799) at a starting dilution of 1:10 and then were three-fold serially diluted in PBS+0.5% BSA for a total of 12 dilutions. One μl of the stock of S-conjugated beads was added to each well of the serum dilutions and plates were incubated for two hours at 37° C. Next, 2×10⁴ THP-1 cells were added to each well and plates were incubated at 37° C. for another two hours. After incubation, plates were washed twice with PBS and centrifugated at 150×g for 5 minutes at RT. After washing, cells were fixed with 2% PFA final volume in PBS+0.5% BSA. Plates were kept at 4° C. in the dark until analysis of FITC signal by flow cytometry on the BD Canto II. The frequency of FITC positive THP-1 cells for each dilution series was used to calculate IC₅₀ values. Serum from one of the MPV/S-2P immunized macaques on day 21 post-boost was included on each assay plate as a positive control sample, and the reported EC₅₀ values were normalized to the average of the positive control values from all analyzed plates.

### Expression and Purification of S and RBD Probes for Identification of S-Specific B Cells

The S probe was expressed from a previously described cDNA that encodes for the prefusion stabilized single-chain Fc tagged SARS-CoV-2 S protein (S-2P, derived from the ancestral Wuhan-1 sequence), conjugated to an Avi tag (Zhou et al., Nature 579:270-273, 2020). This construct was transfected into Expi293 cells and on day 6 post-transfection, the supernatant was harvested, and cells and debris were removed by centrifugation, followed by filtration through a 0.2 μm PES membrane. Then, the supernatant was incubated with Protein A Fast Flow resin (Cytiva) for 2 hours at 4° C. with rotation. The cell supernatant/resin mixture was poured onto gravity flow columns (BioRad, cat #7372512) and resin was washed three times with PBS. To remove the Fe tag and biotinylate S-2P simultaneously, the protein-bound resin was incubated overnight at 4° C. with 200 μg HRV3C (Antoniou et al., Mol Biotechnol 59:407-424, 2017) and biotinylated using a mixture of 10 μg of BirA enzyme and 100 μM biotin (BirA500 kit, Avidity). After incubation, the protein flowthrough that contains the biotinylated S-2P was collected and further purified by size exclusion chromatography in PBS using a Superose 6 Increase column (Cytiva). The purified, biotinylated S-2P protein was concentrated to 1 mg/ml by centrifugation using an Amicon 50 kDa molecular weight cut-off membrane (Millipore) and aliquots were snap-frozen in liquid nitrogen and stored at −80° C. until use.

The RBD cDNA was expressed from the pcDNA3.1(−) vector. The construct encodes from the 5′-end to the 3′-end for (i) the Kozak sequence, (ii) a signal peptide sequence, (iii) aa 328-531 (204 aa) of SARS-CoV-2 S corresponding to the receptor binding domain (RBD), (iv) the 10LnQQ-AVI sequence⁴⁶ and (v) an 8×His tag. The RBD cDNA was cloned into the pCDNA3.1(−) vector between the XbaI and BamHI sites to generate the pcDNA3.1-SARS2_RBD_Avi_His vector that was transfected into Expi293 cells following the manufacturer's instructions. On day 6 post-transfection, cells and cell debris were removed by centrifugation and the supernatant that contained the RBD protein was further clarified through a 0.2 μm PES vacuum filter. The clarified supernatant was then loaded on a HisTrap FF column (Cytiva). The loaded column was washed using a 20 mM HEPES+250 mM NaCl washing buffer and the RBD protein was eluted using the washing buffer supplemented with 300 mM imidazole. After elution, the RBD protein was diluted to ~3 mg/ml in washing buffer and aliquots were snap frozen in liquid nitrogen and stored at −80° C. until further use. Next, aliquots of the RBD protein were biotinylated using the BirA500 kit (Avidity) following the manufacturer's instructions and the biotinylated-RBD was further purified from the excess of biotin by size exclusion chromatography using a Superdex 200-Increase 10/300 GL column in PBS. Aliquots of biotinylated RBD were snap frozen in liquid nitrogen and stored at −80° C. until further use.

### Biolayer Interferometry

The biotinylation and antigenicity of the S-2P and RBD probes were confirmed by biolayer interferometry using the Octet RED384 (ForteBio). Assays were performed at 30° C. in black 96-well plates (Greneir Bio-One #655209) with shaking. The running buffer used was 10× Kinetics Buffer (ForteBio #18-1105) diluted to 1× with PBS. Streptavidin biosensors (ForteBio) were hydrated in 1× running buffer for at least 10 minutes prior to beginning the assays. Human anti-S-specific antibodies 2M-10B11 (Chi et al., Science 369:650-655, 2020) and C105 (Barnes et al., Nature 588:682-687, 2020) were used to confirm binding to the RBD probe, and 0304-3H3 (Chi et al., Science 369:650-655, 2020) and C105 were used to confirm binding to the S-2P probe. Antibodies were serially diluted using seven three-fold dilutions from a starting concentration of 100 μg/ml. Streptavidin biosensors were loaded with biotinylated S-2P (1 μg/ml) or RBD (0.4 μg/ml) for 5 minutes. Biosensors were next washed for 3 minutes in 1× running buffer, and then dipped into antibody dilutions for 5 minutes. After antibody binding, biosensors were moved to 1× running buffer for 20 minutes to monitor dissociation. The background binding observed from a probe-loaded sensor dipped into a control well with no antibody was subtracted from the test wells. The biotinylated S-2P probe efficiently bound to C105 (RBD-specific) and 0304-3H3 (binds outside RBD), while the biotinylated RBD probe efficiently bound to C105 and 2M-10B11 (RBD specific).

Probe conjugation

Streptavidin (SA)-BV605 (BD Biosciences) and streptavidin (SA)-BV421 (BioLegend) were used to label biotinylated S-2P and RBD, respectively, and were made fresh the day of the experiment. The S-2P probe was mixed to SA-BV605 at a 4:1 molar ratio in 1/3 increments, each with a 20-minute incubation, prior to using the conjugated probe for flow cytometry staining. The RBD probe was conjugated to SA-BV421 using the same strategy as described for S-2P. Due to the small molecular weight of the RBD (25,000 Da) the MW of only the SA portion (60,000 Da) of the fluorophore was used to determine the molar ratio. Therefore, the RBD:SA 4:1 molar ratio was used to determine the final quantity of SA-BV421 that was mixed with the RBD following the same protocol than for S-2P. Each conjugated probe was tittered using PBMCs isolated from the blood of a healthy donor from the NIH blood bank. The optimal amount of S-2P and RBD probes was determined to be 0.3 μg of each probe per reaction.

### Identification of S-Specific B Cells in the Blood of Immunized Macaques

To characterize S-specific B cells in the blood of immunized macaques, single-cell suspensions of PBMCs were plated in 96-well plates at ≤3.5×10⁶ cells/ml in 200 μl PBS+1% FBS. Cells were centrifuged at 544×g for 5 minutes at 4° C. and stained first with 0.3 μg each of purified SARS-CoV-2 S-2P and RBD probes, tagged with BV605 and RBD-BV421, respectively, for 30 minutes at RT. To further characterized the S-specific B cells, PBMCs were next washed once with Wash Buffer (PBS+1% FBS+0.05% sodium azide) and then stained with a 50 μl cocktail of fluorochrome-labeled antibodies diluted in Wash Buffer for 20 minutes at 4° C. The antibodies used for surface staining were: IgD (FITC, Southern Biotech), CD3 (BB700, clone SP34-2, BD Biosciences), CD16 (BB700, clone 3G8, BD Biosciences). CD14 (BB700, clone M5E2, BD Biosciences), IgM (BV510, clone G20-427, BD Biosciences), CD27 (BV711, clone 0323, Biolegend), CD20 (BV785, clone 2H7, Biolegend), CD21 (BUV395, clone B-ly4, BD Biosciences), IgG (BUV737, clone G18-145, BD Biosciences), CD138 (APC, clone MI15, Biolegend), Fixable Viability Dye eFlour780 (Thermo Fisher), CD38 (PE, clone OKT10, Caprico Biotechnologies), IgA (PE-Tx red, Southern Biotech), CD19 (PE-Cy5, clone J3-119, Beckman), and CD95 (PE-Cy7, clone DX2, Biolegend). Cells were washed three times with Wash Buffer and then fixed overnight at 4° C. with the eBioscience Intracellular Fixation & Permeabilization Buffer Set (Thermo Fisher Scientific, cat #88-8824-00). After fixation, cells were centrifuged at 1,028×g for 5 minutes at 4° C. without brake and washed once with eBioscience Permeabilization Buffer and resuspended in 200 μl Wash Buffer for flow cytometry analysis using the BD Symphony platform. Data were analyzed with FlowJo version 10.

Identification and Characterization of S-Specific CD4⁺ and CD8⁺ T Cells in the Blood and Airways of Immunized Macaques

Collection procedures of blood and BAL from immunized macaques followed ACUC-approved standard operating procedures and limits. Blood was collected in EDTA tubes and PBMCs were isolated by density gradient centrifugation over ficoll as previously described (Le Nouen et al., Cell 185:4811-4825, 2022). Aliquots of PBMCs were stored in 90% FBS and 10% DMSO in liquid nitrogen. BAL were first filtered through a 100 μm filter (Corning, cat #431752) to remove gross debris from the lavages. Then, the clarified BALs were spun down for 5 minutes at 544×g at 10° C. BAL fluid was separated from the cell pellet, aliquoted, snap frozen in dry ice and stored at −80° C. for further use. BAL cells were used fresh for evaluation of the S-specific CD4⁺ and CD8⁺ T cell response in the lower airways.

T cell analysis was performed as previously described (Le Nouen et al., Cell 185:4811-4825, 2022). Briefly, PBMCs that were rested overnight or freshly collected BAL cells were plated at 2×10⁷ cells/mil in 200 μl in 96-well plates in X-VIV 15 media supplemented with 10% FBS, 1000× Brefeldin (Thermo Fisher Cat #00-4506-51) and 1000× Monensin (Thermo Fisher Cat #00-4505-51) diluted 1:1,000, CD107a and CD107b (APC, each diluted at 1:50), and stimulated with the indicated peptide pools at 1 mg/ml for 6 hours at 37° C. with 5% CO₂. Replica wells of cells that were not stimulated were used as controls. Spike peptide pools consisted of Peptivator SARS-CoV-2 Prot S1 (Miltenyi Cat #130-127-048), Peptivator SARS-CoV-2 Prot_S+ (Miltenyi Cat #130-127-312), and Peptivator SARS-CoV-2 Prot_S (Miltenyi Cat #130-127-953) covering the whole spike protein. Nucleocapsid peptide pool consisted of Peptivator SARS-CoV-2 Prot_N (Miltenyi Cat #130-126-699). After stimulation, cells were centrifuged and stained using a panel of antibodies as previously described (Le Nouen et al., Cell 185:4811-4825, 2022). The antibodies used for extracellular and intracellular staining were: CD69 (FITC, clone FN50, Biolegend), granzyme B (BV421, clone GB11, BD Biosciences), CD8a (eFluor 506, clone RPAT8, Thermo Fisher), IL-2 (BV605, MQ-17H12, Biolegend), IFNγ (BV711, clone 4S.B3, Biolegend), IL-17 (BV785, clone BL168, Biolegend), TNFα (BUV395, clone Mab11, BD Biosciences), CD4 (BUV496, clone SK3, BD Biosciences), CD95 (BUV737, clone DX2, BD Biosciences), CD3 (BUV805, clone SP34-2, BD Biosciences), CD107a (AF647, clone H4A3, Biolegend), CD107b (AF647, clone H4B4, Biolegend), viability Dye cFluor780 (Thermo Fisher), CD103 (PE, clone B-Ly7, eBioscience), CD28 (PE/Dazzle 594, clone CD28.2, Biolegend), Ki-67 (PE-Cy7, clone B56, BD Biosciences), Foxp3 (AF700, clone PCH101, Thermo Fisher). After staining, cells were washed twice with 2× permeabilization buffer (eBioscience) and resuspended in PBS supplemented with 1% FBS and 0.05% sodium azide and analyzed by flow cytometry analysis on a BD Symphony. Data were analyzed using FlowJo version 10.

### Analysis of Cytokines in the Lower Airways of Macaques

The cytokine concentration in representative BAL fluids from macaque study #2 was determined by a bead-based multiplex immunoassay as previously described (Le Nouen et al., Cell 185:4811-4825, 2022). Briefly, BAL fluids were concentrated 10-fold using Amicon concentration tubes (3 kDa cutoff, Millipore) and the concentrations of 36 cytokines from each BAL sample were evaluated in duplicate using the NHP XL Cytokine Luminex Performance Premixed Kit (Bio-Techne Corporation, Cat #FCSTM21) following the manufacturer's instructions. Data were acquired on Millipore MagPI and analyzed using the Luminex xPONENT 4.3 software. The concentration of each cytokine was expressed in pg/ml.

### Quantification of SARS-CoV-2 Genomic N and Subgenomic E by RT-qPCR

Viral RNA from nasopharyngeal swab, and bronchoalveolar lavage samples collected on day 6 post-challenge was extracted in BSL3 laboratory using the QIAamp Viral RNA Mini Kit (Qiagen) as previously described (Le Nouen et al., Cell 185:4811-4825, 2022). Homogenized lung tissue sections were mixed with Trizol LS Reagent (ThermoFisher) in a BSL3 laboratory and RNA was extracted using Phasemaker Tubes Complete System (ThermoFisher) and the PureLink RNA Mini Kit (ThermoFisher) according to the manufacturer's instructions. Purified RNA was stored at −80° C. until analysis. Then, the SARS-CoV-2 genomic N RNA and subgenomic E mRNA were quantified in triplicate using the TaqMan RNA-to-Ct 1-Step Kit (ThermoFisher) as previously described (Le Nouen et al., Cell 185:4811-4825, 2022) using previously reported TaqMan primers/probes on a QuantStudio 7 Pro real-time PCR system (ThermoFisher). Copy numbers were determined using standard curves generated using serially diluted pcDNA3.1 plasmids encoding gN or sgE sequences. The sensitivity of the qPCR assays was 10 copies, corresponding to a limit of detection of 2.8 log₁₀ copies per ml of nasopharyngeal swab, and bronchoalveolar lavage and to 3.6 log₁₀ copies per gram of lung tissue.

### Statistical Analysis

Data sets were analyzed for significance using One-way ANOVA with Tukey post-test or two-way ANOVA with Sidak's post-test on GraphPad Prism version 9.5. A log₁₀ transformation was applied to data sets when necessary to obtain comparable standard deviation among groups of values which is a necessary requirement for these tests. Data sets were only considered significantly different at P≤0.05.

### Example 3: Generation of MPV/S and MPV/S-2P and Characterization in Cell Culture

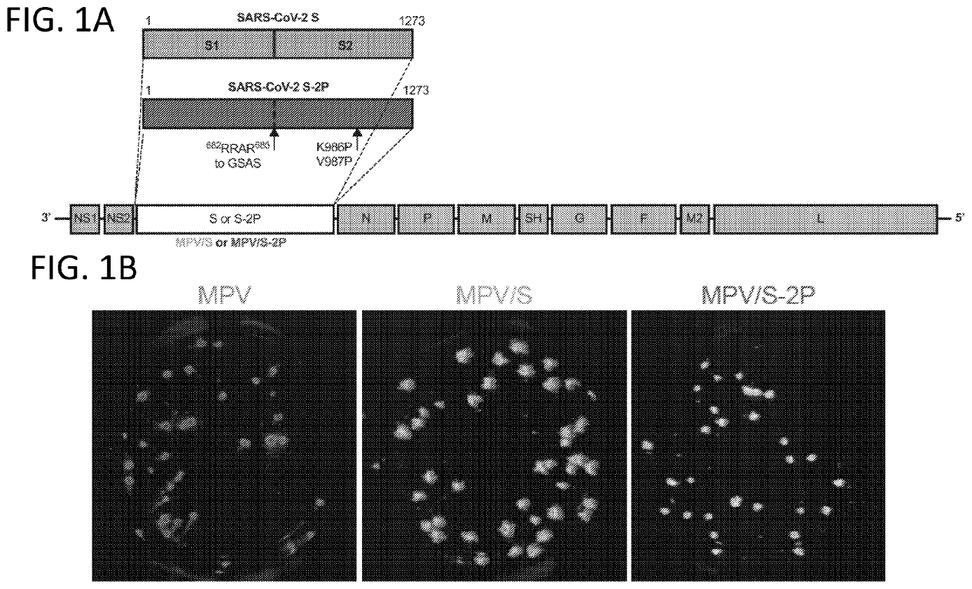

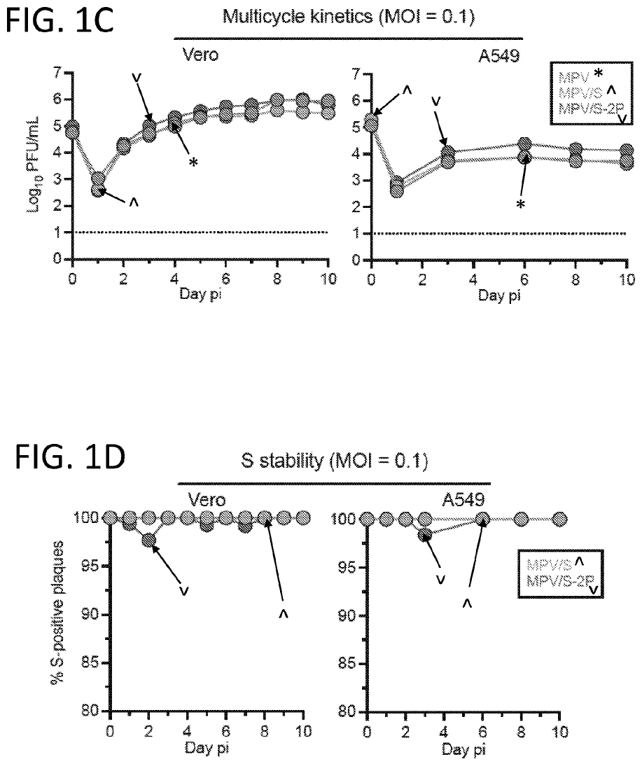

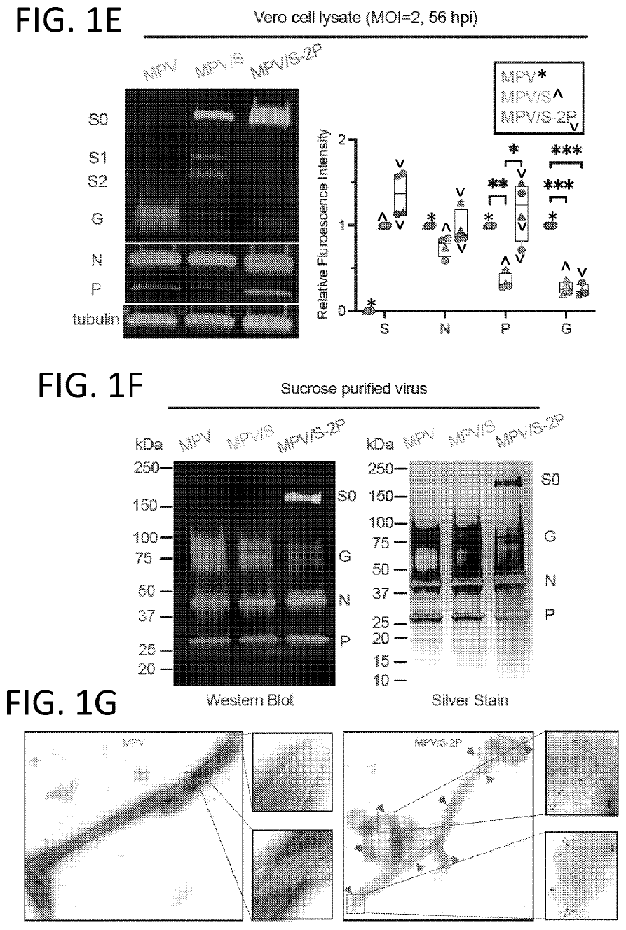

An MPV vaccine vector based on a recombinant version of MPV strain 15 was previously developed⁶. Because of concerns of possible over-attenuation due to the addition of a supernumerary gene into the MPV vector, the L ORF encoding the MPV polymerase (Brock et al., J Virol 92(17):e00723, 2018) was partially codon-pair optimized (FIG. 1A). This resulted in a modest increase in replication in mice, with a phenotype similar to recombinant wildtype MPV. To generate a version of MPV expressing the SARS-CoV-2 S protein, the ORF encoding the S protein (aa 1-1,273) of the ancestral SARS-CoV-2 S protein (GENBANK Accession No. MN908947) was codon-optimized for expression in humans. A second version of this optimized S ORF incorporated two proline substitutions ([K986P] and [V987P]) which stabilized S in the prefusion conformation, and the S1/S2 furin cleavage site was ablated (S-2P, Wrapp er al, Science 367:1260-1263, 2020), rendering the S protein non-functional for virus entry. To generate MPV/S and MPV/S-2P, the S and S-2P ORFs were placed under control of MPV transcription signals and inserted, using cDNAs of the MPV antigenome (a positive-sense copy of the genome), at the third genome position in the MPV genome, between the MPV nonstructural protein 2 (NS2) and nucleoprotein (N) genes (FIG. 1A), as described in Example 1. MPV/S and MPV/S-2P as well as the empty-vector control MPV were readily rescued by reverse genetics and amplified in Vero cells. Virus stocks were prepared and whole-genome sequencing of each stock confirmed the absence of adventitious mutations. The stability of S expression was evaluated by titration and dual-staining immunoplaque assays of each virus stock using a rabbit hyperimmune serum against MPV (Brock et al., J Virol 92(17):e00723, 2018), and a monoclonal antibody targeting the S protein (CR3022), revealing that 100% of the MPV/S and MPV/S-2P plaques expressed S (FIG. 1B).

Multicycle replication of the MPV vectors was evaluated in Vero cells, which are a suitable substrate for vaccine manufacture, and lack a functional type 1 IFN response, and A549 cells, which represent human airway epithelial cells. Following infection at an MOI of 0.1, MPV/S and MPV/S-2P replicated similarly to MPV in both cell lines (FIG. 1C), indicating that the presence of the additional gene did not affect replication. All viruses replicated to high titers in Vero cells, while replication in A549 cells was much less efficient. In both cell lines, S or S-2P expression remained stable over 10 days of replication (FIG. 1D).

To characterize viral protein expression, Vero and A549 cells were infected with MPV, MPV/S, or MPV/S-2P at an MOI of 2 (n=2 independent experiments, with duplicate wells in each experiment). Fifty-six hours after infection, cell lysates were prepared for analysis of viral protein expression by SDS-PAGE under denaturing and reducing conditions, followed by Western blotting. Quantitative imaging of infrared immunostaining showed that the S protein had accumulated to about 1.1- to 1.6-fold higher levels in MPV/S-2P-infected Vero cells than in MPV/S infected Vero cells (FIG. 1E, left and right panels). Smaller bands consistent with cleaved S1 and S2 subunits were present in MPV/S-infected cells, but not in MPV/S-2P infected cells, confirming the absence of proteolytic cleavage of S-2P. No difference in the accumulation of the MPV nucleoprotein (N) was detected between MPV, MPV/S, and MPV/S-2P infected Vero or A594 cells over time, but a 3-fold decrease in expression of the MPV phosphoprotein P in MPV/S-infected cells compared to MPV- and MPV/S-2P infected cells, respectively, was noted. Finally, a significant 4-fold lower level of expression of the MPV attachment protein G was detected in MPV/S- and MV/S-2P-infected Vero and A549 cells compared to MPV-infected cells (FIG. 1E), suggesting that S glycoprotein expression might be associated with a reduction in expression or processing of the heavily glycosylated MPV attachment protein G.

The protein content of purified virus particles was also evaluated. Aliquots from each virus stock were purified by ultracentrifugation through 30%/60% sucrose cushions, followed by SDS-PAGE and Western blotting or silver staining (FIG. 1F). No protein band consistent with S was detectable in purified MPV or MPV/S preparations, but a strong band consistent with S0 protein was identified in purified MPV/S-2P particles, suggesting that the S protein was associated with MPV/S-2P, but not with MPV/S virus particles. No apparent differences in MPV N and P content were detected between the purified virus preparations, but weaker bands of MPV G were detected in MPV/S-2P and MVP/S preparations compared to the MPV control by Western blot. Electron microscopy of purified MPV/S-2P and MPV particles showed that MPV formed long viral filaments typical of pneumoviruses. S-specific immunogold labelling was abundant on the MPV/S-2P virions, but not on MPV particles, further confirming the presence of S on MPV/S-2P particles (FIG. 1G).

### Example 4: MPV and Derivatives Replicate to Low Levels and are Safe and in Macaques; MPV/S-2P Induced a Stronger Mucosal Anti-S Antibody Response than MPV/S

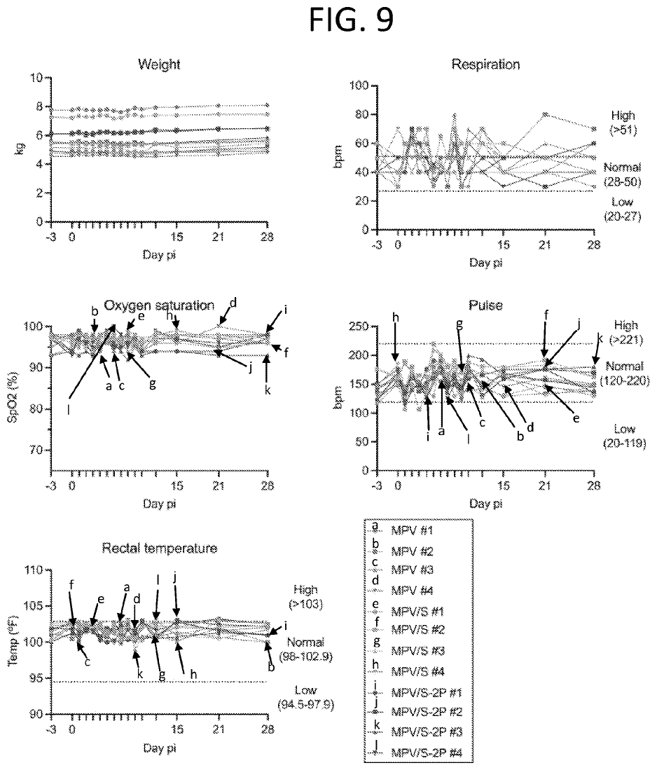

To compare the replication, safety, and immunogenicity of the MPV/S and MPV/S-2P vaccine candidates, rhesus macaques (Study 1, n=4 per group) were immunized intranasally and intratracheally (TN/IT) with 6.3 log₁₀ PFU of MPV (empty vector control), MPV/S or MPV/S-2P (FIGS. 8 and 9A). Nasopharyngeal swabs (NS) and tracheal lavages (TL) were collected over 15 days post-immunization (pi) to evaluate vaccine virus shedding in the upper airways (UA) and lower airways (UA). Shedding of MPV and derivatives were detected in the UA in all animals, but only sporadically and at low levels (≤2 log₁₀ PFU/ml, FIG. 88, left panel). In the LA, shedding of MPV and derivatives was also low and sporadic, with MPV replicating the most efficiently [geometric mean peak titers (GMT) of 2.5 log₁₀ PFU/ml on day 6 pi]; MPV/S2-P was detectable only in 3/4 animals. All macaques cleared MPV and derivatives from the UA and LA by day 8 and 12 post-immunization (pi), respectively. Thus, following IN/IT immunization, MPV replication was highly restricted in rhesus macaques, as shown previously (Brock et al., J Virol 92(17):e00723-18, 2018; Brock et al., J Virol 86:5829-5843, 2012). No changes in vital signs were observed following immunization of macaques with MPV or derivatives (FIG. 9), indicating that MPV is safe in macaques.

Nasal wash (NW) and serum were collected before immunization and on days 15, 21, and 28 after immunization to determine the mucosal and systemic antibody responses. Despite its low level of replication, MPV/S-2P induced anti-S and anti-RBD IgG and IgA responses in the UA of all four animals. GMTs in MPV/S-2P-immunized animals peaked at 21 days pi (FIGS. 8C and 8D). Compared to MPV/S-2P, mucosal antibody responses induced by MPV/S were more variable and lower (P<0.05 for anti-RBD IgG and IgA at day 21, FIGS. 8C and 8D). IN/IT immunization also induced high levels of serum anti-S and anti-RBD IgG and IgA as early as day 15 pi (FIGS. 8E and 8F). The serum anti-S and RBD IgG and IgA response induced by MPV/S-2P also was significantly stronger than that induced by MPV/S (P<0.005 to p<0.001, FIGS. 8E and 8F); serum IgG titers to S and RBD in MPV/S-2P immunized animals (GMTs of 4.8 and 5.5 log₁₀, respectively, detected on day 28 pi) were 5- and 32-fold higher, respectively, than those in plasma from 23 SARS-CoV-2 convalescent human donors (FIG. 8E). Serum antibodies from three MPV/S- and two MPV/S-2P-immunized macaques neutralized the vaccine-matched WA1/2020 strain, but the neutralizing activity was relatively low (FIG. 8G, left panel). Thus, study #1 confirmed that MPV/S-2P, expressing the prefusion-stabilized S-2P antigen, was more immunogenic than MPV/S, expressing the native version of the S protein. As expected, no anti-S mucosal or serum antibodies were induced by the empty vector control (FIGS. 8C-8F); MPV and derivatives also induced strong serum neutralizing antibodies against the MPV vector (FIG. 8G, right panel).

As a surrogate to additional BSL-3 neutralization assays, the ability of serum antibodies to inhibit binding of soluble ACE2 receptor to purified S proteins of SARS-CoV-2 variants was evaluated. Serum samples were incubated on plates coated with purified S proteins from 18 SARS-CoV-2 variants and the percentage of ACE2 binding inhibition by serum antibodies was determined (FIGS. 8H-8I). The strongest ACE2 binding inhibition by serum antibodies was detected for the vaccine-matched S protein derived from strain WA1/2020, with peak median inhibition rates of 94% and 97% for sera from MPV/S and MPV/S-2P immunized animals, respectively, on day 21 post-immunization (FIG. 8H). Serum ACE2 binding inhibition was also detected against S proteins of the alpha, beta, delta, and French variants with median inhibition rates on day 21 ranging from 43-83% for MPV/S and 62-92% for MPV/S-2P (FIGS. 8H-8I). ACE2 binding inhibition for S proteins from 13 different Omicron variants was also evaluated. However, the inhibition rates were low, with medians ranging from 0-23% for sera collected on day 21 following immunization with MPV/S or MPV/S-2P, only slightly higher than the background in empty-vector control immunized animals (median 0-16%) (FIGS. 8H-8I). Overall, these results confirmed that MPV/S-2P, expressing the prefusion-stabilized S-2P antigen, was more immunogenic than MPV/S, expressing the native version of the S protein.

### Example 5: A Prime/Boost Regimen of MPV/S-2P Increased the Magnitude and Breadth of the Mucosal Anti-S Antibody Response in the Airways, and Induced High Levels of Dimeric Anti-S IgA

Since MPV/S-2P was more immunogenic than MPV/S, it was selected for further evaluation. To evaluate if the mucosal and serum immune response, including the serum neutralizing antibody response to SARS-CoV-2, and the protective efficacy against SARS-CoV-2 challenge could be improved by a second dose of vaccine, a prime-boost study (study #2) was designed which included three groups of macaques (n=4 per group, see FIG. 10 for study design). Group 1 received a single IN/IT dose of MPV (empty-vector control), and groups 2 and 3 received MPV/S-2P (6.3 log₁₀ PFU per animal). Results from MPV/S2-P primed animals from groups 2 and 3 were combined for analysis (n=8). Twenty-eight days after the first dose, the four animals of group 3 received a second IN/IT dose of MPV/S-2P (boost, 6.3 log₁₀ PFU).

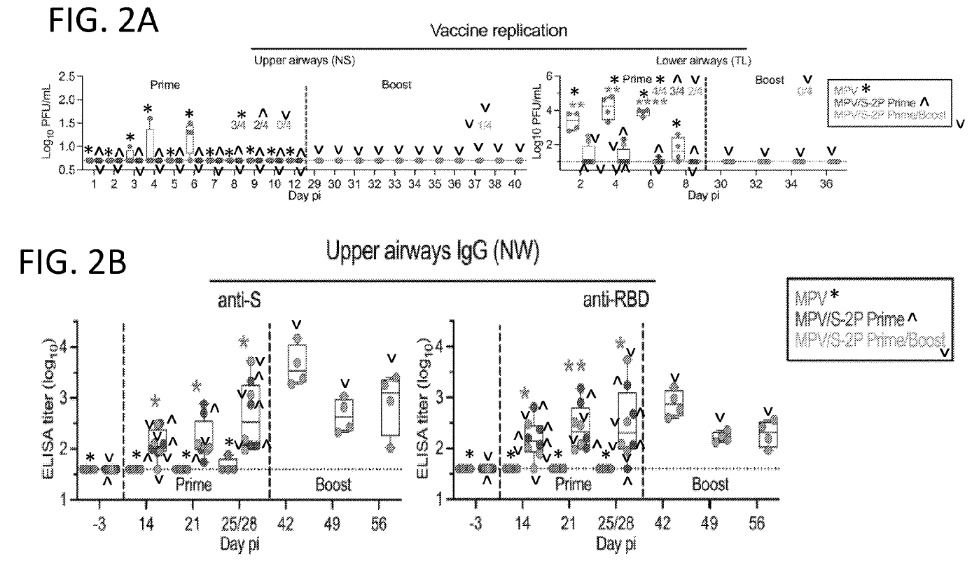

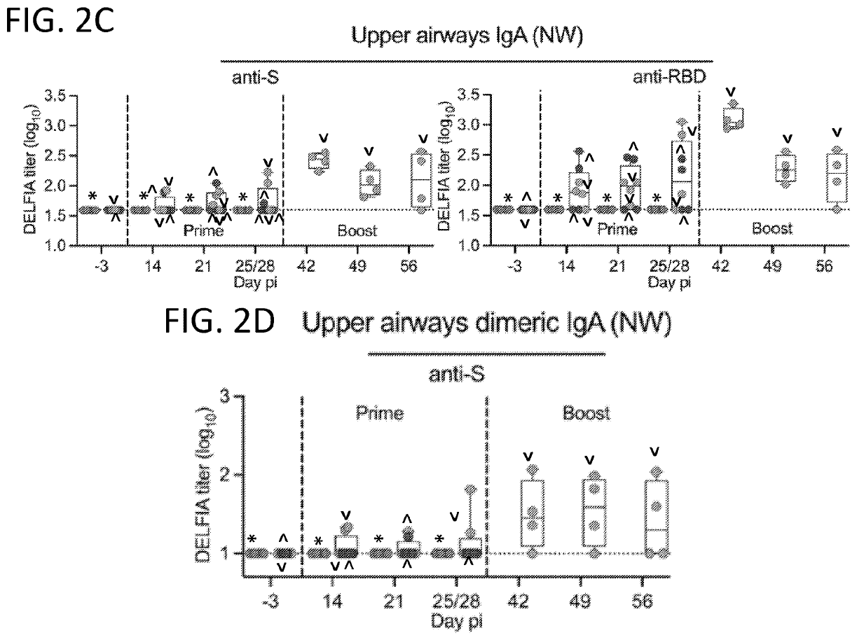

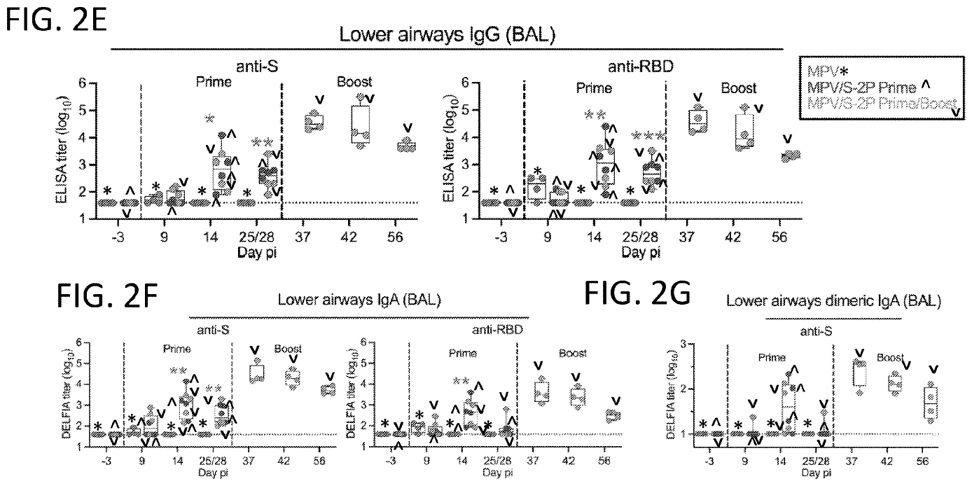

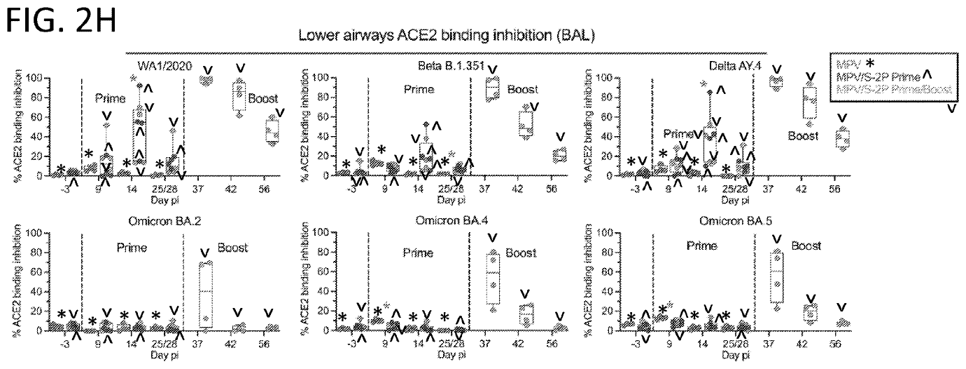

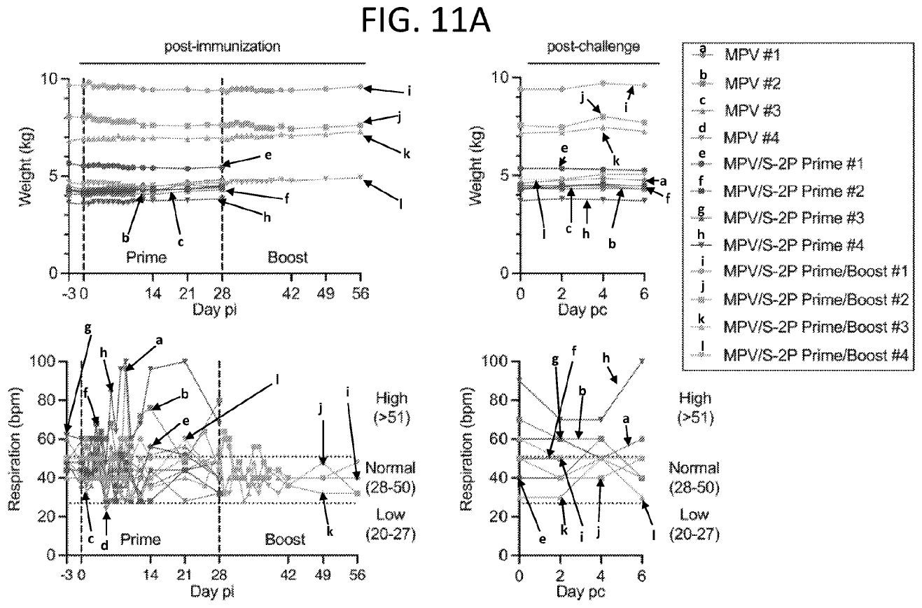

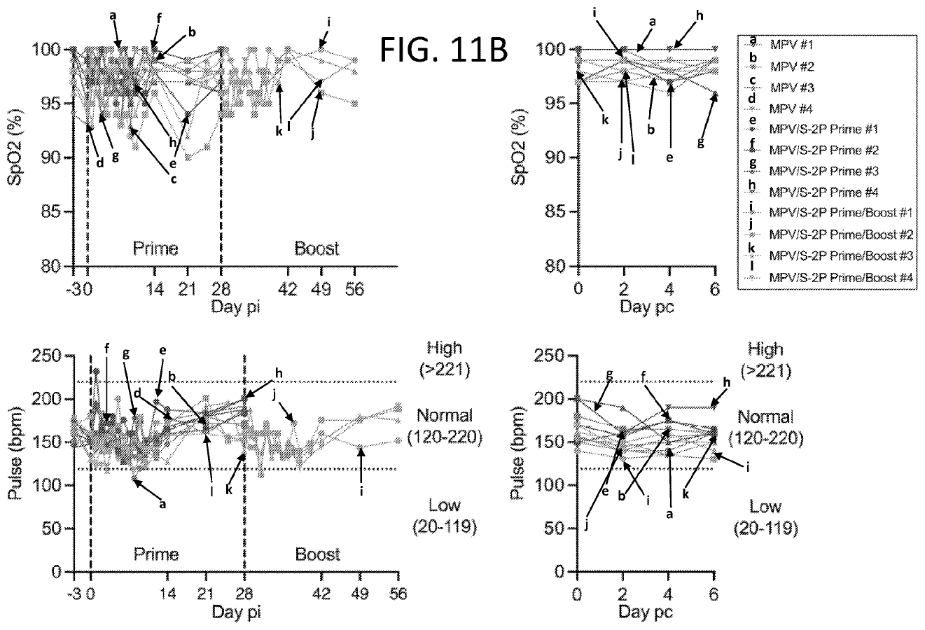

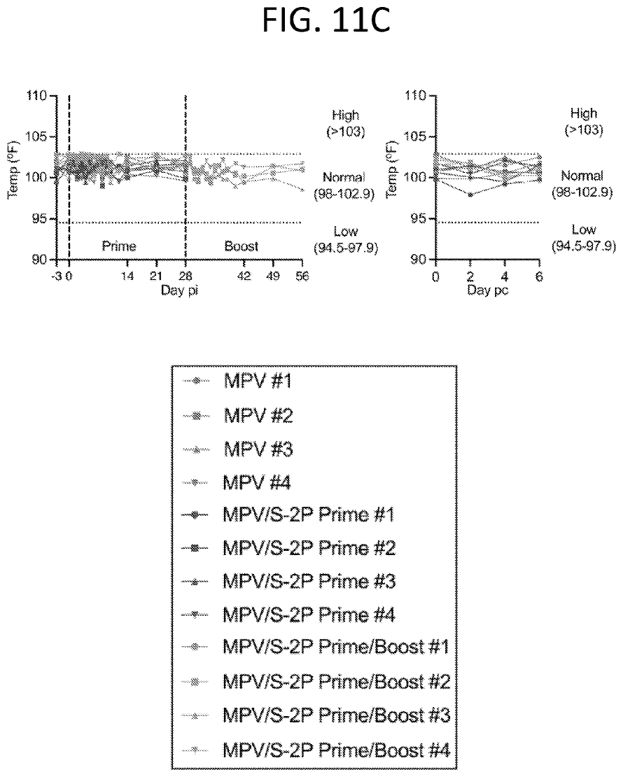

Similar to the first study, NS and TL were collected on indicated days to characterize vaccine virus shedding and replication. Analysis of NS specimens obtained after the first dose showed that shedding of the MPV empty-vector control from the UA was low (<2 log₁₀ PFU/ml, detectable in 3/4 animals) and sporadic; shedding of MPV/S-2P was detected in the UA of 2/8 animals only, with titers at the limit of detection (FIG. 2A, left panel). In the LA, replication of MPV empty-vector control was more robust than in the UA, with virus detectable in all four animals of group 1 over 8 days, and peak titers on day 4 pi (median 4.3 log₁₀ PFU/ml; n=4 animals); MPV/S-2P vaccine virus was detected in the LA of 3/4 and 2/4 macaques in group 2 and 3, respectively, albeit only through day 6, and titers were significantly lower compared to those of the MPV empty-vector control (P<0.01 and P<0.0001, FIG. 2A, right panel). In group 3 that received a second dose of MPV/S-2P vaccine (boost; n=4), shedding of MPV/S-2P was detected only in a single animal in the UA on day 3 at the level of detection of the assay (FIG. 2A). As in the first study, no changes of the macaques' vital signs were observed following IN/IT immunization with MPV or MPV/S-2P (FIG. 11).

As observed in the first study, a single TN/IT dose of MPV/S-2P efficiently induced mucosal anti-S and anti-RBD IgG and IgA in the UA, detectable in NW by ELISA (FIGS. 2B and 2C, n=8 animals). Despite vaccine replication being undetectable in the UA, the second dose of MPV/S-2P further boosted the mucosal antibody response, with a 2.5- to 9.4-fold increase of the anti-S and RBD mucosal IgG and IgA GMTs in the UA from day 25/28 to day 42 (two weeks post-boost) in the four animals from group 3. While the first dose induced dimeric anti-S IgA in two of 8 MPV/S-2P immunized animals, the second dose induced dimeric IgA in 3 of 4 animals two weeks post-boost (FIG. 2D). In the LA, strong anti-S and anti-RBD IgG and IgA responses were detected after the first dose of MPV/S-2P (n=8), and rapid and impressive 79- to 97-fold increases in GMTs as early as day 37, which was 9 days after the second dose (n=4) (FIGS. 2E and 2F). Even though titers gradually decreased, by four weeks after the boost, anti-S IgA and IgG GMTs in the UA and the LA were still above the peak GMTs after the first dose. In the LA, MPV/S-2P also induced dimeric anti-S IgA, detectable in 6 of 8 animals on day 14 after priming, and in 4 of 4 animals on day 9 post-boost (FIG. 2G). This is notable because dimeric IgA is highly functional for virus neutralization in the respiratory mucosal environment.

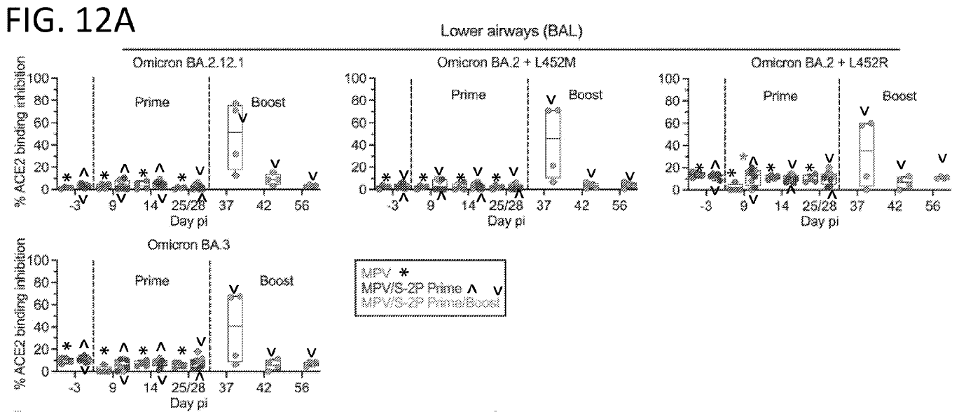

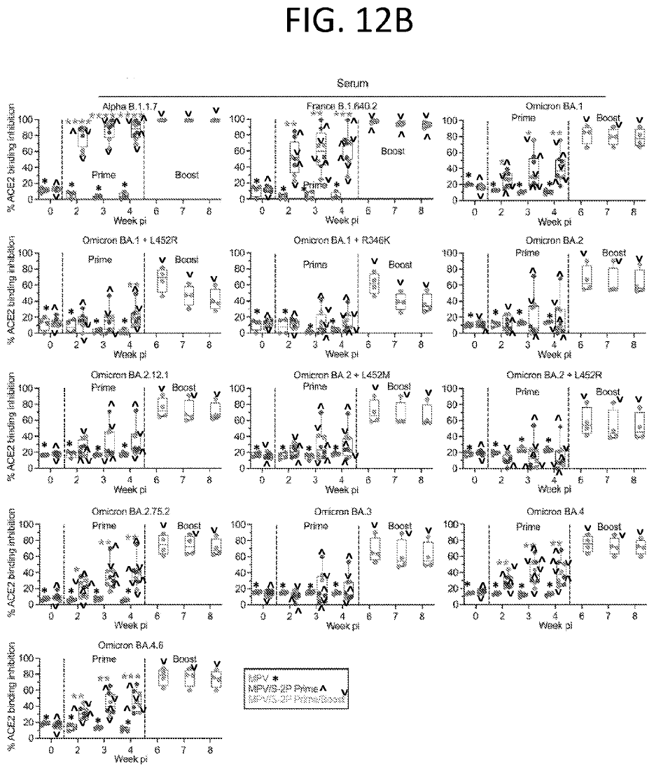

To evaluate the ability of mucosal antibodies induced by MPV/S-2P immunization to neutralize the vaccine-matched SARS-CoV-2 strain as well as variants of concerns (VoCs), an angiotensin-converting enzyme 2 receptor (ACE2) binding inhibition assay, a surrogate for a BSL3 neutralization assay, was used. This assay evaluated the ability of mucosal antibodies from NW and bronchoalveolar lavage (BAL) to inhibit binding of soluble, tagged ACE2 to purified S proteins from the vaccine-matched WA1/2020 isolate and 9 different SARS-CoV-2 variants (FIGS. 2H and 12A). NW samples displayed a high background in this assay, and thus were not further investigated. Mucosal antibodies from BAL collected on day 14 post-priming (n=8) moderately inhibited ACE2 binding to S derived from WA1/2020, Beta (B.1.351) and Delta (B.1.617.2) (median % ACE2 binding inhibition ranging from 16.6-54.8%) but did not inhibit ACE2 binding to S derived from Omicron variants (FIGS. 2H and 12A). However, BAL samples collected as early as 9 days after the MPV/S-2P boost (n=4) displayed an increased ability to inhibit the ACE2 binding to S from WA1/2020, Beta, and Delta (median % ACE2 binding inhibition: 90.2-99.2%), as well as S from Omicron sub-lineages (median % ACE2 binding inhibition: 35.2-60.8%) (FIGS. 2H and 12A). Thus, the second dose of MPV/S-2P induced a remarkable increase in the magnitude and breadth of the mucosal anti-S antibody response.

### Example 6: a Prime/Boost Regimen of MPV/S-2P Also Increased the Magnitude, Avidity, and Breadth of the Anti-S Serum Antibody Response

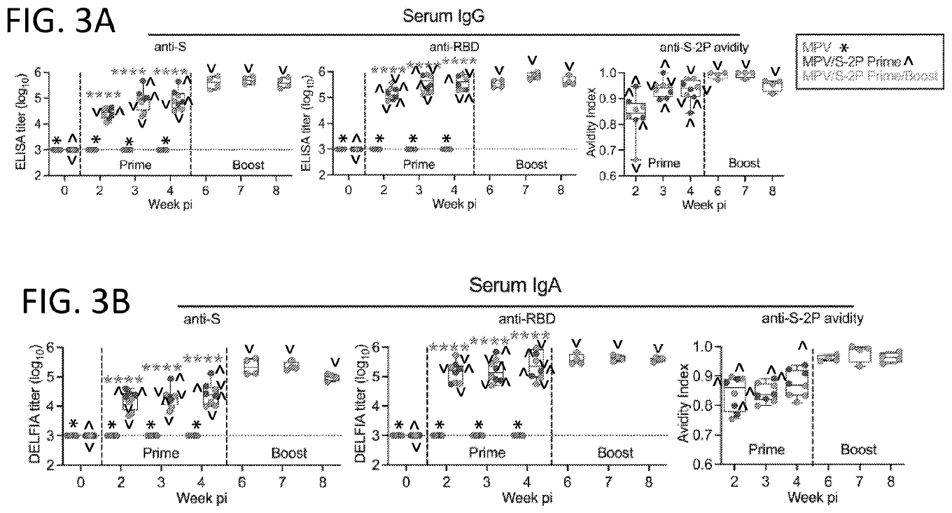

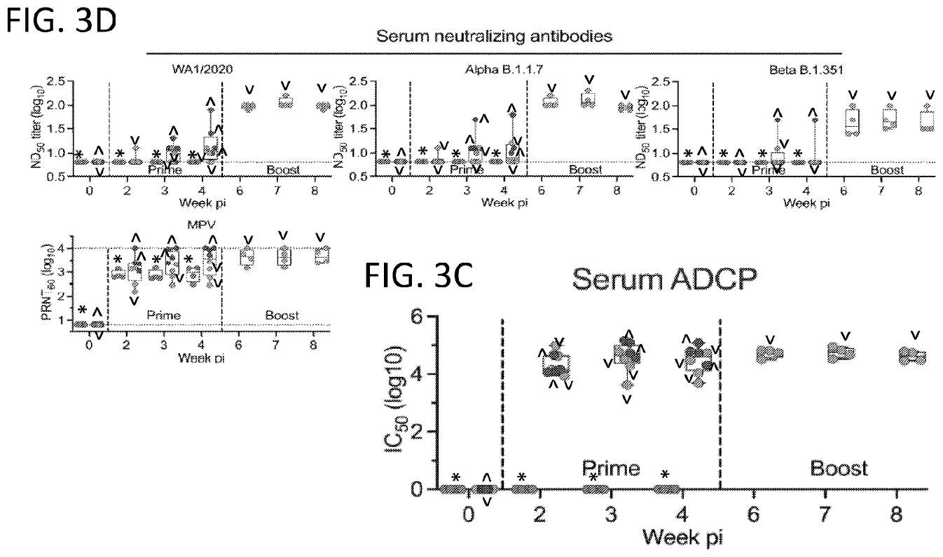

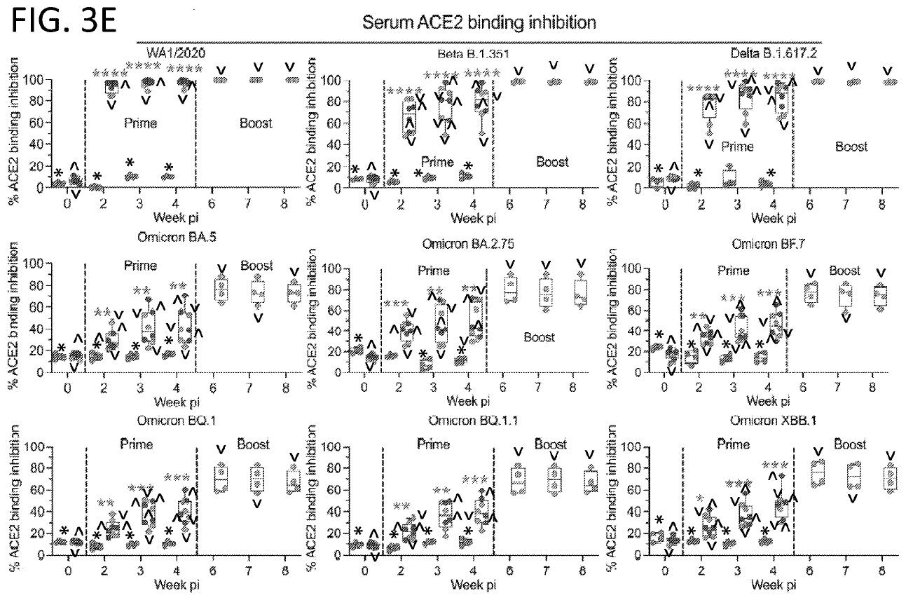

Similar to the first experiment, MPV/S2-P immunization also induced a strong systemic antibody response to the S protein. Specifically, after a single dose of MPV/S-2P, high titers of serum anti-S and anti-RBD IgA and IgG were detected and remained high through week four after priming (FIGS. 3A and 3B, n=8). The MPV/S-2P boost further increased the serum anti-S IgG and IgA titers (5.6- and 8.6-fold increase, respectively; n=4), with no significant decrease through week 4 post-boost. The strength of antibody binding was measured in an avidity assay using a further-stabilized version S-6P of the vaccine-matched S protein, and the results showed that the geometric mean of the serum anti-S avidity index increased from 0.87 and 0.94 at 4 weeks after prime to 0.98 and 0.99 at two weeks after the boost for IgA and IgG, respectively (FIGS. 3A and 3B), suggesting that further antibody maturation occurred after the boost. Furthermore, the serum anti-S antibodies also conferred antibody-dependent cellular phagocytosis (ADCP) activity after priming which became more homogenous among macaques after the boost (FIG. 3C).

To evaluate the breadth of the serum neutralizing antibody response, BSL3 neutralization assays against the vaccine-matched strain WA1/2020 or representatives of Alpha or Beta lineages of SARS-CoV-2 were also performed. As observed in the first study, neutralizing antibody titers to the vaccine-matched strain (WA1/2020) were variable and relatively low among macaques after the prime [detectable in 6/8 macaques, median 50% SARS-CoV-2 neutralizing antibody titers (ND₅₀) of 1.1 log₁₀], but a strong increase (about 9-fold) was detected in 4/4 macaques after the boost (median ND₅₀ of 2.0 log₁₀) (FIG. 3D). This suggests that despite the low or undetectable neutralizing activity after the first dose, animals were effectively primed for an anamnestic B cell response. A 13- and 7-fold increase of serum neutralizing titers against the Alpha and Beta isolates, respectively (FIG. 3D) was also detected after MPV/S-2P boost. As expected, no serum anti-S or anti-RBD IgA or IgG or S-specific ADCP activity were induced by the empty vector control (FIGS. 3A-3C), but all macaques developed high serum neutralizing antibody titers against the MPV vector that remained high in the MPV/S-2P-boosted macaques (FIG. 3D).

To determine whether the mucosal boost increased the breadth of S-specific serum antibodies, the ability of macaque sera to inhibit the binding of soluble, tagged ACE2 to purified an ACE2 binding assay was evaluated using purified S proteins of 22 different SARS-CoV-2 variants (FIGS. 3E and 12B). Sera from MPV/S-2P-primed macaques (n=8) effectively inhibited ACE2 binding to the S protein of the vaccine match, WA1/2020 (median inhibition of 98.2%), and binding inhibition further increased after boost (median inhibition of 99.8%). Inhibition of binding to S proteins derived from Alpha, Beta, Delta and B.1.640.2 (France) lineages by sera from MPV/S-2P primed animals was similarly high (median ACE2 binding inhibition of 69.5-88.4%) and further increased post-boost (median inhibition of 97.4-99.4%). Inhibition of ACE2 binding to S proteins from Omicron BA.1, BA.2, BA.3, BA.4 and derivatives by sera from MPV/S-2P primed animals was variable, ranging from 8.6-43.2%. However, sera from MPV/S-2P boosted macaques (n=4) exhibited a marked increase in inhibition of ACE2 binding (median inhibition ranging from 53.8-85.3%). Inhibition of ACE2 binding to S proteins from Omicron BA.5 and derivatives (BF.7, BQ.1, BQ.1.1, and XBB.1) after prime was modest (n=8, median inhibition of 38.2-47.2%), but was highly effective after boost (n=4, median inhibition of 66.5-77.8%). Thus, the rate of inhibition of ACE2 binding to S proteins mediated by serum antibodies increased over weeks 2-4 post-prime, and further increased post-boost, peaking, for all 4 animals, at two weeks post-boost. As expected, no inhibition of ACE2 binding was detected with sera from the four MPV-immunized macaques. Thus, a prime/boost regimen of MPV/S-2P induced high levels of serum anti-S and anti-RBD antibodies with high avidity, breadth and ADCP activity.

### Example 7: MPV/S-2P Induced S-Specific Peripheral Blood B Cells that Bind the RHD as Well as Other Regions of the Prefusion Version of the SARS-CoV-2 S Protein

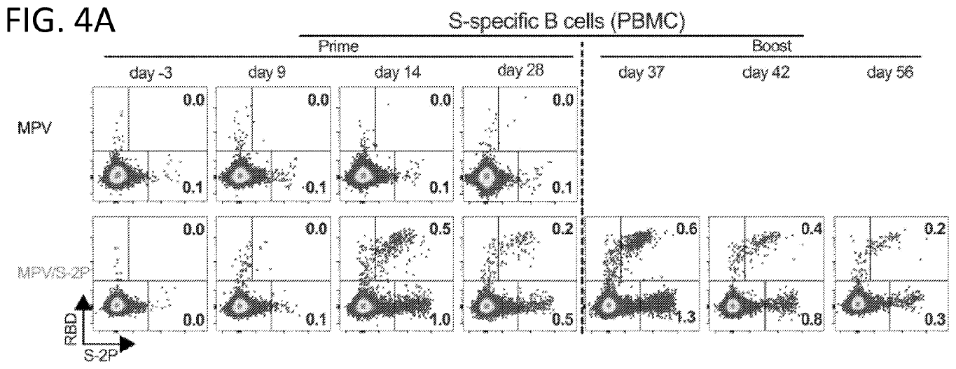

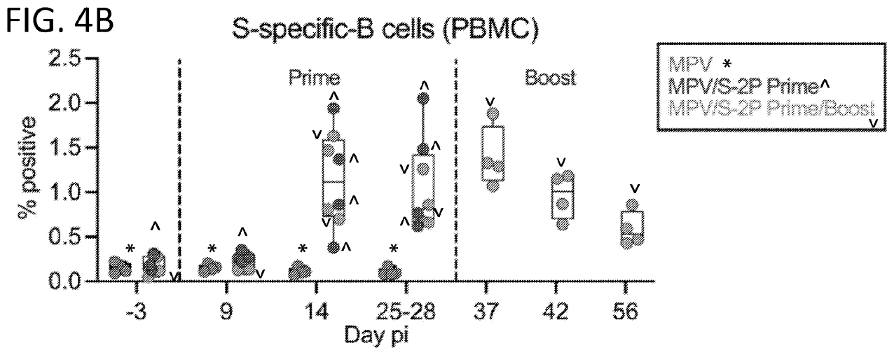

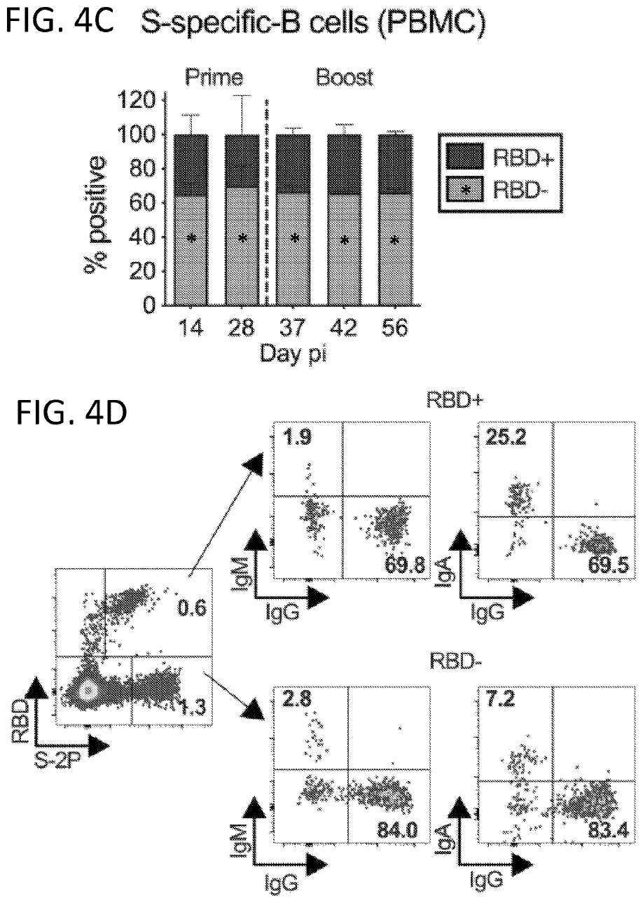

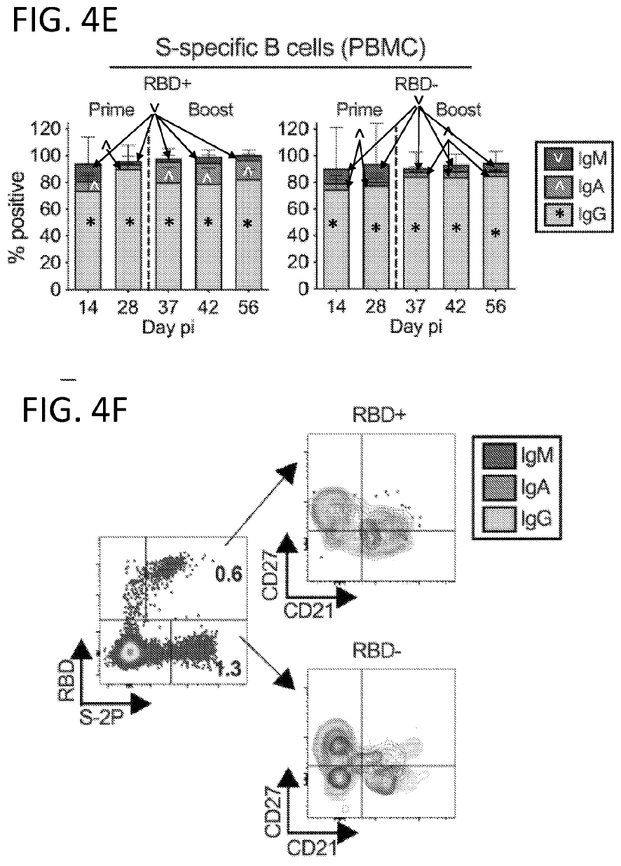

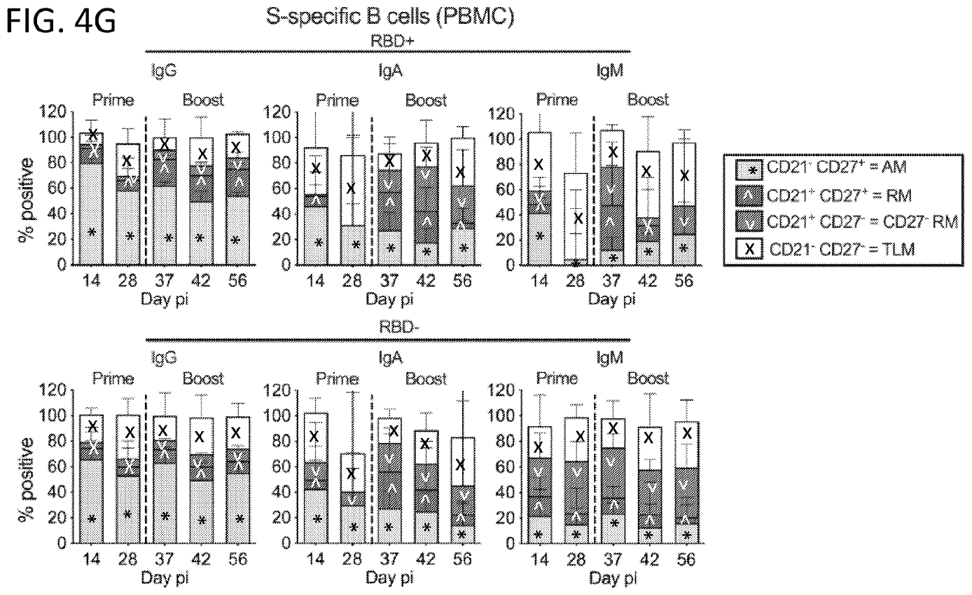

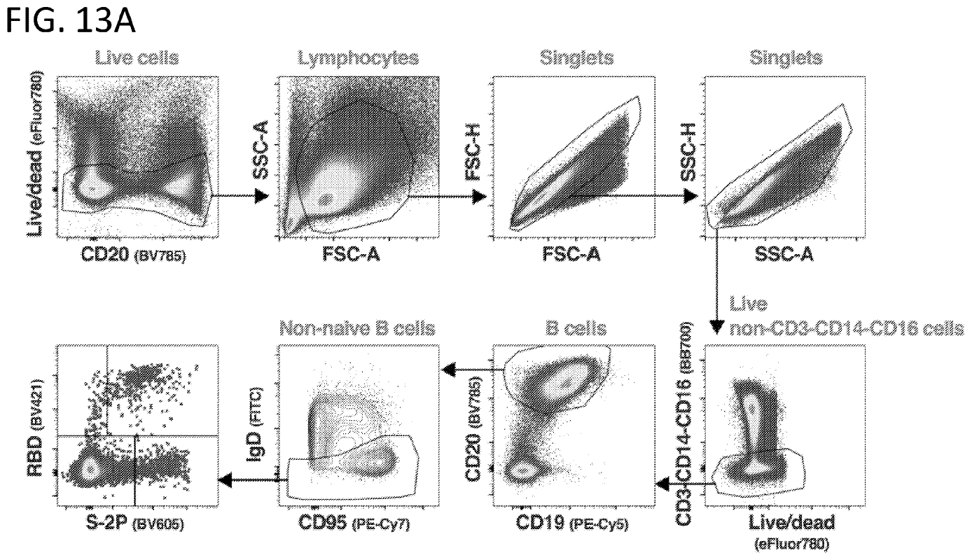

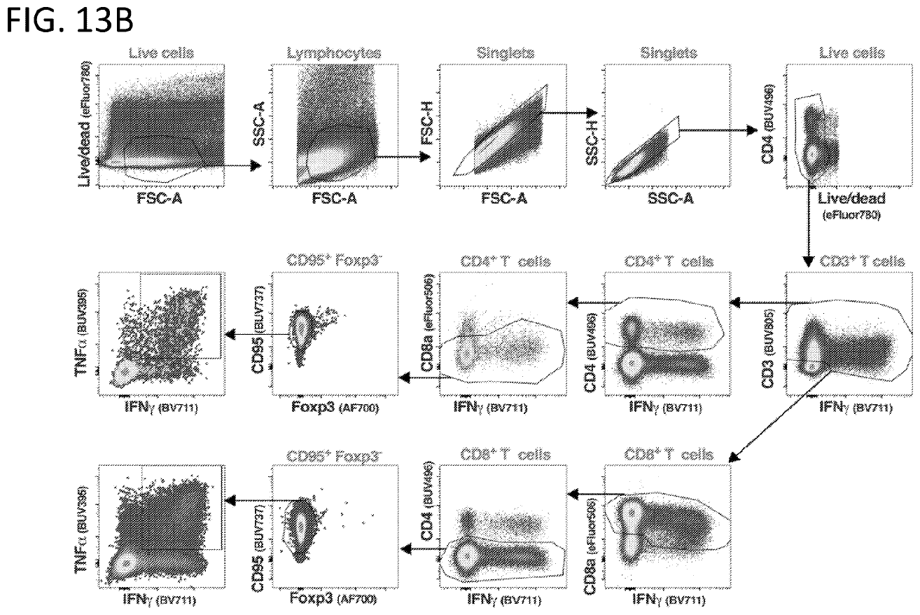

To extend the analysis of the systemic S-specific immune response following IN/IT immunization of macaques with MPV/S-2P, the kinetics and phenotype of the S-specific peripheral blood B cells post prime and boost were characterized (FIG. 4). Peripheral blood mononuclear cells (PBMCs) were stained with fluorochrome-labelled RBD and S-2P protein probes to identify S-specific B cells that recognize epitopes of the RBD (RBD⁺/S-2P⁺), or outside the RBD (RBD⁻/S-2P⁺). The cells were further stained using a cocktail of fluorochrome-labeled monoclonal antibodies and analyzed by flow cytometry (FIG. 4A). Analysis was done on live, single, non-naïve (IgD−) CD95^{+/-} B cells from PBMC collected before immunization and on six time points after prime (groups 2 and 3 combined; n=8) or boost (group 3, n=4; FIG. 10, see FIG. 13A for the gating strategy).

After MPV/S-2P prime, the peak frequency of S-specific (RBD⁺ and RBD⁻ combined) non-naïve B cells in the blood was reached by day 14 post-prime (median 1.1%). The frequency slightly declined by day 28 post-prime (median 0.8%). By day 9 after MPV/S-2P boost, a rapid recall response was detected, with median frequencies of S-specific B cells of 1.3%, followed by a steady decrease through day 28 post-boost (n=4, median 0.5% on day 28 post-boost, FIG. 4B). At each time point, RBD⁺-B cells represented approximately one third of S-specific B cells, with the remaining two thirds (RBD⁻/S-2P⁺) exhibiting specificity to undetermined epitopes of S-2P (FIG. 4C).

The majority of S-specific RBD⁺ and RBD⁻ specific B cells had an IgG isotype (80% median frequency) regardless of the time post-immunization (FIGS. 4D and 4E). However, populations of S-specific IgM and IgA B cells were detected in the blood as well. The frequency of S-specific IgM B-cells was highest at day 14 post-prime (median 12.5% of all S-specific B cells; n=8 macaques) and decreased thereafter, suggestive of isotype switching over time. S-specific IgA B cells also were detected at each time point. After MPV/S2-P boost, S-specific RBD⁺ IgA B cells (FIG. 4E) were about 4-fold more abundant than RBD⁻ IgA B cells, representing about 16% and 4% of the S-specific B cells, respectively (n=4 animals). About 60% of the S-specific IgG B cells exhibited an activated memory (AM) phenotype (CD21⁻/CD27⁺), with similar proportions for RBD⁺ and RBD⁻ cells (FIGS. 4F and 4G, left). The phenotypes of the S-specific IgA and IgM B cells were more variable, with a lower proportion of activated memory B cells, an increased proportion of cells with a tissue-resident like memory phenotype (CD21⁻/CD27⁻), and, especially after the boost, resting memory phenotype (CD21⁺/CD27⁺ or CD21⁺/CD27⁻) (FIG. 4G, center and right). Thus, MPV/S-2P induced S-specific B cells in the blood that target both the RBD as well as epitopes on other regions of S, and a recall response of mostly activated memory IgG B cells occurred rapidly after boost.

### Example 8: Priming with MPV/S2-P by the IN/IT Route Induces S-Specific CD4⁺ and CD8⁺ T Cells in the Blood and Airways that were Reactivated after Boost

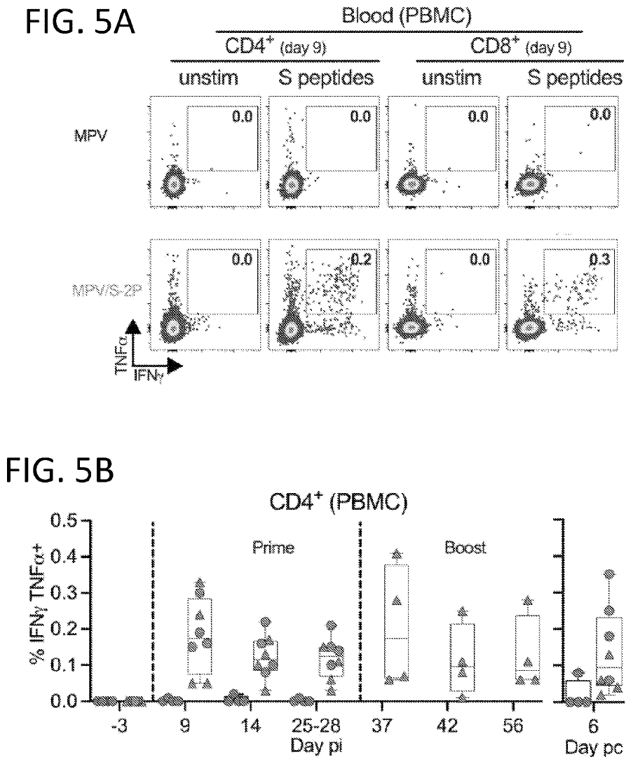

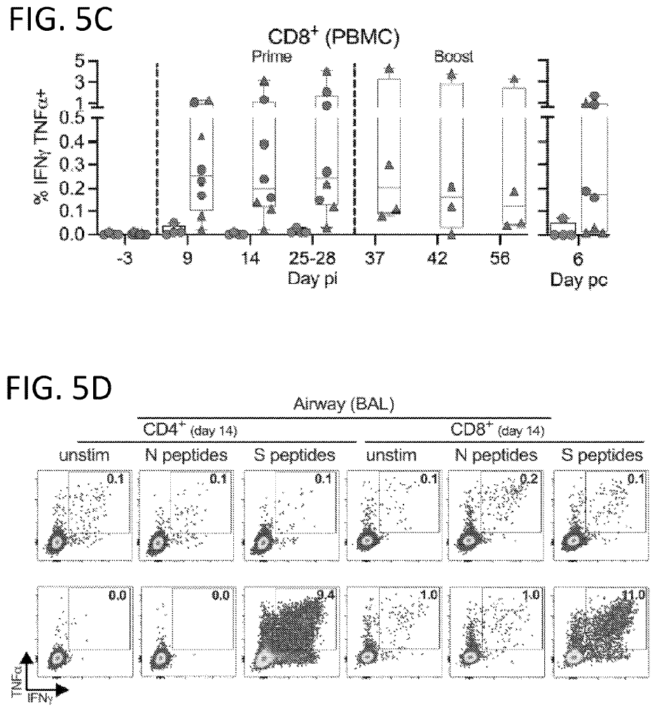

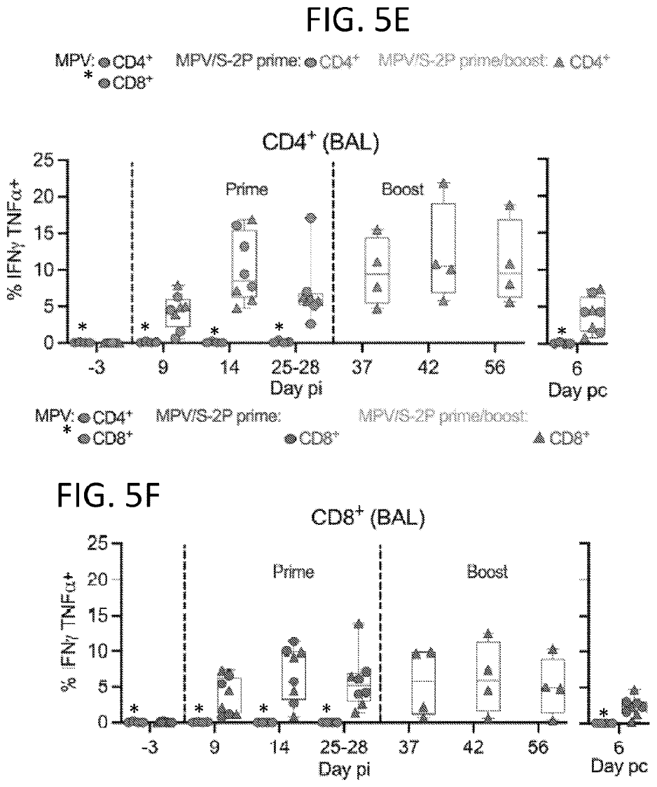

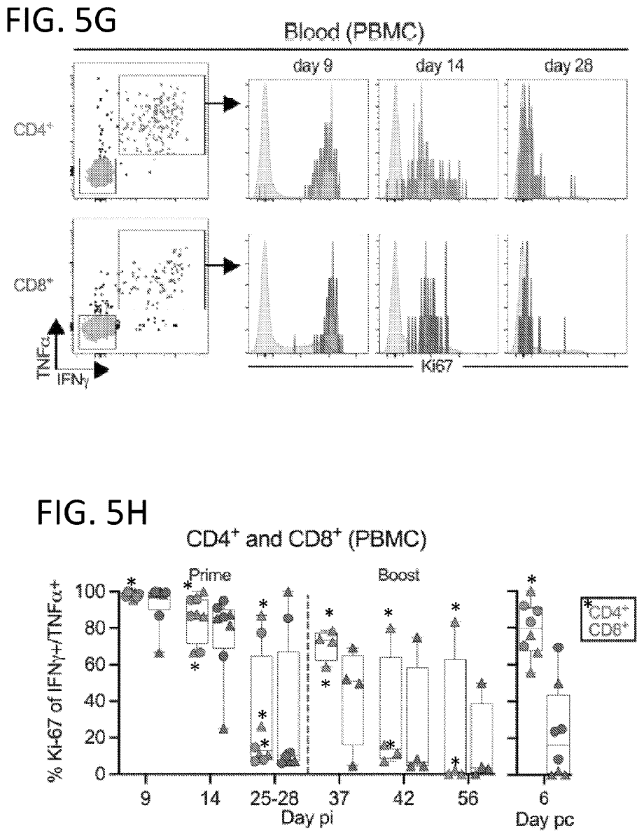

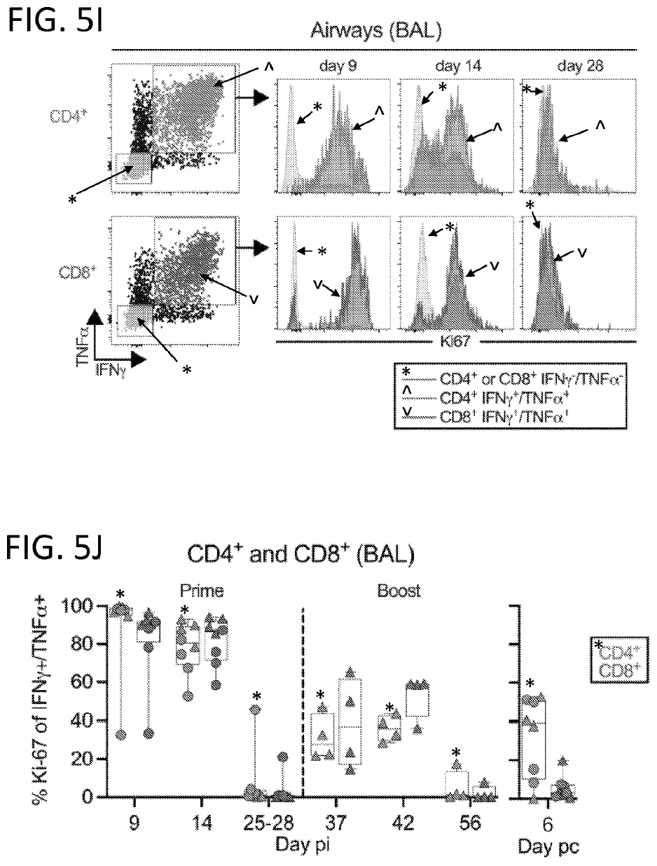

It was investigated whether IN/IT immunization with MPV/S-2P induced S-specific CD4⁺ and CD8⁺ T cells in the blood and airways following TN/IT immunization with MPV/S-2P (FIG. 5). To do so, aliquots of PBMCs and mononuclear cells collected from BAL at the indicated times before immunization or post-prime or post-boost were left unstimulated or were stimulated with pools of overlapping peptides covering the entire S (PBMC and BAL) or N (BAL only) derived from the vaccine-matched Wuhan-1 strain. Unstimulated and stimulated cells were stained using a cocktail of fluorochrome-labeled monoclonal antibodies and analyzed by flow cytometry to characterize the phenotype of the S-specific T cells. Gating was done on live, single, non-naïve (CD95+), non-regulatory (FoxP3⁻), CD4⁺ or CD8⁺ T cells (see FIG. 13B for the gating strategy). Data from representative macaques are shown in FIGS. 5A, 5D, 5G and 5I, while data from all macaques are shown in FIGS. 5B, 5C, 5E, 5F, 5H and 5J (post MPV-prime: n=4, post-MPV/S-2P prime: n=8; post-MPV/S-2P boost: n=4).

The induction of S-specific IFNγ⁺/TNFα⁺ CD4⁺ T cells in the blood that peaked on day 9 after prime (median 0.2%) and declined by day 25/28 (median 0.1%) but CD4⁺ T-cells were restimulated 9 days after boost (median 0.2%, FIGS. 5A-5B). The frequency of S-specific IFNγ⁺/TNFα⁺ CD8⁺ T cell in the blood was variable over the course of the experiment, but all macaques had measurable S-specific IFNγ⁻/TNFα⁺ CD8⁺ T cells at each day pi (median 0.12-0.21%, FIGS. 5A and 5C).

In the airways, MPV/S-2P induced a remarkable response of S-specific IFNγ⁺/TNFα⁺ CD4⁺ and CD8⁺ T cells (FIGS. 5D to 5F) that peaked on day 14 post-prime (medians 8.6% and 7.5% for CD4⁺ and CD8⁺ T cells, respectively) and then decreased by day 25/28 post-prime (medians 6.0% and 5.2% for CD4⁺ and CD8⁺ T cells, respectively). However, the MPV/S-2P boost restimulated the IFNγ⁺/TNFα⁺ S-specific T cells to high levels that remained relatively steady between days 9-28 post-boost (medians of 10.5% and 6.0% for CD4⁺ and CD8⁺ T cells on day 14 post-boost, respectively). As expected, CD4⁺ and CD8⁺ T cells from BAL were not stimulated by the pool of N peptides (FIG. 5D), and the empty MPV vector did not induce any S-specific T cells in the blood and airways (FIGS. 5A to 5F).

S-specific IFNγ⁺/TNFα⁺ CD4⁺ and CD8⁺ T cells in both the blood and BAL of MPV/S-2P-immunized macaques expressed high levels of the Ki-67 proliferation marker on day 9 post-prime that then steadily decreased until day 28 post prime (FIGS. 5G to 5J). However, the MPV/S-2P boost substantially increased Ki-67 expression, indicating restored proliferation of S-specific T cells in both blood and airways as early as day 37, which is day 9 post boost (FIGS. 5H and 5J).

### Example 9: MPV/S-2P Immunization Induced S-Specific T Cells that Transitioned to a Tissue-Resident Memory Phenotype in the Airways

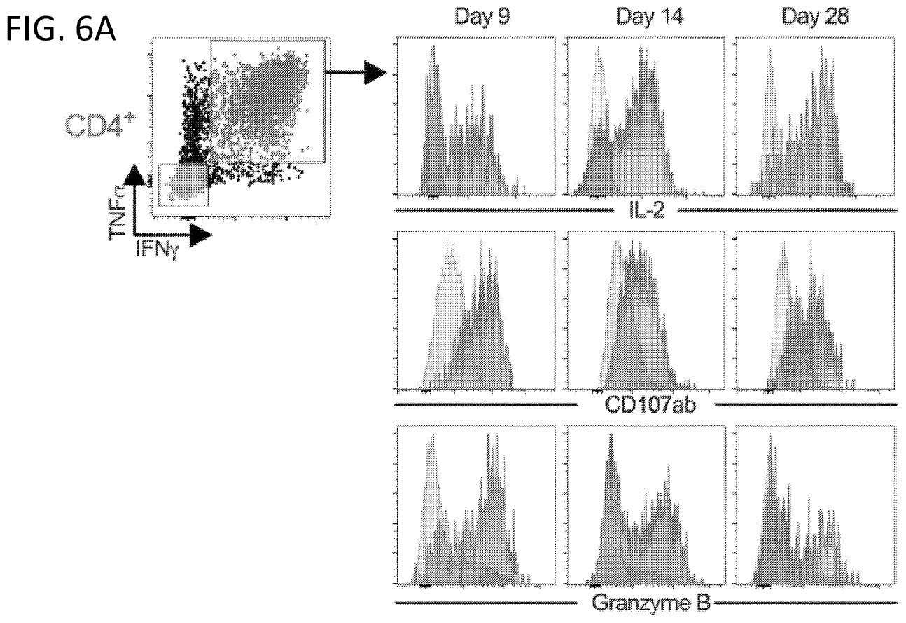

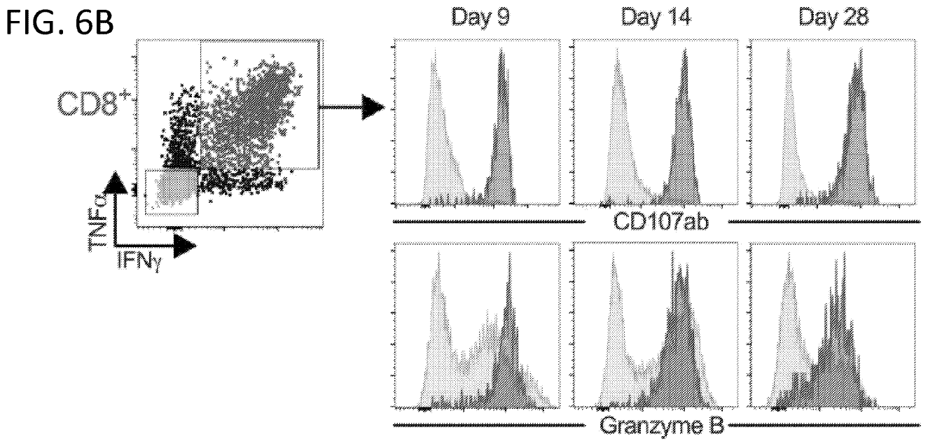

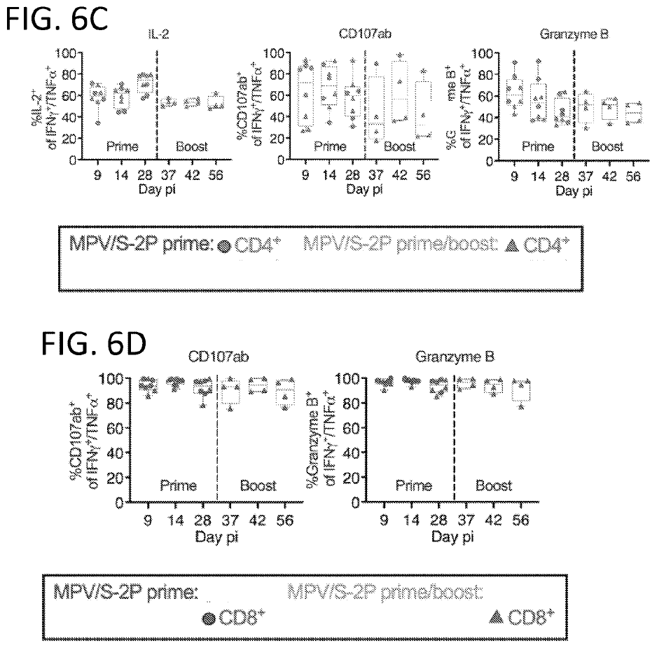

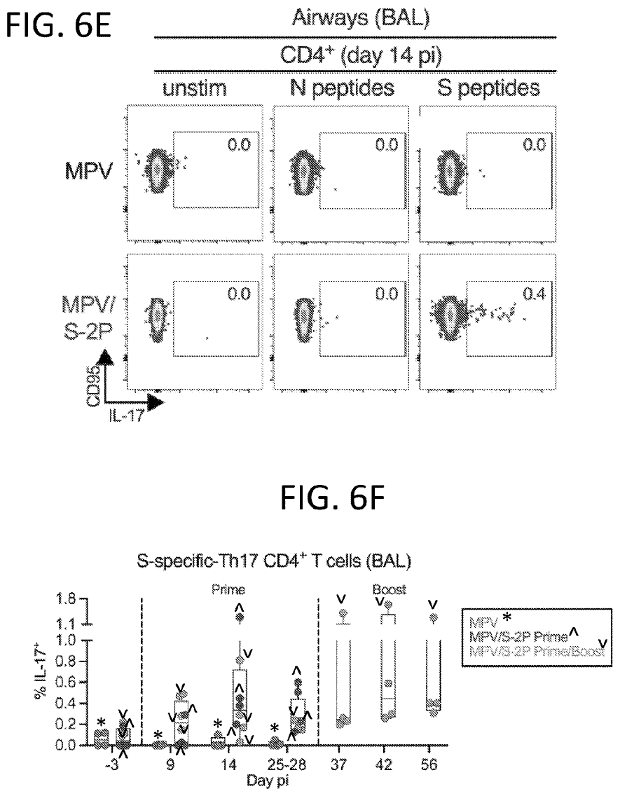

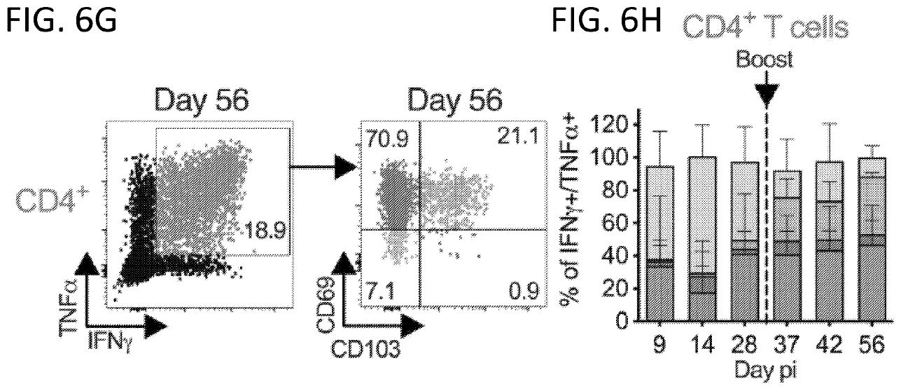

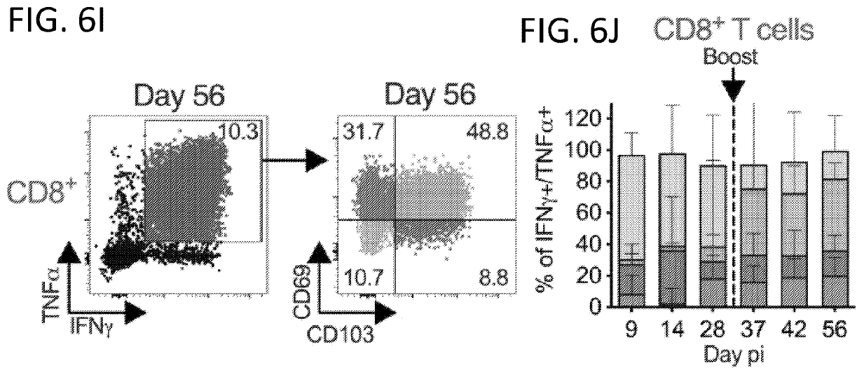

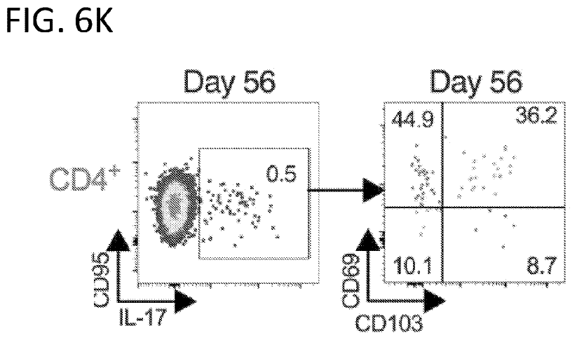

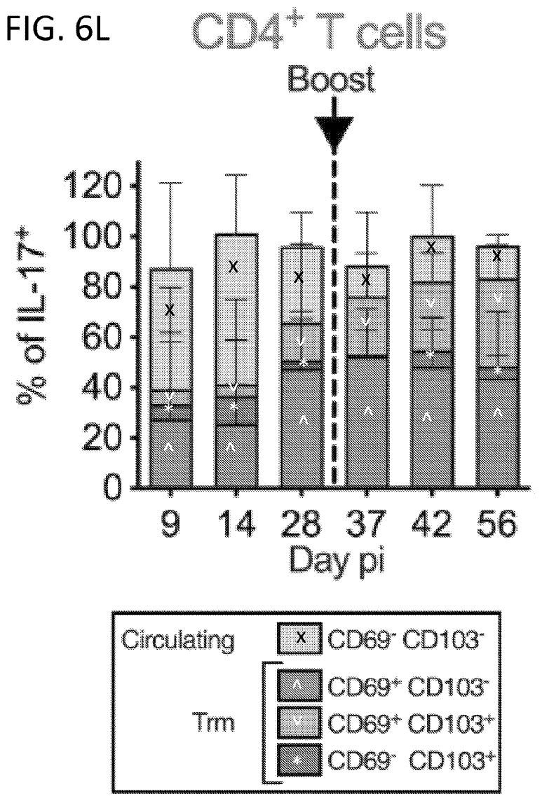

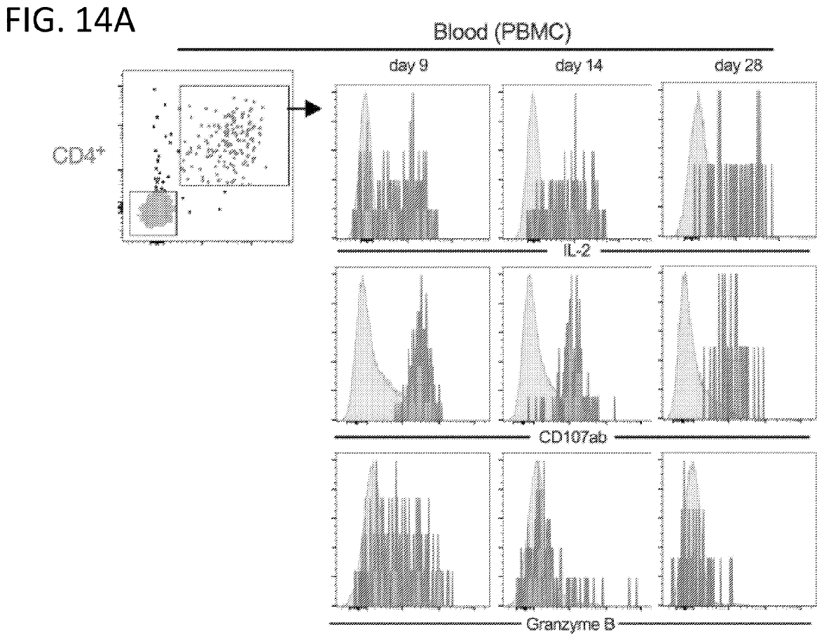

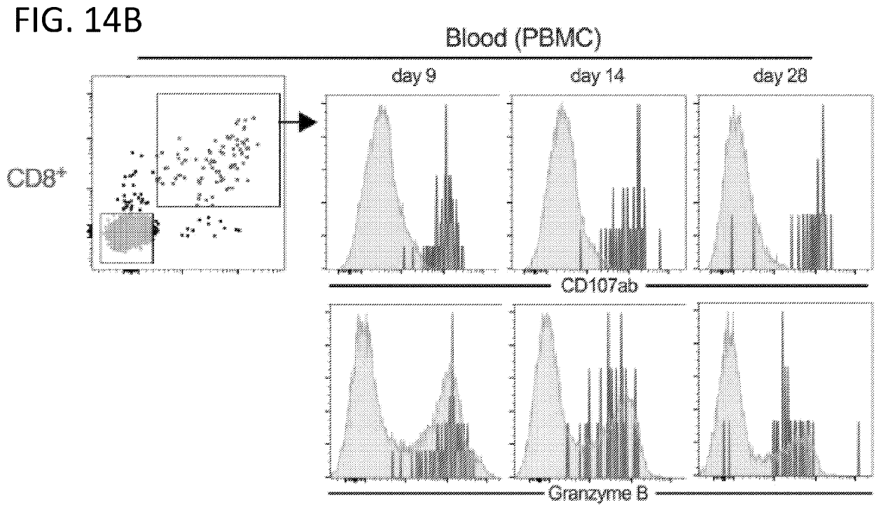

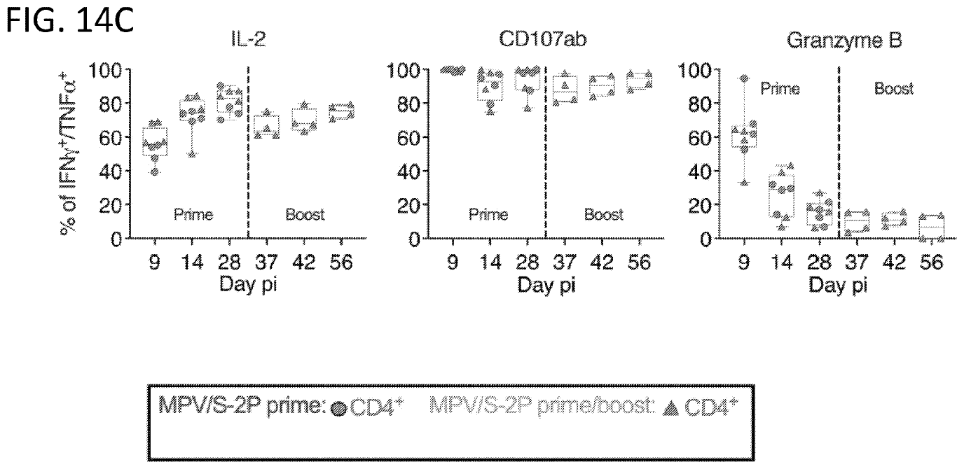

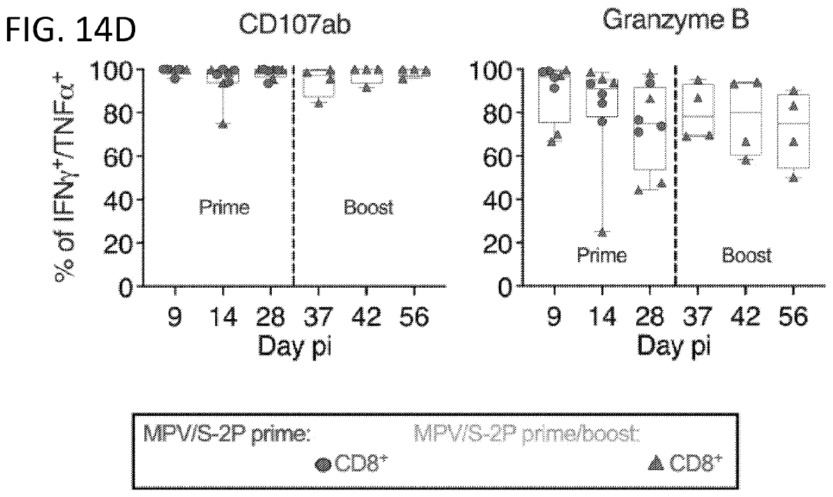

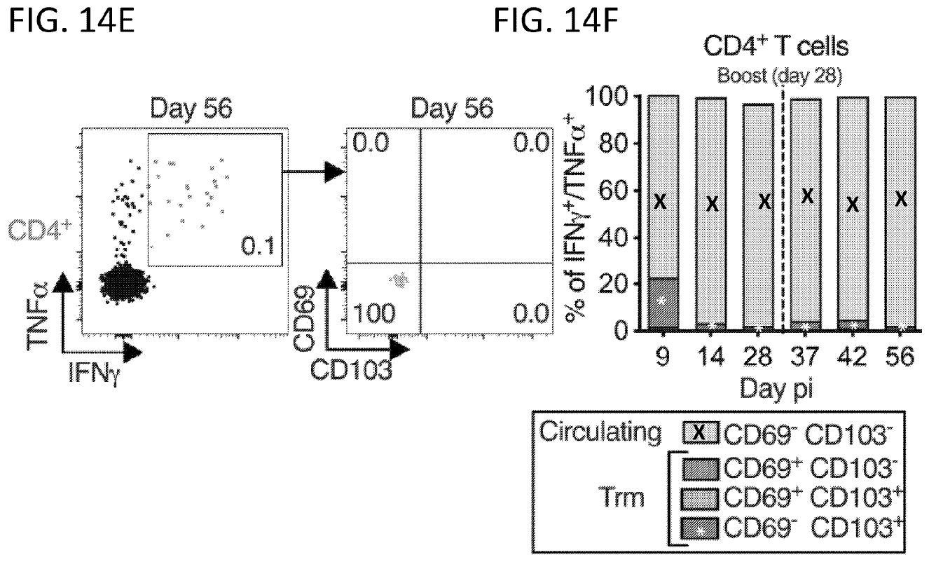

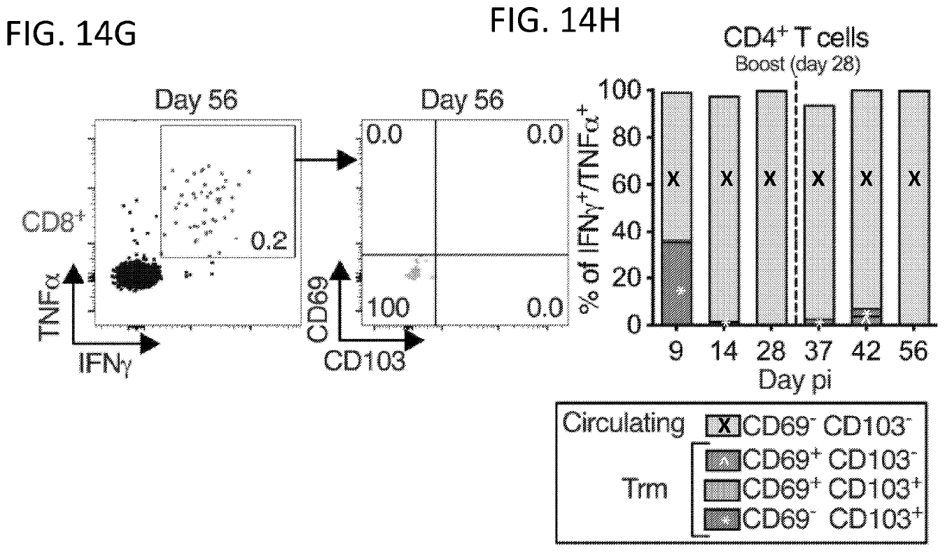

The phenotype of the S-specific T cells in the blood (FIG. 14) and airways, sampled by BAL (FIG. 6), were characterized in greater detail. In addition to expressing IFNγ and TNFα, S-specific CD4⁺ T cells in the airways also expressed IL-2 (FIGS. 6A and 6C), consistent with a Th1-biased phenotype. Subsets of these Th1-biased S-specific CD4⁺ T cells also expressed the degranulation markers CD107ab and granzyme B, suggestive of cytotoxic activity. The large majority of the S-specific IFNγ⁺/TNFα⁺ CD8⁺ T cells in the airways also expressed high levels of CD107ab and granzyme B, suggesting that they exhibited cytotoxic activity and were highly functional (FIGS. 6B and D). The fraction of the S-specific CD4⁺ and CD8⁺ T cells expressing these Th1-related cytokines remained stable after boost (FIGS. 6C and D). Furthermore, the phenotypes of the S-specific T cells in the airways and in the blood were overall comparable (FIGS. 14A-14D). MPV/S-2P induced a small population of S-specific IL-17⁺ CD4⁺ T cells in the LA that peaked on day 14 post-prime (median=0.3%), declined on day 28 and were restimulated after the MPV/S-2P boost (FIGS. 6E and F; median=0.4% on day 42, which was day 14 post-boost).

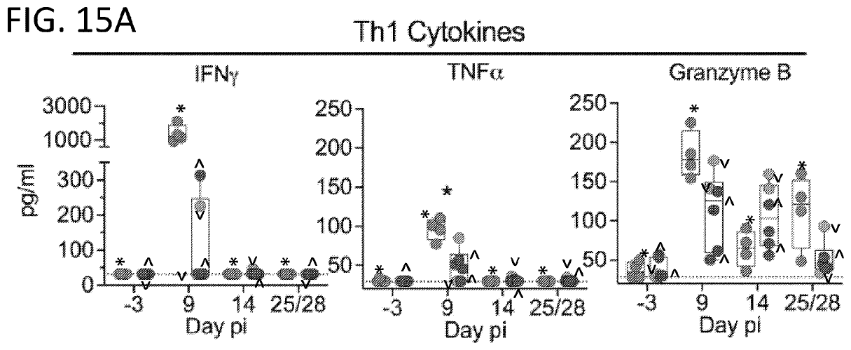

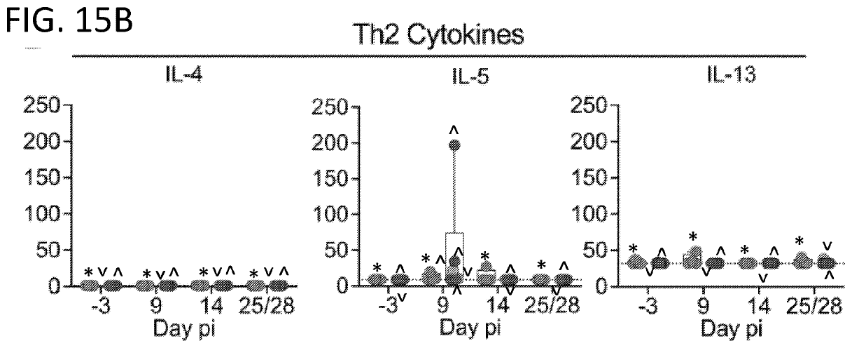

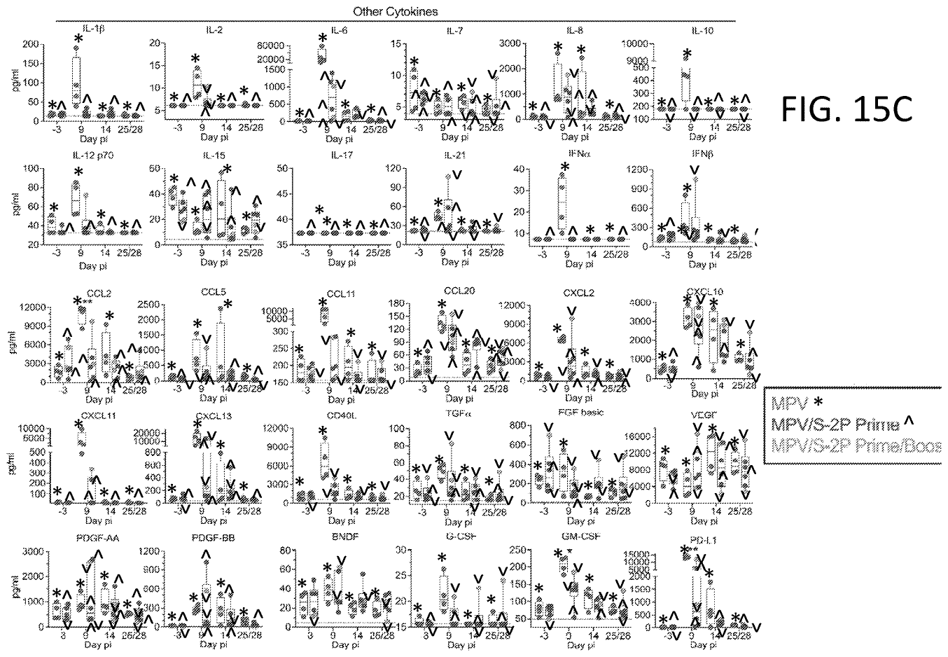

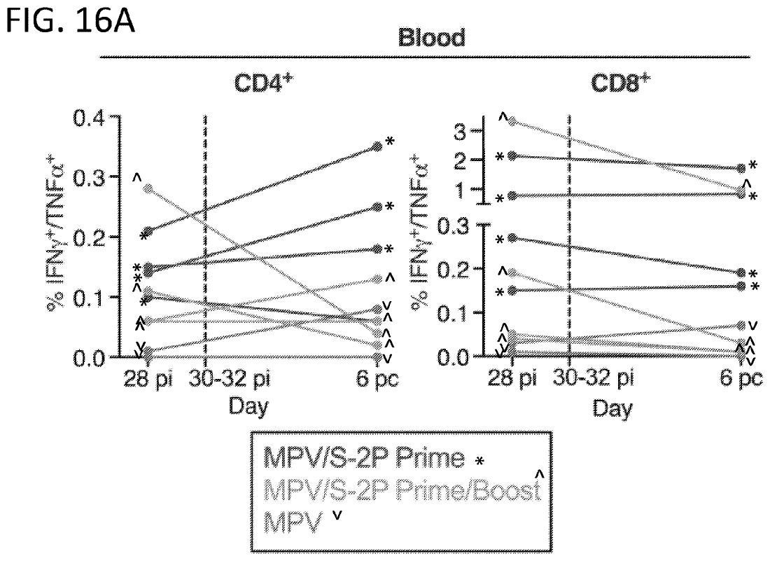

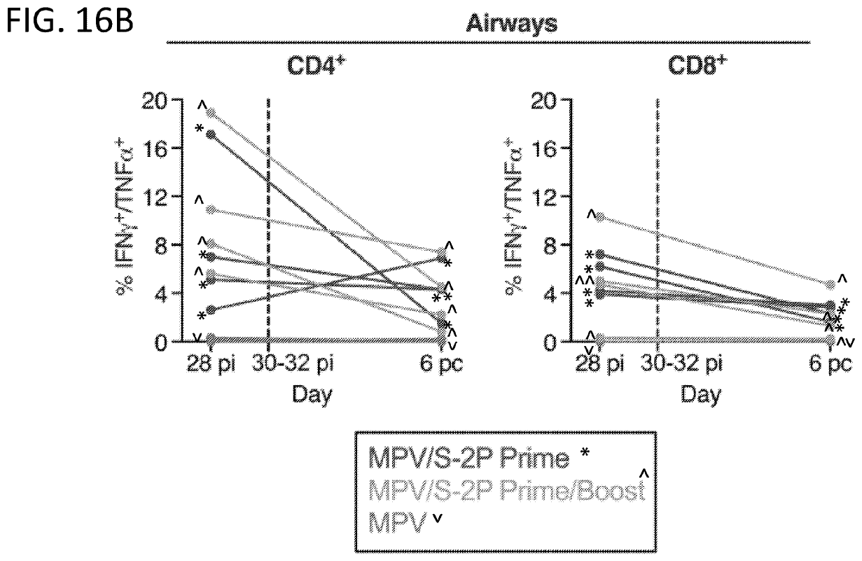

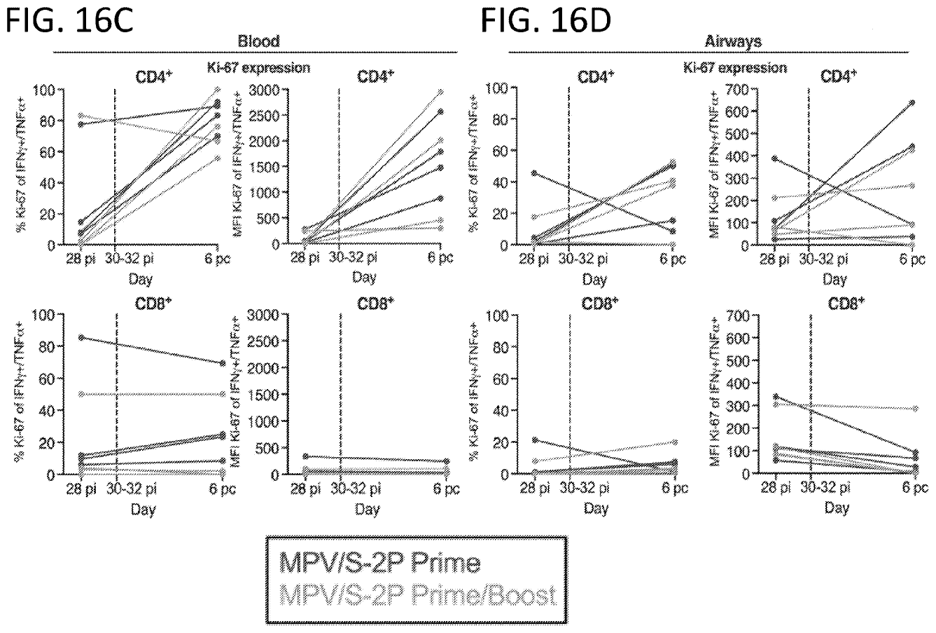

To confirm that immunization with MPV and MPV/S-2P induced a Th1-biased environment in the airways, the expression of 36 cytokines in BAL after the priming was further characterized using a multiplex bead-based immuno-assay (FIG. 15). A transient and moderate increase of the Th1-related cytokines IFNγ, TNFα, and granzyme B was detected on day 9 post-prime with MPV and MPV/S-2P (FIG. 15A). Th2-related cytokines such as IL-4, IL-5, and IL-13 (FIG. 15B) or the Th17-related cytokine IL-17 (FIG. 15C) were not increased after MPV or MPV/S-2P priming. This further confirmed that MPV and MPV/S-2P induced a Th1-biased response in the airways of macaques. Furthermore, a transient and moderate increase of interferons and chemokines also was measured on day 9 post-prime, but notably, the inflammatory response in the airways of MPV/S-2P immunized macaques was significantly milder than in MPV immunized macaques (FIG. 15C).

The phenotype of the S-specific T cells was further characterized by staining for CD69 and CD103 markers of tissue resident memory (Trm) (FIGS. 6G to L). On days 9 and 14 post-prime, the largest fraction of the S-specific IFNγ⁺/TNFα⁻ CD4⁺ and CD8⁺ T cells and IL-17⁺ CD4⁺ T cells in the airways had a circulating phenotype (48 to 71% CD69⁻/CD103⁻ cells, FIGS. 6H, J, and L). However, as early as day 9 post-boost, circulating S-specific T cells transitioned to Trm T cells by the acquisition of CD69 and/or CD103, and by day 56 pi (28 days post-boost), 88% and 82% of S-specific IFNγ⁺/TNFα⁺ CD4⁺ and CD8⁺ T cells (FIGS. 6H and J) and 87% of S-specific IL-17⁺ CD4⁺ T-cells (FIG. 6L) exhibited a Trm phenotype. This transition to a Trm phenotype was specific to T cells from the airways, as S-specific IFNγ⁺/TNFα⁺ CD4⁺ and CD8⁺ T cells in the blood kept a circulating phenotype (FIGS. 14E-14H).

### Example 10: MPV/S-2P Immunized Macaques were Protected from SARS-CoV-2 Challenge Replication in Both the Upper and Lower Airways

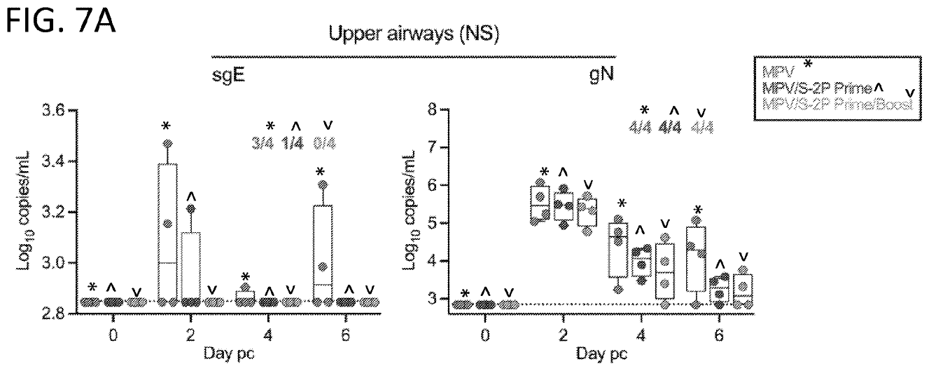

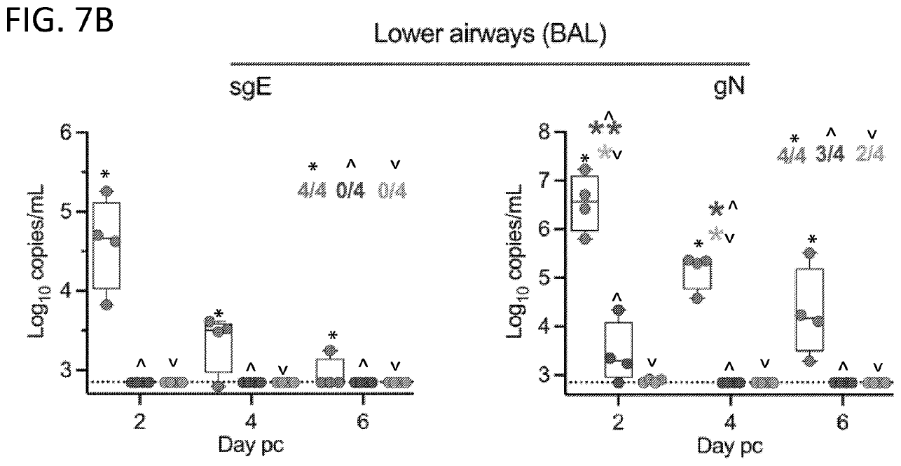

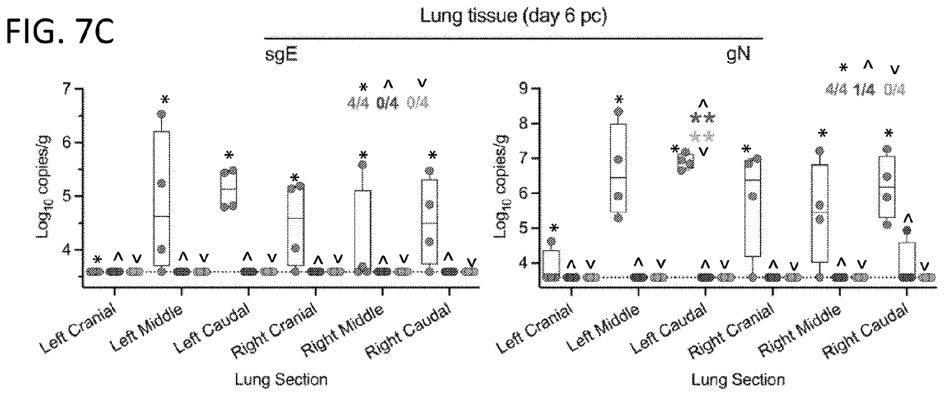

To evaluate the protective efficacy of prime and prime/boost regimens with MPV/S2-P, macaques were challenged by the IN/IT route with 6.3 log₁₀ TCID₅₀ total of the vaccine-matched WA1/2020 strain of SARS-CoV-2. The challenge was performed 31 or 32 days after a single dose of MPV or MPV/S2-P (n=4 per group), or on day 58 (30 days after the boost) for the animals that received two doses of MPV/S-2P (n=4 per group) (FIG. 10). Nasal swabs and BAL were obtained on indicated days post-challenge (pc) to evaluate SARS-CoV-2 replication in the UA and LA. On day 6 pc, animals were euthanized, and lung tissues were obtained. Challenge virus was quantified by RT-qPCR using SARS-CoV-2 specific TaqMan assays for subgenomic E (sgE) or genomic N (gN) mRNA (FIG. 7); sgE mRNA is a marker for active SARS-CoV-2 transcription and replication but is poorly incorporated into mature virus, and gN RNA is a marker for both input and newly-replicated of challenge virus (Dagotto et al., J Virol 95(8):e02370-20, 2021).

By the sgE assay, which distinguishes actively replicating genomes from genomes in mature virions (both input and progeny), NS and BAL of 3/4 and 4/4 macaques immunized with the MPV empty-vector control were positive, and copy numbers were maximal on day 2 pc (medians: 3.0 and 4.7 log₁₀ copies of sgE/ml in the UA and LA, respectively, FIGS. 7A and B, left panels). sgE RNA remained detectable in the UA of 2 of 4 MPV-immunized macaques on day 6 pc. In MPV/S-2P primed macaques, low levels of sgE RNA were detectable only on day 2 pc in the UA of one of 4 animals. No sgE RNA was detected in the UA of any of the macaques that had received a prime/boost immunization with MPV/S-2P, nor in the LA of macaques from the MPV/S-2P prime or prime/boost groups. Thus, SARS-CoV-2 replication was undetectable in the airways of the macaques that had received two IN/IT doses of MPV/S-2P, indicating a high level of protection. SARS-CoV-2 gN RNA was detected in the UA of all macaques on day 2 pi, confirming that all macaques had received challenge virus (FIG. 7A, right panel). In the LA, high copy numbers of gN were mostly measured in MPV-immunized macaques (median of 6.6 log₁₀ copies/ml on day 2 pi. FIG. 7B, right panel), presumably reflecting the presence of mature progeny virions.

After necropsy, high levels of sgE were detected in the lungs of all 4 MPV empty-vector control-immunized macaques (up to 6.5 log₁₀ copies of sgE/g), indicating a high level of active genome replication.

At this day 6 pc time point, all lung tissue specimens from animals that had received one or two doses of MP/2-2P were negative for sgE mRNA, indicative of the absence of replicating challenge virus in animals immunized with one or two IN/IT doses of MPV/S-2P. Similar to sgE mRNA, high copy numbers of gN RNA were measured in most of the lung samples from MPV-immunized macaques (median of 6.4 log₁₀/copies/ml in left middle area of the lungs, FIG. 7C, right panel). Thus, SARS-CoV-2 replication was undetectable in the UA and LA of macaques that received two doses of MPV/S2-P, indicative of a high level of protection.

Finally, the CD4⁺ and CD8⁺ T cell responses in the blood and airways on day 6 post challenge were evaluated, focusing on S-specific IFNγ⁺/TNFα⁺ cells (FIGS. 5B to F). In the blood, a post-challenge increase in S-specific IFNγ⁺/TNFα⁺ CD4⁺ T cells was detected in 3/4 and 1/4 MPV/S-2P-primed and -primed/boosted macaques (FIGS. 5B, C, and 16A), with cells from all animals expressing the proliferation marker Ki-67. In these animals, there was no detectable increase in S-specific blood CD8⁺ T-cells nor an increase in Ki-67⁺ expression in these cells. In the airways, an increase in S-specific IFN≢⁻/TNFα⁻ CD4⁺ and CD8⁺ T cells was not detected after challenge (FIGS. 5E, F and 16B), although the S-specific IFNγ⁺/TNFα⁺ CD4⁺ T cells in the blood of all MPV/S-2P immunized macaques and, in a subset of immunized macaques, in the airways, expressed the proliferation marker Ki-67, suggesting CD4⁺ T cell reactivation by the challenge. There also was no increase in airway S-specific IFNγ⁺/TNFα⁺ CD8⁺ T-cells, and no increase in their Ki-67 expression.

### Example 11: Safety and Immunogenicity of MPV/S-2P Vaccine Candidates

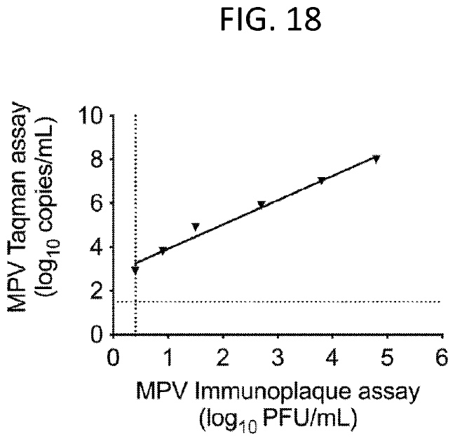

To evaluate safety and immunogenicity of MPV/S-2P vaccine candidates, delivered by nasal sprayer, clinical study material of MPV/S-2P, based on the amino acid sequence of the ancestral virus (GenBank MN908947, Wuhan-Hu-1), and MPV/S-2P-XBB.1.5, encoding an S-2P stabilized version of the S antigen based on the amino acid sequence from the XBB.1.5 variant from a gene added between the NS2 and N genes, were generated under cGMP on Vero cells. The MPV/S-2P final vialed material had a titer of 6.1 log₁₀ PFU per mL. Numerous tests were performed at predetermined time points of the manufacturing process to verify the integrity of the manufacturing process. The final product met selected criteria indicating that it is acceptable for clinical studies and was deemed acceptable for Phase 1 studies. Tenfold serial dilutions of the study material were prepared in Microtest M4RT viral transport medium (Thermo Fisher). Titers of each dilution were confirmed by immunoplaque assay on Vero cells, and copy numbers for each dilution were determined by TAQMAN assay using a primer/probe set specific to the MPV F ORF. Titers determined by MPV immunoplaque assay (log₁₀ PFU per mL) are displayed on the x-axis, and copy numbers (log₁₀ per mL) determined by TAQMAN assay are displayed on the y-axis (FIG. 18). Serial dilutions of a DNA plasmid were used as a standard to determine copy numbers. FIG. 18 shows the linear correlation of immunoplaque titers (log₁₀ PFU/mL) and copy numbers (Log₁₀/mL) (R2=0.9820). The limits of detection of each assay are shown by a dotted line. The results revealed a linear correlation between these two assays.

TABLE 3
Titers of MPV seed stocks for manufacture of clinical study material

| | Titer | |
|---|---|---|
| Virus Name | (Log₁₀ PFU/mL)^{b} | Sequencing result |
| MPV/S-2P | 2.4 × 10⁶ | The consensus sequence perfectly matches that of the |
| | | cDNA from which the recombinant virus was derived. |
| MPV/S-2P-XBB.1.5 | 5.1 × 10⁶ | The consensus sequence perfectly matches that of the |
| | | cDNA from which the recombinant virus was derived. |

### Example 12: Stability of MPV/S-2P at 4-8° C., at Room Temperature, and after Spraying Through a Nasal Atomizer Device

To determine stability and potency of the clinical study material of the MPV/S-2P vaccine candidate, the vaccine potency was determined under conditions simulating use in a clinical study. Results in Table 4 indicate that the potency of the vaccine, diluted in lactated Ringers solution, was stable over 6 hours at 4-8° C. No loss in vaccine titer (potency) was observed after passing of the diluted vaccine through two different models of atomizer sprayer heads, affixed to a 1 mL luer-lock syringe. Results in Table 5 indicate that the vaccine titer (potency) was stable when thawed by rolling of vials between gloved hands or when thawed in a 32° C. water bath. The potency remained stable during subsequent storage at 4-8° C. or at room temperature over 16-17 hours.

TABLE 4
Stability after Thawing on Wet Ice (2-8 °C) of MPV/S-2P,
Diluted in Lactated Ringer's Solution for Injection, and
Stability after Spraying Through Atomizer Device
Assay Result, Stability on Wet Ice and after spraying

| | Assay 1 ^{a} | Assay 2 ^{b} |
|---|---|---|
| | Titer (log₁₀ PFU/mL) | Titer (log₁₀ PFU/mL) |
| Hours | Diluent: LR | Diluent: LR |
| 0 | 5.6 | 5.9 |
| 0 (VAX300) | 5.6 | Nd |
| 0 (MAD300) | Nd | 5.8 |
| 2 | 5.6 | 5.7 |
| 4 | 5.7 | 5.9 |
| 6 | 5.8 | 5.8 |
| Range: | 5.6-5.8 | 5.7-5.9 |

LR = Lactated Ringer's Solution for Injection, USP. Nd, not done.
^{a} A vial was thawed in a 37 °C water bath and diluted with Lactated Ringer's Solution for Injection. The diluted vaccine was filled into a 1 mL luer-lock syringe (Beckton Dickinson; BD). A VAX300 sprayer head (Teleflex) was affixed to the luer-lock syringe. The diluted vaccine was sprayed through the VAX300 sprayer head, and the spray was captured in a 1.6 mL conical tube prior to titration. A second aliquot of the diluted vaccine was stored on wet ice, protected from light, and titrations were performed on Vero cells at indicated time points.
^{b} A vial was thawed in a 37 °C water bath and diluted with Lactated Ringer's Solution for Injection. A MAD300 sprayer head (Teleflex) was affixed to a 1 mL luer-lock syringe (BD). The diluted vaccine was sprayed through the MAD300 sprayer head (Teleflex), and the spray was captured in a 1.6 mL conical tube prior to titration. A second aliquot of the diluted vaccine was stored on wet ice, protected from light, and titrations were performed on Vero cells at indicated time points.

TABLE 5
Thawing Conditions of MPV/S-2P and stability at room temperature and 4-8 °C.
Assay Results, Thawing Conditions and Storage

| Vial | | Titer | Stability after 16-17 Hours^{d} | |
|---|---|---|---|---|
| Number | Thawing Condition | (log₁₀ PFU/mL) | Storage Condition | Titer (log₁₀ PFU/mL) |
| 1 | Rolling between | 6.0 | Refrigerator; | 5.9 |
| 2 | Gloved Hands ^{a} | 6.2 | 4-8 °C. | 5.9 |
| 3 | | 6.0 | | 5.9 |
| Range: | | 6.0-6.2 | 5.9 | |
| 4 | 32 °C; | 5.9 | Tube Rack; | 5.7 |
| 5 | Water Bath ^{b} | 5.9 | Room | 5.8 |
| 6 | | 5.9 | Temperature | 5.9 |
| Range: | | 5.9 | 5.7-5.9 | |
| 7 | Tube Rack; | 5.7 | Tube Rack; | 5.6 |
| | Room | | Room | |
| | Temperature ^{c} | | Temperature | |

^{a} Vials were thawed by rolling between gloved hands for 4-5 minutes. A volume of about 50 μl was removed from each vial for titration on Vero cells.
^{b} Vials were thawed in a 32 °C water bath for 3 minutes. A volume of about 50 μl was removed from each vial for titration on Vero cells.
^{c} One vial was thawed by incubation in a tube rack on the bench top at room temperature for 20 minutes, protected from light. A volume of about 20 μl was removed from the vial for titration on Vero cells.
^{d}The remaining volume in each vial was stored as indicated for an additional period of 16-17 hours, protected from light. Titrations were subsequently performed on Vero cells.

It will be apparent that the precise details of the methods or compositions described may be varied or modified without departing from the spirit of the described aspects of the disclosure. We claim all such modifications and variations that fall within the scope and spirit of the claims below.

1. A live-attenuated recombinant murine pneumonia virus (rMPV), comprising:
a genome comprising in a 3′ to 5′ order, a 3′ leader sequence, an MPV non-structural protein 1 (NS1) gene, an MPV non-structural protein 2 (NS2) gene, a heterologous gene, an MPV nucleoprotein (N) gene, an MPV phosphoprotein (P) gene, an MPV matrix protein (M) gene, an MPV small hydrophobic protein (SH) gene, an MPV attachment glycoprotein (G) gene, an MPV fusion glycoprotein (F) gene, an MPV M2 gene, an MPV polymerase protein (L) gene, and a 5′ trailer sequence,
wherein the heterologous gene encodes a severe acute respiratory syndrome coronavirus 2 (SARS-CoV-2) spike (S) protein comprising R682G, R683S, R685S, K986P, and V987P amino acid substitutions, wherein the amino acid positions are according to the reference SARS-CoV-2 S protein sequence set forth as SEQ ID NO: 33.

2. The rMPV of claim 1, wherein the S protein further comprises:
F817P, A892P, A899P, and A942P amino acid substitutions; or
Q52H and F456L amino acid substitutions.

3. The rMPV of claim 1, wherein the heterologous gene is flanked by MPV gene start (GS) and gene end (GE) transcription signals.

4. The rMPV of claim 1, wherein the S protein open reading frame (ORF) is transcribed by the MPV polymerase complex as a distinct mRNA.

5. The rMPV of claim 1, wherein the heterologous gene is codon-optimized for expression in human cells.

6. The rMPV of claim 1, wherein:
a Kozak consensus sequence (GCCGCCACC) or a modified Kozak consensus sequence with reduced G/C content is inserted immediately upstream of the heterologous gene start codon; or
an adenosine residue is inserted three nucleotides upstream (−3) of the heterologous gene start codon.

7. (canceled)

8. The rMPV of claim 1, wherein the L gene is codon pair optimized for expression in human cells.

9. The rMPV of claim 1, wherein the amino acid sequence of the S protein is at least 90% identical to SEQ ID NO: 33, SEQ ID NO: 34, SEQ ID NO: 36, SEQ ID NO: 37, SEQ ID NO: 38, SEQ ID NO: 39, SEQ ID NO: 54 or SEQ ID NO: 55.

10. (canceled)

11. The rMPV of claim 1, wherein the amino acid sequence of the S protein comprises or consists of SEQ ID NO: 33, SEQ ID NO: 34, SEQ ID NO: 36, SEQ ID NO: 37, SEQ ID NO: 38, SEQ ID NO: 39, SEQ ID NO: 54 or SEQ ID NO: 55.

12. The rMPV of claim 1, wherein the genome comprises an antigenomic cDNA sequence at least 95% identical to SEQ ID NO: 9, SEQ ID NO: 11, SEQ ID NO: 25, SEQ ID NO: 27, SEQ ID NO: 29, SEQ ID NO: 31, SEQ ID NO: 50, or SEQ ID NO: 52.

13. The rMPV of claim 1, wherein the genome comprises an antigenomic cDNA sequence comprising or consisting of SEQ ID NO: 9, SEQ ID NO: 11, SEQ ID NO: 25, SEQ ID NO: 27, SEQ ID NO: 29, SEQ ID NO: 31, SEQ ID NO: 50, or SEQ ID NO: 52.

14. A nucleic acid molecule comprising the nucleotide sequence of the genome of the rMPV of claim 1, or an antigenomic cDNA or RNA sequence of the genome.

15. A vector comprising the nucleic acid molecule of claim 14.

16. The vector of claim 15, further comprising a promoter operably linked to the nucleic acid molecule.

17. The vector of claim 16, wherein the promoter is a T7 promoter.

18. The vector of claim 15, further comprising a ribozyme sequence and a transcription terminator operably linked to the nucleic acid molecule.

19. The vector of claim 18, wherein the last nucleotide of the 5′ trailer sequence is followed by three guanosine residues, a hepatitis delta virus ribozyme sequence, and a T7 terminator.

20. The vector of claim 15, wherein the nucleotide sequence of the vector is at least 95% identical to SEQ ID NO: 10, SEQ ID NO: 12, SEQ ID NO: 26, SEQ ID NO: 28, SEQ ID NO: 30, SEQ ID NO: 32, SEQ ID NO: 51 or SEQ ID NO: 53.

21. The vector of claim 15, wherein the nucleotide sequence of the vector comprises or consists of SEQ ID NO: 10, SEQ ID NO: 12, SEQ ID NO: 26, SEQ ID NO: 28, SEQ ID NO: 30, SEQ ID NO: 32, SEQ ID NO: 51 or SEQ ID NO: 53.

22. An isolated host cell comprising the nucleic acid molecule of claim 14.

23. An in vitro method of producing a recombinant MPV (rMPV), comprising:
transfecting MPV-permissive cells with the vector of claim 15, and plasmids encoding MPV N, P, L and M2-1 proteins;
incubating the cell culture for a period of time sufficient to allow for viral replication; and
purifying the replicated virus to produce the rMPV.

24. The method of claim 23, wherein the MPV-permissive cells express T7 RNA polymerase, or the MPV-permissive cells are transfected with a plasmid encoding T7 RNA polymerase.

25. A rMPV produced by the method of claim 23.

26. An immunogenic composition comprising a pharmaceutically acceptable carrier and the rMPV of claim 1.

27. The immunogenic composition of claim 26, further comprising an adjuvant and/or a secondary therapeutic agent.

28. (canceled)

29. The immunogenic composition of claim 27, wherein the secondary therapeutic agent comprises a decongestant, an antihistamine, a pain reliever, a fever reducer, a cough suppressant, a cytokine, an antibiotic, an anti-viral agent, or any combination thereof.

30. The immunogenic composition of claim 26, which is formulated for intranasal administration.

31. The immunogenic composition of claim 30, which is formulated as a spray, mist, or aerosol.

32. A method of eliciting an immune response against SARS-CoV-2 in a subject, comprising administering to the subject an effective amount of the immunogenic composition of claim 26.

33. The method of claim 32, wherein the immunogenic composition is administered intranasally.

34. The method of claim 33, wherein the immunogenic composition is administered as a spray, mist, or aerosol.

35. The method of claim 32, wherein the immunogenic composition is administered in a single dose or in two doses.

36. (canceled)

37. The method of claim 32, wherein the subject is a human subject.

38. The method of claim 32, wherein the immune response is a protective immune response.

39. The method of claim 32, wherein; the immunogenic composition is administered as a prime dose in a prime-boost dosing regimen; or
the immunogenic composition is administered as a boost dose in a prime-boost dosing regimen.

40-41. (canceled)
